(19) United States
(12) Patent Application Publication
(10) Pub. No.: US 2006/0293309 A1
(43) Pub. Date: Dec. 28, 2006

(54) Method of treating disorders and conditions using peripherally-restricted antagonists and inhibitors

(75) Inventors: Karl Bruce Thor, Cary, NC (US); Daniel J. Ricca, Rougemont, NC (US)

Correspondence Address: LAHIVE & COCKFIELD, LLP, ONE POST OFFICE SQUARE, BOSTON, MA, 02109-2127, US

(73) Assignee: Dynogen Pharmaceuticals, Inc., Waltham, MA (US)

(21) Appl. No.: 11/389,887

(22) Filed: Mar. 27, 2006

Related U.S. Application Data
(60) Provisional application No. 60/666,253, filed on Mar. 28, 2005.

(51) Int. Cl. A61K 31/551; A61K 31/519

(52) U.S. Cl. 514/218; 514/252.16

(57) ABSTRACT

The instant invention features compounds, for example, 5-HT₃ receptor antagonists, having a peripherally restricted mode of action such that the compounds affect 5-HT₃ receptors of the peripheral nervous system with diminished or reduced effects in the central nervous system. The compounds are particularly useful in treating disorders or conditions ameliorated by antagonism of peripheral 5-HT₃ receptors. Moreover, side-effects attributable to antagonism of central 5-HT₃ receptors can be lessened or reduced using the peripherally restricted compounds of the invention.

### RELATED APPLICATIONS

This application claims the benefit of priority under 35 U.S.C. §119(e) to U.S. Provisional Application Ser. No. 60/666,253, filed Mar. 28, 2005, the content of which is hereby incorporated herein by this reference in its entirety.

### BACKGROUND OF THE INVENTION

Monoamine neurotransmitter receptor antagonists, such as antagonists of serotonin are currently used therapeutically. The general class of serotonin receptors is referred to as the 5-HT (5-hydroxytryptamine) receptors. Specific 5-HT receptors include the 5-HT_{1A}, 5-HT_{1B}, 5-HT_{1D}, 5-HT_{1E}, 5-HT_{1F}, 5-HT_{1P}, 5-HT_{1S}, 5-HT_{2A}, 5-HT_{2B}, 5-HT_{2C}, 5-HT₃, 5-HT₄, 5-HT₅, 5-HT₆, and 5-HT₇ receptors. Each of these receptors mediates certain physiological effects. See Leonard, B. E., International Clinical Psychopharmacology, 7:13-21 (1992). 5HT₃ receptors are found both in the Central Nervous System (CNS) and in the Peripheral Nervous System (PNS).

One important class of 5HT receptor antagonists are the 5HT₃ antagonists which are used to treat numerous disorders and/or conditions such as post-operative nausea and vomiting, chemotherapy induced nausea and vomiting, radiotherapy induced nausea and vomiting, anxiety, psychosis, drug and alcohol abuse, eating disorders, depression, cognition, pain and irritable bowl syndrome.

However, current 5HT₃ receptor antagonists can produce side-effects that reduce desirability of these agents. For example, hypertension, headache, dizziness, and gastrointestinal disturbances (e.g., diarrhea or constipation) have all been reported. In fact, severe complications of constipation that resulted in 44 hospitalizations and 5 deaths prompted the manufacturer of one 5HT₃ receptor antagonist to withdraw the drug from the market. These complications included intestinal blockages, extreme inflammation and distention of the large intestine, and compromised blood flow to the colon (ischemic colitis).

Therefore, there is a need for improved 5HT₃ receptor antagonists with reduced side effects, in particular, side effects mediated by interaction of 5HT₃ receptor antagonists with serotonin receptors in the CNS, for use in therapy. The development of such antagonists would be of great benefit in the treatment of numerous 5HT₃-mediated conditions.

### SUMMARY OF THE INVENTION

A need exists for the development of new compositions and methods useful for the improved treatment of 5-HT₃ mediated disorders and/or conditions. The instant invention features such compositions and methods. In particular, the invention features compounds, for example, 5-HT₃ receptor antagonists, having a peripherally restricted mode of action such that the compounds affect 5-HT₃ receptors of the peripheral nervous system with diminished or reduced effects in the central nervous system. The compounds are particularly useful in treating disorders or conditions ameliorated by antagonism of peripheral 5-HT₃ receptors. Moreover, side-effects attributable to antagonism of central 5-HT₃ receptors can be lessened or reduced using the peripherally restricted compounds of the invention. In exemplary embodiments, the methods of the invention comprise administering to a subject in need of treatment a therapeutically effective amount of a compound that has peripherally-restricted 5-HT₃ receptor antagonist activity. Such compounds possess enhanced therapeutic profiles as compared with existing compounds used for treatment of 5-HT₃ mediated disorders.

In certain aspects, the disease or disorder (or at lease one symptom of the disease or disorder) is directly mediated by 5-HT₃ activity. In such aspects, compounds of the invention directly treat the disease or disorder, for example, improving (e.g., alleviating, easing or lessening) one or more symptoms of a disease or disorder, such symptoms resulting from 5-HT₃ receptor activity of the peripheral nervous system. In other aspects, the disease or disorder (or at lease one symptom of the disease or disorder) is indirectly mediated by 5-HT₃ receptor activity. In such aspects, the compounds of the invention have an indirect effect, for example, improving one or more symptoms indirectly associated with a 5-HT₃ mediated disease or disorder.

In certain aspects, a significant component (or components) of the disease or disorder is mediated by peripheral 5-HT₃ receptor activity. In other aspects, a significant component (or components) of the disease or disorder is mediated by 5-HT₃ receptor activity in the gastrointestinal (GI) tract. The compounds of the invention are particularly useful for the treatment of such peripheral and/or GI components of the disease or disorder due, at least in part, to the reduced membrane permeability, peripherally restricted activity and/or low bioavailability of the compounds. In other aspects, the compounds can be used to treat central nervous system (CNS) diseases or disorders (or CNS-mediated components of a disease or disorder), for example, by direct administration of the compounds to the CNS.

In certain aspects of the invention, the bioavailability of the 5-HT₃ receptor antagonists of the invention is lower than the parent, e.g., non-quaternary 5-HT₃ receptor antagonists, from which they were derived. In one aspect, the 5-HT₃ receptor antagonists have retarded bioavailability as compared to the parent, non-quaternary 5-HT₃ receptor antagonists from which they were derived. Without wishing to be bound in theory, this retarded or lower bioavailability is believed to be due to the compounds being more water-soluble as compared to the parent compounds and, accordingly, less likely to cross membranes, e.g., gut membranes. This lower bioavailability is presumed to be advantageous in the treatment of several 5-HT₃ mediated disorders, for example, in certain gastrointestinal disorders.

Another aspect of the invention relates to a method for treating one or more 5-HT₃ mediated disorders in a subject in need thereof comprising administering to said subject a therapeutically effective amount of a compound of Formula I: 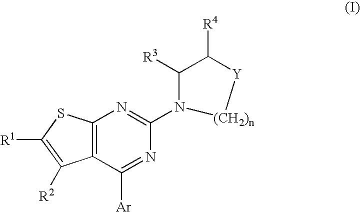
wherein R₁ and R₂ independently represent hydrogen, halogen or a C₁-C₆ alkyl group;
or R₁ and R₂ together with the carbon atom to which they are attached form a cycloalkyl group having 5 to 6 carbon atoms;
R₃ and R₄ independently represent hydrogen or a C₁-C₆ alkyl group;
Y represents a peripherally-restricted moiety;
Ar is a substituted or unsubstituted phenyl, 2-thienyl or 3-thienyl group; and n is 2 or 3;
or a pharmaceutically acceptable salt thereof.

In an additional aspect, the invention is directed to a method for treating one or more 5-HT₃ mediated disorders thereof in a subject in need thereof comprising administering to said subject a therapeutically effective amount of a compound of Formula II: 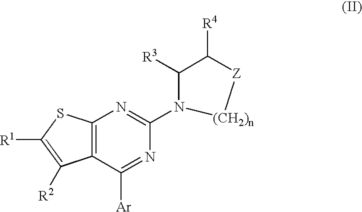
wherein R₁ and R₂ independently represent hydrogen, halogen or a C₁-C₆ alkyl group;
or R₁ and R₂ together with the carbon atom to which they are attached form a cycloalkyl group having 5 to 6 carbon atoms;
R₃ and R₄ independently represent hydrogen or a C₁-C₆ alkyl group;
Z represents a quaternary ammonium moiety;
Ar is a substituted or unsubstituted phenyl, 2-thienyl or 3-thienyl group;
and n is 2 or 3;
or a pharmaceutically acceptable salt thereof.

In another aspect, the invention is directed to a method for treating one or more 5-HT₃ mediated disorders in a subject in need thereof comprising administering to said subject a therapeutically effective amount of a compound of Formula III: 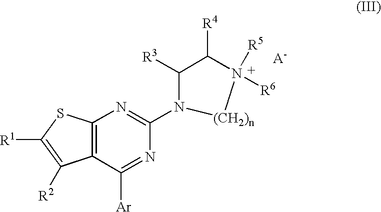
wherein R₁ and R₂ independently represent hydrogen, halogen or a C₁-C₆ alkyl group;
or R₁ and R₂ together with the carbon atom to which they are attached form a cycloalkyl group, e.g., C₃-C₈;
R₃ and R₄ independently represent hydrogen, a C₁-C₆ alkyl group;
R₅ and R₆ independently represent C₁-C₆ alkyl, 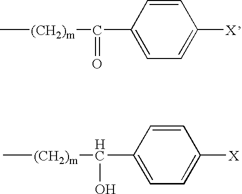
—C(O)—NH—R₇ wherein m is an integer from about 1 to about 3, X is halogen and R₇ is a C₁-C₆ alkyl group, R₅ and R₆ taken together form a cycloalkyl group, e.g., C₃-C₈, or one of R₅ and R₆ is O⁻;
Ar is a substituted or unsubstituted phenyl, 2-thienyl or 3-thienyl group;
A⁻ represents a pharmaceutically acceptable anion;
and n is 2 or 3;
or a pharmaceutically acceptable salt thereof.

In another aspect, the invention is directed to a method for treating, preventing or reducing one or more 5-HT₃ mediated disorders in a subject in need thereof comprising administering to said subject a therapeutically effective amount of a quaternary ammonium derivative of MCI-225 (MCI-225-QUAT). Another aspect of the invention is directed to a method for treating, preventing or reducing one or more 5-HT₃ mediated disorders in a subject in need thereof comprising administering to said subject a therapeutically effective amount of a quaternary ammonium derivative of a thieno[2,3-d]pyrimidine. MCI-225 and other thieno[2,3-d]pyrimidine derivatives are described, for example, in U.S. Pat. No. 4,695,568 and U.S. patent application Ser. Nos. 10/757,364, 10/757,981, 10/817,332 and 10/617,847, the entire contents of which are incorporated herein by reference.

In one embodiment, the 5-HT₃ mediated disorder is a functional bowel disorder, e.g., irritable bowel syndrome (IBS). In an exemplary embodiment, the 5-HT₃ mediated disorder is diarrhea-predominant irritable bowel syndrome (IBS-d). In another embodiment, the 5-HT₃ mediated disorder is a lower urinary tract disorder, e.g., overactive bladder (OAB) including urge incontinence, or stress urinary incontinence. In another embodiment, the 5-HT₃ mediated disorder is nausea, vomiting/emesis, or retching. In an exemplary embodiment, the 5-HT₃ mediated disorder is chronic functional vomiting (CFV). In another embodiment, the 5-HT₃ mediated disorder is pain. In another embodiment, the 5-HT₃ mediated disorder is a depressive condition. In another embodiment, the 5-HT₃ mediated disorder is selected from the group consisting of obesity and weight gain, eating disorders, pre-menstrual syndrome, fibromyalgia, migraine, Parkinson's disease, stroke, schizophrenia, obsessive-compulsive disorder, fatigue, and any combination thereof.

In a particular embodiment, the 5-HT₃ mediated disorder is selected from the group consisting of any one of the above-noted 5-HT₃ mediated disorders, or a combination thereof.

In another aspect, the invention is directed to a method for treating one or more 5-HT₃ mediated disorders in a subject in need thereof comprising coadministering to said subject a peripherally-restricted 5-HT₃ receptor antagonist with an additional agent.

In another aspect, the invention is directed to a packaged pharmaceutical composition for treating one or more 5-HT₃ mediated disorders in a subject, comprising a container holding a therapeutically effective amount of a peripherally-restricted 5-HT₃ receptor antagonist; and instructions for using the antagonist for treating one or more 5-HT₃ mediated disorders in a subject.

Another aspect of the invention pertains to a packaged pharmaceutical composition for treating one or more 5-HT₃ mediated disorders in a subject, comprising a container holding a therapeutically effective amount of a peripherally-restricted 5-HT₃ receptor antagonist; and instructions for using the antagonist and an additional agent for treating one or more 5-HT₃ mediated disorders thereof in a subject.

In an additional aspect, the invention relates to a pharmaceutical composition comprising a peripherally-restricted 5-HT₃ receptor antagonist and a pharmaceutically acceptable carrier for treating one or more 5-HT₃ mediated disorders in a subject, wherein the peripherally-restricted 5-HT₃ receptor antagonist is selected based on its peripheral restriction, e.g., an MCI-225-QUAT.

Another aspect of the invention is a pharmaceutical composition comprising a peripherally-restricted 5-HT₃ receptor antagonist, an additional agent and a pharmaceutically acceptable carrier for treating one or more 5-HT₃ mediated disorders in a subject. An additional aspect of the invention pertains to a compound of Formula I, Formula II or Formula III, described herein.

In one aspect, the invention relates to a method for treating a functional bowel disorder, e.g., at least one symptom of a functional bowel disorder, in a subject in need thereof comprising administering to said subject a therapeutically effective amount of a quaternary ammonium derivative of MCI-225 (MCI-225-QUAT).

Another aspect of the invention relates to a method for treating a functional bowel disorder, e.g., at least one symptom of a functional bowel disorder, in a subject in need thereof comprising administering to said subject a therapeutically effective amount of a compound of Formula I, Formula II or Formula III, described herein.

In another aspect, the invention is directed to a method for treating a functional bowel disorder, e.g., at least one symptom of a functional bowel disorder, in a subject in need thereof comprising coadministering to said subject a peripherally-restricted 5-HT₃ receptor antagonist with an additional agent.

In another aspect, the invention is directed to a packaged pharmaceutical composition for treating a functional bowel disorder, e.g., at least one symptom of a functional bowel disorder, in a subject, comprising a container holding a therapeutically effective amount of a peripherally-restricted 5-HT₃ receptor antagonist; and instructions for using the antagonist for treating a functional bowel disorder in a subject.

Another aspect of the invention pertains to a packaged pharmaceutical composition for treating a functional bowel disorder, e.g., at least one symptom of a functional bowel disorder, in a subject, comprising a container holding a therapeutically effective amount of a peripherally-restricted 5-HT₃ receptor antagonist; and instructions for using the antagonist and an additional agent for treating a functional bowel disorder in a subject.

In another aspect, the invention is directed to a pharmaceutical composition comprising a peripherally-restricted 5-HT₃ receptor antagonist and a pharmaceutically acceptable carrier for treating a functional bowel disorder, e.g., at least one symptom of a functional bowel disorder, in a subject, wherein the peripherally-restricted 5-HT₃ receptor antagonist is selected based on its peripheral restriction, e.g., an MCI-225-QUAT.

In yet another aspect, the invention is directed to a pharmaceutical composition comprising a peripherally-restricted 5-HT₃ receptor antagonist, an additional agent and a pharmaceutically acceptable carrier for treating a functional bowel disorder in a subject.

Another aspect of the invention is a method of treating a lower urinary tract disorder, e.g., a symptom of a lower urinary tract disorder, in a subject in need thereof comprising administering to said subject a therapeutically effective amount of a quaternary ammonium derivative of MCI-225 (MCI-225-QUAT).

Another aspect of the invention relates to a method of treating a lower urinary tract disorder, e.g., a symptom of a lower urinary tract disorder, in a subject in need thereof comprising administering to said subject a therapeutically effective amount of a compound of Formula I, Formula II or Formula III, described herein.

In yet another aspect, the invention pertains to a method of treating, e.g., at least one symptom of a lower urinary tract disorder, in a subject in need of treatment, e.g., wherein the symptom is selected from the group consisting of urinary frequency, urinary urgency, nocturia and enuresis, comprising coadministering to said subject a peripherally-restricted 5-HT₃ receptor antagonist with an additional agent.

In another aspect, the invention is directed to a packaged pharmaceutical composition for treating a lower urinary tract disorder, e.g., a symptom of a lower urinary tract disorder, in a subject, comprising a container holding a therapeutically effective amount of a peripherally-restricted 5-HT₃ receptor antagonist; and instructions for using the antagonist for treating at least one symptom of a lower urinary tract disorder in a subject.

Another aspect of the invention pertains to a packaged pharmaceutical composition for treating a lower urinary tract disorder, e.g., a symptom of a lower urinary tract disorder, in a subject, comprising a container holding a therapeutically effective amount of a peripherally-restricted 5-HT₃ receptor antagonist; and instructions for using the antagonist and an additional agent for treating at least one symptom of a lower urinary tract disorder in a subject.

Another aspect of the invention is a pharmaceutical composition comprising a peripherally-restricted 5-HT₃ receptor antagonist and a pharmaceutically acceptable carrier for treating at least one symptom of a lower urinary tract disorder in a subject in need of treatment, wherein the symptom is selected from the group consisting of urinary frequency, urinary urgency, nocturia and enuresis, wherein the peripherally-restricted 5-HT₃ receptor antagonist is selected based on its peripheral restriction, e.g., an MCI-225-QUAT.

In an additional aspect, the invention relates to a pharmaceutical composition comprising a peripherally-restricted 5-HT₃ receptor antagonist, an additional agent and a pharmaceutically acceptable carrier for treating a lower urinary tract disorder, e.g., a symptom of a lower urinary tract disorder, in a subject in need of treatment, wherein the symptom is selected from the group consisting of urinary frequency, urinary urgency, nocturia and enuresis.

Another aspect of the invention is a method for treating urinary incontinence in a subject in need thereof comprising administering to said subject a therapeutically effective amount of a quaternary ammonium derivative of MCI-225 (MCI-225-QUAT).

An additional aspect of the invention is directed to a method for treating urinary incontinence in a subject in need thereof comprising coadministering to said subject a peripherally-restricted 5-HT₃ receptor antagonist with an additional agent.

In another aspect, the invention is directed to a packaged pharmaceutical composition for treating urinary incontinence in a subject, comprising a container holding a therapeutically effective amount of a peripherally-restricted 5-HT₃ receptor antagonist; and instructions for using the antagonist for treating urinary incontinence in a subject.

Another aspect of the invention pertains to a packaged pharmaceutical composition for treating urinary incontinence in a subject, comprising a container holding a therapeutically effective amount of a peripherally-restricted 5-HT₃ receptor antagonist; and instructions for using the antagonist and an additional agent for treating urinary incontinence in a subject.

In another aspect, the invention relates to a pharmaceutical composition comprising a peripherally-restricted 5-HT₃ receptor antagonist and a pharmaceutically acceptable carrier for treating urinary incontinence in a subject in need thereof, wherein the peripherally-restricted 5-HT₃ receptor antagonist is selected based on its peripheral restriction.

Another aspect of the invention is a pharmaceutical composition comprising a peripherally-restricted 5-HT₃ receptor antagonist, an additional agent and a pharmaceutically acceptable carrier for treating urinary incontinence in a subject.

Another aspect of the invention is a method for treating nausea, vomiting, retching or any combination thereof in a subject in need thereof comprising administering to said subject a therapeutically effective amount of a quaternary ammonium derivative of MCI-225 (MCI-225-QUAT).

Another aspect of the invention relates to a method for treating nausea, vomiting, retching or any combination thereof in a subject in need thereof comprising administering to said subject a therapeutically effective amount of a compound of Formula I, Formula II or Formula III, described herein.

In yet another aspect, the invention pertains to a method for treating nausea, vomiting, retching or any combination thereof in a subject in need thereof comprising coadministering to said subject a peripherally-restricted 5-HT₃ receptor antagonist with an additional agent.

In another aspect, the invention is directed to a packaged pharmaceutical composition for treating nausea, vomiting, retching or any combination thereof in a subject, comprising a container holding a therapeutically effective amount of a peripherally-restricted 5-HT₃ receptor antagonist; and instructions for using the antagonist for treating nausea, vomiting, retching or any combination thereof in a subject.

Another aspect of the invention pertains to a packaged pharmaceutical composition for treating nausea, vomiting, retching or any combination thereof in a subject, comprising a container holding a therapeutically effective amount of a peripherally-restricted 5-HT₃ receptor antagonist; and instructions for using the antagonist and an additional agent for treating nausea, vomiting, retching or any combination thereof in a subject.

In an additional aspect, the invention relates to a pharmaceutical composition comprising a peripherally-restricted 5-HT₃ receptor antagonist and a pharmaceutically acceptable carrier for treating nausea, vomiting, retching or any combination thereof in a subject, wherein the peripherally-restricted 5-HT₃ receptor antagonist is selected based on its peripheral restriction, e.g., an MCI-225-QUAT.

Another aspect of the invention is a pharmaceutical composition comprising a peripherally-restricted 5-HT₃ receptor antagonist, an additional agent and a pharmaceutically acceptable carrier for treating nausea, vomiting, retching or any combination thereof in a subject.

### DETAILED DESCRIPTION OF THE INVENTION

The invention relates to methods of treating vomiting, nausea, retching, lower urinary tract disorders, functional bowel disorders, and other 5-HT₃ mediated disorders in a subject in need of treatment. The methods comprise administering to a subject in need of treatment a therapeutically effective amount of a compound that has peripherally-restricted 5-HT₃ receptor antagonist activity. Such compounds possess enhanced therapeutic profiles as compared with existing compounds used for treatment of 5-HT₃ mediated disorders. More specifically, by restricting the access of 5-HT₃ receptor antagonists to receptors located outside the CNS, centrally-mediated side effects can be reduced or eliminated while preserving the compounds' peripherally-mediated prokinetic attributes. Without being bound in theory, it is postulated that peripherally restricted 5-HT₃ receptor antagonists will provide a better side effect profile as compared to corresponding CNS penetrant compounds.

Definitions

These and other embodiments of the invention will be described with reference to following definitions that, for convenience, are collected here.

The language “5-HT₃ disorders” is descriptive of a disease, condition, or disorder related to 5-HT₃ in that it is effectively treated by antagonism of 5-HT₃ receptors in a subject in need of treatment. In one embodiment, the 5-HT₃ mediated disorder is a functional bowel disorder, e.g., irritable bowel syndrome (IBS). In an exemplary embodiment, the 5-HT₃ mediated disorder is diarrhea-predominant irritable bowel syndrome (IBS-d). In another embodiment, the 5-HT₃ mediated disorder is a lower urinary tract disorder, e.g., overactive bladder (OAB) including urge incontinence, or stress urinary incontinence. In another embodiment, the 5-HT₃ mediated disorder is nausea, vomiting/emesis, or retching. In an exemplary embodiment, the 5-HT₃ mediated disorder is chronic functional vomiting (CFV). In another embodiment, the 5-HT₃ mediated disorder is pain. In another embodiment, the 5-HT₃ mediated disorder is a depressive condition. In another embodiment, the 5-HT₃ mediated disorder is selected from the group consisting of obesity and weight gain, eating disorders, pre-menstrual syndrome, fibromyalgia, migraine, Parkinson's disease, stroke, schizophrenia, obsessive-compulsive disorder, fatigue, and any combination thereof. In a particular embodiment, the 5-HT₃ mediated disorder is selected from the group consisting of any one of the above-noted 5-HT₃ mediated disorders, or a combination thereof. In certain embodiments, the antagonism may account for greater than 10%, e.g., greater than 20%, e.g., greater than 30%, e.g., greater than 40%, e.g., greater than 50%, of the treatment, prevention, or reduction of the symptoms of the disorder.

The term “treatment,” as used herein, is defined as the application or administration of a therapeutic agent, i.e., a compound of the invention, to a subject, or application or administration of a therapeutic agent to an isolated tissue or cell line from a subject, who has a 5-HT₃ mediated disorder, a symptom of a 5-HT₃ mediated disorder or a predisposition toward a 5-HT₃ mediated disorder, with the purpose to cure, heal, alleviate, relieve, alter, remedy, ameliorate, improve or affect the 5-HT₃ mediated disorder, the symptoms of the 5-HT₃ mediated disorder or the predisposition toward a 5-HT₃ mediated disorder. Such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with compounds of the invention according to that individual's drug response genotype. Pharmacogenomics allows a clinician or physician to target prophylactic or therapeutic treatments to patients who will most benefit from the treatment and to avoid treatment of patients who will experience toxic drug-related side effects.

As used herein, the term “bioavailability” refers to the amount or percent of a compound, agent or drug entering the systemic circulation after administration of a given dosage form. In exemplary embodiments, bioavailability is determined as the ratio of the amount of compound, agent or drug “absorbed” from a test formulation (e.g., an oral formulation) to the amount “absorbed” after administration of a standard formulation (e.g., an aqueous formulation of the drug, given intravenously).

The amount of drug absorbed is taken as a measure of the ability of the formulation to release drug for uptake and the ability of the drug to cross from the lumen into the tissue, depending on such factors as disintegration and dissolution properties of the dosage form, and the rate of biotransformation relative to rate of absorption. Dosage forms containing identical amounts of active drug may differ markedly in their abilities to make drug available, and therefore, in their abilities to permit the drug to manifest its expected pharmacodynamic and therapeutic properties.

“Amount absorbed” is conventionally measured by one of two criteria, either the “area under the time-plasma concentration curve” (AUC) or the total (cumulative) amount of drug excreted in the urine following drug administration. A linear relationship exists between “area under the curve” and dose when the fraction of drug absorbed is independent of dose, and elimination rate (half-life) and volume of distribution are independent of dose and dosage form. A linearity of the relationship between area under the curve and dose may occur if, for example, the absorption process is a saturable one, or if drug fails to reach the systemic circulation because of, e.g., binding of drug in the intestine or biotransformation in the liver during the drug's first transit through the portal system.

The language “peripherally restricted compound,” includes compounds comprising at least one peripherally-restricting moiety or compounds that are selected based on their peripheral restriction, as well as those compounds that are formulated to restrict binding to the peripheral nervous system.

As used herein, the term “peripherally-restricting moiety” refers to a moiety that reduces the ability or prevents a compound from crossing the blood-brain barrier into the central nervous system. A peripherally-restricting moiety is typically characterized by an ionic charge. Examples of such moieties include, but are not limited to, quaternary ammonium salts, the salt of a carboxylic acid, the salt of a sulfonic acid, or the salt of a phosphoric acid. In certain embodiments, a peripherally restricted compound containing a peripherally-restricting moiety that retains residual or moderate CNS penetration, as indicated by the PSR, reduces at least one CNS side-effect as compared with the compound without the peripherally-restricting moiety.

In one embodiment, the 5-HT₃ receptor antagonists of the invention are more restricted in their action to the periphery than the parent, e.g., non-quaternary 5-HT₃ receptor antagonists, from which they were derived. The 5-HT₃ receptor antagonists of the invention do not readily cross the blood brain barrier (BBB), enter the central nervous system less readily, and therefore, are more restricted to the periphery in their action. In particular embodiments, less than 10% of the 5-HT₃ receptor antagonist penetrates or crosses the BBB. Characterization of this penetrance may be made by art-recognized analysis of BBB penetrance, e.g., as described in Example 3, wherein concentrations of the antagonist in the blood are compared with those obtained from sampling within the BBB. Although, it should be understood that such peripherally restricted compounds may be effective against 5-HT₃ receptor sites beyond the BBB by administration directly into the central nervous system.

In one embodiment, the bioavailability of the 5-HT₃ receptor antagonists of the invention is lower than the parent, e.g., non-quaternary 5-HT₃ receptor antagonists, from which they were derived. Drug bioavailability, i.e., the extent to which, and sometimes rate at which, the active moiety (drug or metabolite) enters systemic circulation, thereby gaining access to the site of action is largely determined by the dosage form of a drug. In one embodiment, the 5-HT₃ receptor antagonists of the invention are (when compared to the parent, non-quaternary 5-HT₃ antagonists from which they were derived) more slowly absorbed by the gut and/or more water soluble. In certain embodiments, the 5-HT₃ receptor antagonists of the invention act locally on the gut and do not readily enter the systemic circulation. This reduced systemic exposure, in turn, reduces the amount of compound that is absorbed into the blood stream and thus reduces exposure of, for example, the liver (reduced hepatotoxicity), kidneys (reduced renal toxicity), heart, and vascular system to the 5-HT₃ receptor antagonists, with concurrent increased concentration in the GI tract. Lower bioavailability also reduces the amount of compound that ultimately crosses the BBB. In certain embodiments, this lower bioavailability is advantageous in the treatment of several 5-HT₃ receptor-mediated conditions, for example, in certain gastrointestinal disorders, such as heartburn and IBS.

In certain embodiments, the peripherally-restricted antagonist localizes in the gastrointestinal tract, e.g., in the upper GI tract, the middle GI tract, the lower GI tract, or combination thereof. The total amount of localization may be dependent upon the particular 5-HT₃ receptor antagonist, as well as the formulation or method of administration. As such, the amount GI localization may range from zero localization to partial localization to complete localization, and may be expressed as a percentage of the total amount of antagonist administered to a subject as well as an amount of time that localization occurs. For example, GI localization may be used to describe a compound, which, upon administration (1) remains in the GI tract until completely metabolized, or until eventual excretion, or (2) 50% of the compound localizes in the GI tract for 4 hours. In particular embodiments, less than 10% of the compound crosses the gastrointestinal membrane before the compound is metabolized.

Moreover, it should be understood that this restriction to the gastrointestinal tract may exist in combination with a reduction in the ability of the compound to cross the blood brain barrier. Additionally, it should be understood that such peripherally restricted compounds may be effective against 5-HT₃ receptor sites beyond the gastrointestinal membrane by administration directly into the periphery beyond the gastrointestinal membrane, e.g., directly to the site of desired action.

Compounds of the invention that are peripherally restricted include compounds that contain at least one peripherally-restricting moiety, as well as compounds that may be peripherally restricted through the combination with additional agents based on an interaction between the additional agent and the compound. Such compounds may also selectively antagonize receptors in the periphery of the nervous system as compared with the receptors in the central nervous system.

By restricting or limiting the access of the antagonists of the invention to peripheral 5-HT₃ receptors, one or more of the 5-HT₃ antagonism pharmacological effects that are centrally-mediated, e.g., undesired CNS side effects may be reduced or alleviated while preserving the compounds' peripherally-mediated prokinetic attributes. By restricting the access of the antagonists of the invention to the gastrointestinal tract (e.g., to 5-HT₃ receptors in the GI lumen, e.g., luminal interface), one or more of the 5-HT₃ antagonism pharmacological effects that are systemically-mediated, e.g., undesired systemic side-effects (e.g., interference with other drugs that are systemically distributed, or negative effects on organs exposed upon systemic distribution of the antagonist), may be reduced or alleviated while preserving the compounds' beneficial attributes. Compounds of the invention that provide equivalent therapeutic benefit to a subject as compared with a known compound, with a reduction of the presence or intensity of side effects would be described herein as possessing an “enhanced therapeutic profile.” Furthermore, the reduction of the side-effects may allow for the administration of a greater therapeutic dose to a subject, potentially providing for “improved therapeutic effectiveness.”

The binding of the compounds to the respective receptors of the peripheral versus the central nervous system may be calculated by methods that are known to those of ordinary skill in the art and the extent of peripheral restriction, in turn, may be calculated as a ratio by using a Peripheral Restriction Ratio (PRR) defined by the formula:
[peripheral binding/central binding]=PRR

In certain embodiments, the PRR is greater than 2, e.g., greater than 3, e.g., greater than 5, e.g., greater than 10, e.g., greater than 20. In certain embodiments, a peripherally restricted compound containing a peripherally-restricting moiety that retains residual or moderate CNS penetration, as indicated by the PRR, reduces at least one CNS side-effect as compared with the compound without the peripherally-restricting moiety.

The term “interaction” includes both chemical interactions as well as physical interactions

The language “chemical interaction” includes, but is not limited to hydrophobic/hydrophilic, ionic (e.g., coulombic attraction/repulsion, ion-dipole, charge-transfer), chemical bonding, Van der Waals, and hydrogen bonding. The term chemical interaction is meant to be distinguished from physical interactions, such as physical friction between surfaces or adhesions due to formulation of the compounds.

The language “chemical bonding” is intended to include the formation of a covalent bond, e.g., organic covalent bond or inorganic covalent bond. Organic covalent bonds are defined to involve the formation of a covalent bond between the common elements of organic chemistry including but not limited to hydrogen, boron, carbon, nitrogen, oxygen, silicon, phosphorus, sulfur, and the halogens.

The term “subject,” includes living organisms in which a 5-HT₃ mediated disorder can occur, or which are susceptible to 5-HT₃ mediated disorder. Examples include animals such as mammals, including, but not limited to, primates (e.g., humans), cows, sheep, goats, horses, pigs, dogs, cats, rabbits, guinea pigs, rats, mice or other bovine, ovine, equine, canine, feline, rodent, murine species, or transgenic species thereof. In particular embodiments, the subject is human, e.g., the 5-HT₃ antagonist is pre-selected for its peripheral restriction in humans. In a particular embodiment, the species is not dog.

In certain embodiments of the invention, the subject is in need of treatment by the methods of the invention, and is selected for treatment based on this need. A subject in need of treatment is art-recognized, and includes subjects that have been identified as having a disease or disorder related to 5-HT₃, having a symptom of such a disease or disorder, or at risk of such a disease or disorder, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

In particular embodiment, the subject is in need of treatment by the peripherally restricted 5-HT₃ antagonists of the invention, and is selected for treatment based on this need. In another particular embodiment, the subject is in need of treatment by the peripherally restricted 5-HT₃ antagonists of the invention and a pre-determined additional agent, and is selected for treatment based on this need.

As used herein, the term “pharmaceutically acceptable anion” or “A⁻” is an anionic counterion of a quaternary ammonium moiety, e.g., of Formula III. In one embodiment, the pharmaceutically acceptable anion may be the product of the original quaternization reaction or may be the product of further ion exchange, i.e., creating a different salt after quaternization has occurred. For example, in one embodiment, the leaving group, I⁻, of the quaternizing group, methyl iodide, becomes the pharmaceutically acceptable anion. In another embodiment, the pharmaceutically acceptable anion may be an internal anion, i.e., wherein the quaternary ammonium moiety and the anion are covalently bonded to the same molecule (i.e., a zwitterion), e.g., O⁻.

Further examples of pharmaceutically acceptable anions included, but are not limited to, Cl⁻, Br⁻I⁻, and the carboxylates or sulfonates of appropriate organic acids that may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. In certain embodiments, the pharmaceutically acceptable anion may be selected based on the desired properties of the 5-HT₃ receptor antagonist, e.g., enhanced solubility.

As used herein, therapeutically effective amount refers to an amount sufficient to elicit the desired biological response. In the present invention the desired biological response is a reduction (complete or partial) of at least one symptom associated with the 5-HT₃ mediated disorder(s) being treated, which may be either affected by the direct mediation of the 5-HT₃ mediated disorder, i.e., the antagonism of the peripheral 5-HT₃ receptors directly resulting in the reduction of the symptom, or may be affected indirectly by association with the 5-HT₃ mediated disorder as a secondary beneficial effect of treating the 5-HT₃ mediated disorder, e.g., depressive states resulting from the existence of 5-HT₃ mediated disorder would be alleviated once the primary 5-HT₃ mediated disorder is treated or reduced.

For example, when the 5-HT₃ mediated disorder is a functional bowel disorder, for example IBS, e.g., IBS-d, a reduction in the pain or discomfort associated with IBS, as well as the reduction of the depressive state associated with one who afflicted with the symptoms of IBS, are each considered a desired biological response, wherein the first is a direct benefit and the second is an indirect benefit. As with any treatment, particularly treatment of a multi-symptom disorder, e.g., IBS, it is advantageous to treat as many disorder-related symptoms which the subject experiences. As such, when the subject is being treated for IBS a reduction in the pain or discomfort associated with IBS and a reduction in at least one other symptom of IBS selected from abnormal stool frequency, abnormal stool form, abnormal stool passage, passage of mucus and bloating or feeling of abdominal distension is preferred.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, preventing, or reducing one or more 5-HT₃ mediated disorders in a subject.

Methods of the Invention

In one embodiment, the invention is directed to a method for treating, preventing or reducing one or more 5-HT₃ mediated disorders in a subject in need thereof comprising administering to said subject a therapeutically effective amount of a quaternary ammonium derivative of MCI-225 (MCI-225-QUAT). In one embodiment, the 5-HT₃ mediated disorder is a functional bowel disorder, for example, IBS. In an exemplary embodiment, the 5-HT₃ mediated disorder is diarrhea-predominant irritable bowel syndrome (IBS-d). In another embodiment, the 5-HT₃ mediated disorder is a lower urinary tract disorder, e.g., overactive bladder (OAB) (e.g., including urge incontinence), or stress urinary incontinence. In another embodiment, the 5-HT₃ mediated disorder is nausea, vomiting/emesis, or retching. In an exemplary embodiment, the 5-HT₃ mediated disorder is chronic functional vomiting (CFV). In another embodiment, the 5-HT₃ mediated disorder is pain. In another embodiment, the 5-HT₃ mediated disorder is a depressive condition. In another embodiment, the 5-HT₃ mediated disorder is selected from the group consisting of obesity and weight gain, eating disorders, pre-menstrual syndrome, fibromyalgia, migraine, Parkinson's disease, stroke, schizophrenia, obsessive-compulsive disorder, fatigue, and any combination thereof. In a particular embodiment, the 5-HT₃ mediated disorder is selected from the group consisting of any one of the above-noted 5-HT₃ mediated disorders, or a combination thereof.

5-HT₃ Mediated Disorders

In a particular embodiment, exemplary 5-HT₃ mediated disorders may include, but are not limited to vomiting, nausea, retching, functional bowel disorders, IBS, diseases and disorders of the lower urinary tract, OAB, pain, or any combination thereof.

A. Functional Bowel Disorders

Functional Bowel Disorders (FBDs) are functional gastrointestinal disorders having symptoms attributable to the mid or lower gastrointestinal tract. FBDs can include, but are not limited to Irritable Bowel Syndrome (IBS), e.g., IBS-d, dyspepsia, functional abdominal bloating, functional constipation and functional diarrhea (see, for example, Thompson et al., Gut, 45 (Suppl II):II43-II47 (1999)). Of these disorders, IBS alone accounts for up to about 3.5 million physician visits per year, and is the most common diagnosis made by gastroenterologists, accounting for about 25% of all patients (Camilleri and Choi, Aliment. Pharm. Ther., 11:3-15 (1997)). Overall, it is estimated that IBS affects up to 20% of the adult population worldwide with only 10-50% of those afflicted with IBS actually seeking medical attention. Women apparently are more often affected than men. In addition, psychological factors, for example, emotional stress or overt psychological disease, modulate and exacerbate the physiological mechanisms that operate in IBS.

Conventional treatments for IBS are based on the severity and the nature of the symptoms being experienced by the patient and whether any psychological factors are involved. Current treatment of IBS may include one or more of the following: lifestyle changes, pharmacological treatment and psychological treatment. Although pharmacologically active agents are often used to treat IBS, there is no known general treatment which is applicable to all cases of IBS. For example, pharmacologically active agents include anti-diarrheals, such as loperamide, diphenoxylate, and codeine phosphate, for diarrhea-predominant IBS; and antispasmodic agents, such as anticholinergics and smooth muscle relaxants, such as cimetropium bromide, pinaverium bromide, octilium bromide, trimebutine, and mebeverine, for diarrhea-predominant IBS and abdominal pain. Again, while the antichloinergics and smooth muscle relaxants provide some pain relief, their effects on other symptoms associated with IBS is unclear.

Central Nervous System (CNS) treatments have received attention as potential IBS therapies because of the relationship between the CNS and the neural networks within the walls of the gut, the latter of which form the Enteric Nervous System (see, e.g., Wood et al., Gut, 45 (Suppl II):II6-II16 (1999)). In the gastrointestinal tract, 5-HT₃ receptors are located on postsynaptic enteric neurons, and on intrinsic (i.e., enteric neurons) and extrinsic (i.e., dorsal root ganglion neurons) afferent sensory fibers, some of which contact the intestinal lumen. The afferent terminals convey sensory information from the distal gastrointestinal tract to the spinal cord. Antagonism of these receptors has been found to reduce visceral pain, retard colonic transit and enhance small intestinal absorption.

In fact, clinical pharmacology studies have shown that 5-HT₃ receptor antagonists slow whole gut transit time in healthy volunteers, enhance colonic compliance and reduce perception of volume based distension in patients with IBS, and retard transit through the colon in patients with symptoms of diarrhea. However, constipation and sequelae which have resulted in colonic surgery, as well as acute ischemic colitis have been significant adverse events with the use of the 5-HT₃ receptor antagonist alosetron for the treatment of IBS.

Tricyclic antidepressants, such as amitriptyline, imipramine, and doxepin, are frequently used to treat IBS. However, the undesirable side effects associated with the use of tricyclic antidepressants to treat IBS are a significant drawback for this therapy. For example, the anticholinergic properties of the tricyclic antidepressants can cause dry mouth, constipation, blurred vision, urinary retention, weight gain, hypertension and cardiac side effects, such as palpitations and arrhythmia.

Consequently, another embodiment of the invention is a method for treating a functional bowel disorder in a subject in need thereof comprising administering to said subject a therapeutically effective amount of a quaternary ammonium derivative of MCI-225 (MCI-225-QUAT). In a particular embodiment, the functional bowel disorder is diarrhea predominant irritable bowel syndrome (IBS-d). In another embodiment, the functional bowel disorder is alternating constipation/diarrhea irritable bowel syndrome. In yet another embodiment, the functional bowel disorder is nonconstipated irritable bowel syndrome.

1. Irritable Bowel Syndrome

IBS comprises a group of functional bowel disorders in which abdominal discomfort or pain is associated with defecation or change in bowel habit and with features of disordered defecation. Due to a lack of readily identifiable structural or biochemical abnormalities in IBS, the medical community has developed a consensus definition and criteria, known as the Rome II Criteria, to aid in diagnosis of IBS. Therefore, diagnosis of IBS is one of exclusion and is based on the observed symptoms in any given case. The Rome II criteria for IBS, include at least 12 weeks in the preceding 12 months, which need not be consecutive, of abdominal pain or discomfort that has two of three features:

(1) Relieved with defecation; and/or

(2) Onset associated with a change in the frequency of stools; and/or

(3) Onset associated with a change in form (appearance) of stool.

Moreover, the following symptoms cumulatively support the diagnosis of IBS: abnormal stool frequency (for research purposes “abnormal” can be defined as >3/day and <3/week); abnormal stool form (lumpy/hard or loose/watery stool); abnormal stool passage (straining, urgency, or feeling of incomplete evacuation); passage of mucus; bloating or feeling of abdominal distension. Further, subjects with IBS exhibit visceral hypersensitivity, the presence of which physiological studies have shown is the most consistent abnormality in IBS.

It is believed that the pain associated with IBS is primarily a result of this hypersensitivity of the visceral afferent nervous system. For example, patients and controls were evaluated for their pain thresholds in response to progressive distension of the sigmoid colon induced by a balloon. At the same volume of distension, the patients reported higher pain scores compared to controls. This finding has been reproduced in many studies and with the introduction of the barostat, a computerized distension device, the distension procedures have been standardized. In this regard, the two concepts of visceral hypersensitivity, hyperalgesia and allodynia, have been introduced. More specifically, hyperalgesia refers to the situation in which normal visceral sensations are experienced at lower intraluminal volumes. While for a finding of allodynia, pain or discomfort is experienced at volumes usually producing normal internal sensations (see, for example, Mayer E. A. and Gebhart, G. F., Basic and Clinical Aspects of Chronic Abdominal Pain, Vol 9, 1.sup.st ed. Amsterdam: Elsevier, 1993:3-28).

As such, IBS is a functional bowel disorder in which abdominal pain or discomfort is associated with defecation or a change in bowel habit. Therefore, IBS has elements of an intestinal motility disorder, a visceral sensation disorder, and a central nervous disorder. While the symptoms of IBS have a physiological basis, no physiological mechanism unique to IBS has been identified. In some cases, the same mechanisms that cause occasional abdominal discomfort in healthy individuals operate to produce the symptoms of IBS. The symptoms of IBS are therefore a product of quantitative differences in the motor reactivity of the intestinal tract, and increased sensitivity to stimuli or spontaneous contractions.

2. Dyspepsia

Dyspepsia, as used herein, refers to pain or discomfort centered in the upper abdomen that can also include bloating, early satiety, postprandial fullness, nausea, anorexia, heartburn, regurgitation, and burping or belching. Generally, the symptoms of dyspepsia arise from the upper luminal GI tract. Dyspepsia can be caused by a number of foods, medications, systemic disorders and diseases of the luminal GI tract.

3. Functional Abdominal Bloating

Functional abdominal bloating comprises a group of functional bowel disorders which are dominated by a feeling of abdominal fullness or bloating, and without sufficient criteria for another functional gastrointestinal disorder. Diagnostic criteria for functional abdominal bloating are at least 12 weeks, which need not be consecutive, in the preceding 12 months of:
(1) Feeling of abdominal fullness, bloating or visible distension; and
(2) Insufficient criteria for a diagnosis of functional dyspepsia, IBS, or other functional disorder.

4. Functional Constipation

Functional constipation comprises a group of functional disorders which present as persistent difficult, infrequent or seemingly incomplete defecation. The diagnostic criteria for functional constipation are at least 12 weeks, which need not be consecutive, in the preceding 12 months of two or more of:

(1) Straining in >¼ defecations;

(2) Lumpy or hard stools in >¼ defecations;

(3) Sensation of incomplete evacuation in >¼ defecations;

(4) Sensation of anorectal obstruction/blockade in >¼ defecation;

(5) Manual maneuvers to facilitate >¼ defecations (e.g., digital evacuation, support of the pelvic floor); and/or

(6) <3 defecations/week.

Loose stools are not present, and there are insufficient criteria for IBS.

5. Functional Diarrhea

Functional diarrhea is continuous or recurrent passage of loose (mushy) or watery stools without abdominal pain. The diagnostic criteria for functional diarrhea are at least 12 weeks, which need not be consecutive, in the preceding 12 months of:

(1) Liquid (mushy) or watery stools;

(2) Present >¾ of the time; and

(3) No abdominal pain.

B. Diseases and Disorders of the Lower Urinary Tract

The language “lower urinary tract disorder” describes any disorder involving the lower urinary tract, including but not limited to overactive bladder (e.g., including urge incontinence), interstitial cystitis, prostatitis, prostadynia, benign prostatic hyperplasia, and spastic and flaccid bladder. Moreover, as used herein, “lower urinary tract” refers to all parts of the urinary tract except the kidneys.

Lower urinary tract disorders are often divided into two main categories, painful and non-painful. The language “non-painful lower urinary tract disorder” includes any lower urinary tract disorder involving sensations or symptoms, including mild or general discomfort, that a patient subjectively describes as not producing or resulting in pain. The language “painful lower urinary tract disorder” includes any lower urinary tract disorder involving sensations or symptoms that a patient subjectively describes as producing or resulting in pain.

Lower urinary tract disorders affect the quality of life of millions of men and women in the United States every year. While the kidneys filter blood and produce urine, the lower urinary tract functions to store and periodically eliminate urine and includes all other parts of the urinary tract except the kidneys. Generally, the lower urinary tract includes the ureters, the urinary bladder, sphincter and the urethra.

Among the various subtypes of 5-HT receptors, 5-HT₂ and 5-HT₃ receptors are known to mediate excitatory effects on sympathetic and somatic reflexes to increase outlet resistance. Moreover, 5-HT_{2C} and 5-HT₃ receptors have also been shown to be involved in inhibition of the micturition reflex (Downie, J. W. (1999) Pharmacological manipulation of central micturition circuitry. Curr. Opin. SPNS Inves. Drugs 1:23). In fact, 5-HT₃ receptor inhibition has been shown to diminish 5-HT mediated contractions in rabbit detrusor (Khan, M. A. et al. (2000) Doxazosin modifies serotonin-mediated rabbit urinary bladder contraction. Potential clinical relevance. Urol. Res. 28:116).

Current treatments for overactive bladder include medication, diet modification, programs in bladder training, electrical stimulation, and surgery. Currently, antimuscarinics (which are members of the general class of anticholinergics) are the primary medication used for the treatment of overactive bladder. The antimuscarinic, oxbutynin, has been the mainstay of treatment for overactive bladder. However, treatment with antimuscarinics suffers from limited efficacy and side effects such as dry mouth, dry eyes, dry vagina, blurred vision, cardiac side effects, such as palpitations and arrhythmia, drowsiness, urinary retention, weight gain, hypertension and constipation, which have proven difficult for some individuals to tolerate. Other medications which have been used “off-label” for the treatment of interstitial cystitis include, for example, antidepressants, antihistamines and anticonvulsants (See, Theoharides, T. C. (2001) New agents for the medical treatment of interstitial cystitis. Exp. Opin. Invest. Drugs 10(3): 521-46). However, in view of the unknown cause of interstitial cystitis and the suggestion that the disorder is multifactorial in origin, these additional therapies have not provided adequate relief of the associated symptoms.

Consequently, another aspect of the invention is a method of treating at least one symptom of a lower urinary tract disorder in a subject in need thereof comprising administering to said subject a therapeutically effective amount of a quaternary ammonium derivative of MCI-225 (MCI-225-QUAT).

Additionally, the invention also relates to a method of treating at least one symptom of a lower urinary tract disorder in a subject in need of treatment wherein the symptom is selected from the group consisting of urinary frequency, urinary urgency, urinary urge incontinence, nocturia and enuresis. The method comprises administering to a subject in need of treatment a therapeutically effective amount of a compound that has peripherally restricted 5-HT₃ receptor antagonist activity and NorAdrenaline Reuptake Inhibitor (NARI) activity.

Lower urinary tract disorders are particularly problematic for individuals suffering from spinal cord injury. After spinal cord injury, the kidneys continue to make urine, and urine can continue to flow through the ureters and urethra because they are the subject of involuntary neural and muscular control, with the exception of conditions where bladder to smooth muscle Dyssynergia is present. By contrast, bladder and sphincter muscles are also subject to voluntary neural and muscular control, meaning that descending input from the brain through the spinal cord drives bladder and sphincter muscles to completely empty the bladder. Following spinal cord injury, such descending input may be disrupted such that individuals may no longer have voluntary control of their bladder and sphincter muscles. Spinal cord injuries can also disrupt sensory signals that ascend to the brain, preventing such individuals from being able to feel the urge to urinate when their bladder is full.

In particular, following spinal cord injury, the bladder is usually affected in one of two ways. The first is a condition called “spastic” or “reflex” bladder, in which the bladder fills with urine and a reflex automatically triggers the bladder to empty. This usually occurs when the injury is above the T12 vertebrate level. Individuals with spastic bladder are unable to determine when, or if, the bladder will empty. The second is “flaccid” or “non-reflex” bladder, in which the reflexes of the bladder muscles are absent or slowed. This usually occurs when the injury is below the T12/L1 level. Individuals with flaccid bladder may experience over-distended or stretched bladders and “reflux” of urine through the ureters into the kidneys. Treatment options for these disorders usually include intermittent catheterization, indwelling catheterization, or condom catheterization, but these methods are invasive and frequently inconvenient. As such, “spastic bladder” or “reflex bladder” is used herein in its conventional sense to refer to a condition following spinal cord injury in which bladder emptying has become unpredictable. In addition, “flaccid bladder” or “non-reflex bladder” is used in its conventional sense to refer to a condition following spinal cord injury in which the reflexes of the bladder muscles are absent or slowed.

Urinary sphincter muscles may also be affected by spinal cord injuries, resulting in a condition known as “dyssynergia.” Dyssynergia involves an inability of urinary sphincter muscles to relax when the bladder contracts, including active contraction in response to bladder contraction, which prevents urine from flowing through the urethra and results in the incomplete emptying of the bladder and “reflux” of urine into the kidneys. Traditional treatments for dyssynergia include medications that have been somewhat inconsistent in their efficacy or surgery.

As used herein, “urinary frequency” refers to a condition in which urination occurs more frequently than the patient desires. As there is considerable interpersonal variation in the number of times in a day that an individual would normally expect to urinate, “more frequently than the patient desires” is further defined as a greater number of times per day than that patient's historical baseline. “Historical baseline” is further defined as the median number of times the patient urinated per day during a normal or desirable time period.

As used herein, “urinary urgency” refers to sudden strong urges to urinate with little or no chance to postpone the urination.

The term “incontinence” is descriptive of the inability to control excretory functions, including defecation (fecal incontinence) and urination (urinary incontinence). In certain embodiments, the urinary incontinence is stress urinary incontinence.

The language “urge incontinence” includes the involuntary loss of excreted matter associated with an abrupt and strong desire to void. As used herein, “urinary urge incontinence” (also referred to as urge incontinence) refers to the involuntary loss of urine associated with urinary urgency.

As used herein, “stress incontinence” or “stress urinary incontinence” refers to a medical condition in which urine leaks when a person coughs, sneezes, laughs, exercises, lifts heavy objects, or does anything that puts pressure on the bladder, i.e., independent of whether the person feels an urge or desire to void.

As used herein, “nocturia” refers to being awakened from sleep to urinate more frequently than the patient desires.

As used herein, “enuresis” refers to involuntary voiding of urine which can be complete or incomplete. “Nocturnal enuresis” refers to enuresis which occurs during sleep. Diurnal enuresis refers to enuresis which occurs while awake.

1. Interstitial Cystitis

Interstitial cystitis is another lower urinary tract disorder of unknown etiology that predominantly affects young and middle-aged females, although men and children can also be affected. Symptoms of interstitial cystitis can include irritative voiding symptoms, urinary frequency, urinary urgency, nocturia or suprapubic or pelvic pain related to and relieved by voiding. Many interstitial cystitis patients also experience headaches as well as gastrointestinal and skin problems. In some cases, interstitial cystitis can also be associated with ulcers or scars of the bladder. (Metts, J. F. (2001) Interstitial Cystitis: Urgency and Frequency Syndrome. American Family Physician 64(7): 1199-1206).

The language “interstitial cystitis” as used herein, is used in its conventional sense to refer to a disorder associated with symptoms that include irritative voiding symptoms, urinary frequency, urgency, nocturia, and suprapubic or pelvic pain related to and relieved by voiding.

2. Prostatitis and Prostadynia

Prostatitis and prostadynia are other lower urinary tract disorders that have been suggested to affect approximately 2-9% of the adult male population (Collins M. M. et al., (1998) “How common is prostatitis? A national survey of physician visits,” Journal of Urology, 159: 1224-1228). Prostatitis is an inflammation of the prostate, and includes bacterial prostatitis (acute and chronic) and non-bacterial prostatitis. Acute and chronic bacterial prostatitis are characterized by inflammation of the prostate and bacterial infection of the prostate gland, usually associated with symptoms of pain, urinary frequency and/or urinary urgency. Chronic bacterial prostatitis is thought to arise, e.g., from bacterial infection and is generally associated with such symptoms as inflammation of the prostate, the presence of white blood cells in prostatic fluid, and/or pain. Chronic bacterial prostatitis is distinguished from acute bacterial prostatitis based on the recurrent nature of the disorder. Chronic non-bacterial prostatitis is characterized by inflammation of the prostate which is of unknown etiology accompanied by the presence of an excessive amount of inflammatory cells in prostatic secretions not currently associated with bacterial infection of the prostate gland, and usually associated with symptoms of pain, urinary frequency and/or urinary urgency.

Currently, there are no established treatments for prostatitis and prostadynia. Antibiotics are often prescribed, but with little evidence of efficacy. COX-2 selective inhibitors and α-adrenergic blockers and have been suggested as treatments, but their efficacy has not been established. Hot sitz baths and anticholinergic drugs have also been employed to provide some symptomatic relief.

As used herein, prostatitis refers to any type of disorder associated with inflammation of the prostate, including chronic and acute bacterial prostatitis and chronic non-bacterial prostatitis, and which is usually associated with symptoms of urinary frequency and/or urinary urgency. The language “non-painful prostatitis” includes prostatitis involving sensations or symptoms, including mild or general discomfort, which a patient subjectively describes as not producing or resulting in pain. The language “painful prostatitis” includes prostatitis involving sensations or symptoms that a patient subjectively describes as producing or resulting in pain.

The language “chronic bacterial prostatitis” as used herein, is used in its conventional sense to refer to a disorder associated with symptoms that include inflammation of the prostate and positive bacterial cultures of urine and prostatic secretions. “Chronic non-bacterial prostatitis” is also used in its conventional sense to refer to a disorder associated with symptoms that include inflammation of the prostate and negative bacterial cultures of urine and prostatic secretions.

Prostadynia (chronic pelvic pain syndrome) is a disorder which mimics the symptoms of prostatitis absent inflammation of the prostate, bacterial infection of the prostate and elevated levels inflammatory cells in prostatic secretions. Prostadynia can be associated with symptoms of pain, urinary frequency and/or urinary urgency. As such, the term “prostadynia” is used in its conventional sense to refer to a disorder generally associated with painful symptoms of chronic non-bacterial prostatitis as defined above, without inflammation of the prostate.

3. Benign Prostatic Hyperplasia (BPH)

Benign prostatic hyperplasia (BPH) is a non-malignant enlargement of the prostate that is very common in men over 40 years of age. BPH is thought to be due to excessive cellular growth of both glandular and stromal elements of the prostate. The language “benign prostatic hyperplasia” as used herein, is used in its conventional sense to refer to a disorder associated with benign enlargement of the prostate gland. Symptoms of BPH can include urinary frequency, urinary urgency, urge incontinence, nocturia, or reduced urinary force and speed of flow.

Invasive treatments for BPH include transurethral resection of the prostate, transurethral incision of the prostate, balloon dilation of the prostate, prostatic stents, microwave therapy, laser prostatectomy, transrectal high-intensity focused ultrasound therapy and transurethral needle ablation of the prostate. However, complications can arise through the use of some of these treatments, including retrograde ejaculation, impotence, postoperative urinary tract infection and some urinary incontinence. Non-invasive treatments for BPH include androgen deprivation therapy and the use of 5α-reductase inhibitors and α-adrenergic blockers. However, these treatments have proven only minimally to moderately effective for some patients.

4. Overactive Bladder

Overactive bladder is a chronic medical condition that is estimated to affect 17 to 20 million people in the United States. Symptoms of overactive bladder can include urinary frequency, urinary urgency, urinary urge incontinence (accidental loss of urine) due to a sudden and unstoppable need to urinate, nocturia (the disturbance of nighttime sleep because of the need to urinate) or enuresis, resulting from overactivity of the detrusor muscle (the smooth muscle of the bladder which contracts and causes it to empty). Overactive bladder can be neurogenic or non-neurogenic.

Neurogenic overactive bladder (or neurogenic bladder) is a type of overactive bladder which occurs as a result of detrusor muscle overactivity referred to as detrusor hyperreflexia, secondary to known neurologic disorders. Patients with neurologic disorders, such as stroke, Parkinson's disease, diabetes, multiple sclerosis, peripheral neuropathy, or spinal cord lesions often suffer from neurogenic overactive bladder. In contrast, non-neurogenic overactive bladder occurs as a result of detrusor muscle overactivity referred to as detrusor muscle instability. Detrusor muscle instability can arise from non-neurological abnormalities, such as bladder stones, muscle disease, urinary tract infection or drug side effects, or can be idiopathic.

Normally, a coordinated activity between smooth muscle of the urinary bladder and striated muscle of the urethral sphincter controls micturition. The nerves that control these muscles allow a switching between storage and elimination of urine. Generally, the smooth muscle of the urinary bladder includes stretch receptors, which are responsible for the sensory reaction, or the need to urinate. For example, the bladder stretch receptors may be responsible for the urge that wakes one during nocturia. The striated muscle of the urethral sphincter, on the other hand, is generally responsible for involuntary loss of urine due to, e.g., coughing or sneezing. For example and in contrast to nocturia, control of the sphincter would be responsible for the loss of urine in nocturnal enuresis. Thus, control of the bladder stretch receptors is associated with urge urinary incontinence and control of the urethral sphincter is associated with stress urinary incontinence.

Due to the enormous complexity of micturition (the act of urination) an exact mechanism which causes overactive bladder is not known. Overactive bladder can result from hypersensitivity of sensory neurons of the urinary bladder, arising from various factors including inflammatory conditions, hormonal imbalances, and prostate hypertrophy. Destruction of the sensory nerve fibers, either from a crushing injury to the sacral region of the spinal cord, or from a disease that causes damage to the dorsal root fibers as they enter the spinal cord can also lead to overactive bladder. In addition, damage to the spinal cord or brain stem causing interruption of transmitted signals can lead to abnormalities in micturition.

Viscerosensory information from the bladder and somatosensory information from the pelvic region is relayed by nociceptive Aδ and C fibers that enter the spinal cord via the dorsal root ganglion (DRG) and project to the brainstem and thalamus via second or third order neurons (Andersson (2002) Urology 59:18-24; Andersson (2002) Urology 59:43-50; Morrison, J., Steers, W. D., Brading, A., Blok, B., Fry, C., de Groat, W. C., Kakizaki, H., Levin, R., and Thor, K. B., “Basic Urological Sciences” In: Incontinence (vol. 2) Abrams, P. Khoury, S., and Wein, A. (Eds.) Health Publications, Ltd., Plymbridge Ditributors, Ltd., Plymouth, UK., (2002). A number of different subtypes of sensory afferent neurons can be involved in neurotransmission from the lower urinary tract. These can be classified as, but not limited to, small diameter, medium diameter, large diameter, myelinated, unmyelinated, sacral, lumbar, peptidergic, non-peptidergic, IB4 positive, IB4 negative, C fiber, Aδ fiber, high threshold or low threshold neurons. Nociceptive input to the DRG is thought to be conveyed to the brain along several ascending pathways, including the spinothalamic, spinoreticular, spinomesencephalic, spinocervical, and in some cases dorsal column/medial lemniscal tracts (A. I. Basbaum and T. M. Jessell (2000) The perception of pain. In Principles of Neural Science, 4th. ed.).

While the use of gabapentin, pregabalin, and GABA analogs have been suggested as possible treatments for incontinence (see, e.g., WO00/061135), overactive bladder (or OAB) can occur with, e.g., urge incontinence, or without incontinence. In recent years, it has been recognized among those of skill in the art that the cardinal symptom of OAB is urgency without regard to any demonstrable loss of urine. For example, a recent study examined the impact of all OAB symptoms on the quality of life of a community-based sample of the United States population. (Liberman et al. (2001) Urology 57: 1044-1050). This study demonstrated that individuals suffering from OAB without any demonstrable loss of urine have an impaired quality of life when compared with controls. Additionally, individuals with urgency alone have an impaired quality of life compared with controls.

Although urgency is now believed to be the primary symptom of OAB, to date it has not been evaluated in a quantified way in clinical studies. Corresponding to this new understanding of OAB, however, the terms OAB Wet (with incontinence) and OAB Dry (without incontinence) have been proposed to describe these different patient populations (see, e.g., WO03/051354). The prevalence of OAB Wet and OAB Dry is reported to be similar in men and women, with a prevalence rate in the United States of 16.6% (Stewart et al., “Prevalence of Overactive Bladder in the United States: Results from the NOBLE Program,” Abstract Presented at the Second International Consultation on Incontinence, July 2001, Paris, France).

The language “overactive bladder (OAB)” refers to symptoms affecting the lower urinary tract which suggest detrusor muscle overactivity, in which the muscle contracts while the bladder is filling. Symptoms of OAB include urge to void, increased frequency of micturition or incontinence (involuntary loss of urine), where the loss of urine ranges from partial to total. The language “non-painful overactive bladder” includes any form of overactive bladder, as defined above, involving sensations or symptoms, including mild or general discomfort, which a patient subjectively describes as not producing or resulting in pain. Non-painful symptoms can include, but are not limited to, urinary urgency, urge incontinence, urinary frequency, and nocturia.

The language “OAB wet” is used herein to describe overactive bladder in patients with incontinence, while the language “OAB dry” is used herein to describe overactive bladder in patients without incontinence.

The language “neurogenic bladder” or “neurogenic overactive bladder” describes the condition of an overactive bladder that occurs as the result of neurological damage due to disorders including but not limited to stroke, Parkinson's disease, diabetes, multiple sclerosis, peripheral neuropathy, or spinal cord lesions.

The language “detrusor hyperreflexia” describes a condition characterized by uninhibited detrusor, wherein the patient has some sort of neurologic impairment. The language “detrusor instability” or “unstable detrusor” is descriptive of a condition where there is no neurologic abnormality.

C. Vomiting, Nausea and Retching

The act of vomiting, or emesis, can be described as the forceful expulsion of gastrointestinal contents through the mouth brought about by the descent of the diaphragm and powerful contractions of the abdominal muscles. Emesis is usually, but not always, preceded by nausea (the unpleasant feeling that one is about to vomit). Retching or dry heaves involves the same physiological mechanisms as vomiting, but occurs against a closed glottis, which prohibits the expulsion of gastric contents.

There are a number of groups of agents that have been used clinically for the treatment of emesis. These groups include: anticholinergics, antihistamines, phenothiazines, butyrophenones, cannabinoids, benzamides, glucocorticoids, benzodiazepines and 5-HT₃ receptor antagonists. In addition, tricyclic antidepressants have also been used on a limited basis. However, the undesirable side effects, such as dystonia and akathisia, sedation, anticholinergic effect and orthostatic hypotension, euphoria, dizziness, paranoid ideation, somnolence, extrapyramidal symptoms, diarrhea, perceptual disturbances, urinary incontinence, hypotension, amnesia, dry mouth, constipation, blurred vision, urinary retention, weight gain, hypertension and cardiac side effects, such as palpitations and arrhythmia continue to be associated with the use of such therapies, and are often are a significant drawback for this therapy.

Consequently, another embodiment of the present invention is a method for treating nausea, emesis/vomiting, retching or any combination thereof in a subject in need thereof comprising administering to said subject a therapeutically effective amount of a peripherally restricted 5-HT₃ receptor antagonist or a quaternary ammonium derivative of MCI-225 (MCI-225-QUAT). In specific embodiments, the subject is a human.

Vomiting, nausea, retching or combinations thereof can be caused by a number of factors including, but not limited to, anesthetics, radiation, cancer chemotherapeutic agents, toxic agents, odors, medicines, for example, a serotonin reuptake inhibitors (e.g., a selective serotonin reuptake inhibitors (SSRI)) or a dual serotonin-norepinephrine reuptake inhibitor (SNRI), analgesics such as morphine, antibiotics and antiparasitic agents, pregnancy, and motion. The language “chemotherapeutic agents,” as used herein, include, but are not limited to, for example, alkylating agents, e.g. cyclophosphamide, carmustine, lomustine, and chlorambucil; cytotoxic antibiotics, e.g. dactinomycin, doxorubicin, mitomycin-C, and bleomycin; antimetabolites, e.g. cytarabine, methotrexate, and 5-fluorouracil; vinca alkaloids, e.g. etoposide, vinblastine, and vincristine; and others such as cisplatin, dacarbazine, procarbazine, and hydroxyurea; and combinations thereof.

In the case of vomiting, nausea, retching caused by SSRI administration (e.g., daily SSRI administration), it is common for the adverse effects to diminish upon repeated administration of the drug, i.e., the patient becomes tolerant to the nausea-inducing effects of the SSRI. Accordingly, in certain embodiments, the invention features administration of a peripherally-restricted 5-HT3 receptor antagonist on an as-needed basis, for example, prior to the induction of tolerance during a course of SSRI treatment.

Conditions which are associated with vertigo (e.g., Meniere's disease and vestibular neuronitis) can also cause nausea, vomiting, retching or any combination thereof. Headache, caused by, for example, migraine, increased intracranial pressure or cerebral vascular hemorrhage can also result in nausea, vomiting, retching or any combination thereof. In addition, certain maladies of the gastrointestinal (GI) tract, for example, cholecystitis, choledocholithiasis, intestinal obstruction, acute gastroenteritis, perforated viscus, dyspepsia resulting from, for example, gastroesophageal reflux disease, peptic ulcer disease, gastroparesis, gastric or esophageal neoplasms, infiltrative gastric disorders (e.g., Menetrier's syndrome, Crohn's disease, eosinophilic gastroenteritis, sarcoidosis and amyloidosis), gastric infections (e.g., CMV, fungal, TB and syphilis), parasites (e.g., Giardia lamblia and Strongyloides stercoralis), chronic gastric volvulus, chronic intestinal ischemia, altered gastric motility disorders and/or food intolerance or Zollinger-Ellison syndrome can result in vomiting, nausea, retching or any combination thereof. However, in some cases of vomiting, nausea, retching or any combination thereof, no etiology can be determined despite extensive diagnostic testing (e.g., cyclic vomiting syndrome).

In certain embodiments, vomiting is chronic functional vomiting (CFV). CFV is a chronic condition comprised of functional vomiting and cyclic vomiting syndrome, characterized by recurrent episodes of vomiting, nausea, and abdominal pain separated by symptom-free intervals. Accordingly, under Rome II Criteria, patients with CFV experience frequent episodes of vomiting occurring on at least three separate days in a week over three months, in conjunction with a history of three or more periods of intense, acute nausea and unremitting vomiting lasting hours to days, with intervening symptom-free intervals lasting weeks to months, in the absence of known medical and psychiatric causes. However, without wishing to be bound by theory, it is believed that CFV may be caused by the abnormal function (dysfunction) of the muscles or nerves controlling the organs of the middle and upper gastrointestinal (GI) tract.

Of significant clinical relevance is the nausea and vomiting resulting from the administration of general anesthetics (commonly referred to as, post-operative nausea and vomiting, PONV), chemotherapeutic agents and radiation therapy. In fact, the symptoms caused by the chemotherapeutic agents can be so severe that the patient refuses further treatment.

For example, three types of emesis are associated with the use of chemotherapeutic agents. The first type is acute emesis, which occurs within the first 24 hours of chemotherapy. The second type is delayed emesis which occurs 24 hours or more after chemotherapy administration. The third type is anticipatory emesis, which begins prior to the administration of chemotherapy, usually in patients whose emesis was poorly controlled during a previous chemotherapy cycle.

PONV is also an important patient problem and one that patients rate as the most distressing aspect of operative procedure, even above pain. Consequently, the need for an effective anti-emetic in this area is important. As a clinical problem PONV is troublesome and requires the presence of staff to ensure that vomitus is not regurgitated, resulting in very serious clinical sequelae. Furthermore, there are certain operative procedures where it is clinically important that patients do not vomit. For example, in ocular surgery where intra-cranial ocular pressure can increase to the extent that stitches are ruptured and the operative procedure is set back in terms of success to a marked degree.

Nausea, vomiting and retching are defined as acute when symptoms are present for less than a week. The causes of nausea, vomiting and retching of short duration are often separable from etiologies leading to more chronic symptoms. In contrast, nausea, vomiting and retching are defined as chronic when symptoms are present for over a week. For example, symptoms can be continuous or intermittent and last for months or years.

In certain embodiments, the vomiting reflex may be triggered by stimulation of chemoreceptors in the upper GI tract and mechanoreceptors in the wall of the GI tract, which are activated by both contraction and distension of the gut as well as by physical damage. A coordinating center in the central nervous system controls the emetic response, and is located in the parvicellular reticular formation in the lateral medullary region of the brain. Afferent nerves to the vomiting center arise from abdominal splanchnic and vagal nerves, vestibulo-labyrinthine receptors, the cerebral cortex and the chemoreceptor trigger zone (CTZ). The CTZ lies adjacent to the area postrema and contains chemoreceptors that sample both blood and cerebrospinal fluid for noxious or toxic substances.

Direct links exist between the emetic center and the CTZ. In particular, the CTZ is exposed to emetic stimuli of endogenous origin (e.g., hormones), as well as to stimuli of exogenous origin, such as drugs. The efferent branches of cranial nerves V, VII and IX, as well as the vagus nerve and sympathetic pathways produce the complex coordinated set of muscular contractions, cardiovascular responses and reverse peristalsis that characterize vomiting.

D. Additional 5-HT₃ Mediated Disorders

Additionally, the invention relates to methods of treating other disorders which benefit from 5-HT3 receptor antagonism. Some disorders have one or more significant peripheral components which benefit from 5-HT3 receptor antagonism. Some disorders have both peripheral and CNS components which benefit from 5-HT3 receptor antagonism, the compounds primarily treating the peripheral components. Some disorders have peripheral and/or CNS components and have CNS-mediated adverse effects or side effects. Disorders particularly suited for treatment according to the methodologies of the instant invention include those which benefit from 5-HT3 receptor antagonism in the periphery (e.g., in the peripheral nervous system) and/or GI system, optionally having adverse or unwanted effects mediated by 5-HT3 receptor activity in the CNS.

Accordingly, the invention additionally relates to a method treating pain, e.g., nociceptive or neoropathic pain, fibromyalgia and depressive conditions, obesity and weight gain, pre-menstrual syndrome, eating disorders, migraine, Parkinson's disease, stroke, schizophrenia, obsessive-compulsive disorder, fatigue, and any combination thereof. The method comprises administering to a subject in need of treatment thereof a therapeutically effective amount of a compound that has peripherally-restricted 5-HT₃ receptor antagonist activity.

Compounds of the Invention

Compounds for use in the present invention include, but are not limited to, peripherally restricted compounds, as well as compounds that are considered additional agents in the present invention that may provide peripheral restriction of a 5-HT₃ receptor antagonist based on an interaction between the additional agent (e.g., which may or may not have been independently peripherally restricted) and the 5-HT₃ receptor antagonist, and compounds that are considered additional agents in the present invention that may be coadministered with a peripherally restricted 5-HT₃ receptor antagonist, e.g., providing convenient or synergistic properties, e.g., an enhanced therapeutic profile or absence of a substantial reduction in the therapeutic effectiveness. In addition, the present invention is directed to novel compounds described herein, e.g., Formulae I, II, and III, and particular embodiments thereof.

A. Peripherally-Restricted Serotonin Receptor Antagonists

The neurotransmitter serotonin was first discovered in 1948 and has subsequently been the subject of substantial scientific research. Serotonin also referred to as 5-hydroxytryptamine (5-HT), acts both centrally and peripherally on discrete 5-HT receptors. Currently, fifteen subpopulations of serotonin receptors are recognized and delineated into seven families, 5-HT₁ through 5-HT₇, i.e., including 5-HT_{1A}, 5-HT_{1B}, 5-HT_{1D}, 5-HT_{1E}, 5-HT_{1F}, 5-HT_{1P}, 5-HT_{1S}, 5-HT_{2A}, 5-HT_{2B}, 5-HT_{2C}, 5-HT₃, 5-HT₄, 5-HT₅, 5-HT₆, and 5-HT₇. These subtypes share sequence homology and display some similarities in their specificity for particular ligands. A review of the nomenclature and classification of the 5-HT receptors can be found in Neuropharm., 33: 261-273 (1994) and Pharm. Rev., 46:157-203 (1994).

5-HT₃ receptors are ligand-gated ion channels that are extensively distributed on enteric neurons in the human gastrointestinal tract, as well as other central locations. Activation of these channels and the resulting neuronal depolarization has been found to affect the regulation of visceral pain, colonic transit and gastrointestinal secretions. Antagonism of the 5-HT₃ receptors has the potential to influence sensory and motor function in the gut. Additionally, 5-HT₃ receptors are widely distributed in the peripheral nervous system (Br. J. Pharmacol., 90:229-238 (1987) and The Peripheral Actions of 5-Hydroxytryptamine, pp. 72-99, Oxford University Press, New York, 1989).

In certain embodiments of the invention, compounds that may be useful in the present invention include peripherally-restricted serotonin receptor antagonists, e.g., derivatives of the thieno[2,3-d]pyrimidines described in U.S. Pat. App. No. 4,695,568, and U.S. patent application Ser. Nos. 10/757,364, 10/757,981, 10/817,332 and 10/617,847, the entire contents of which are incorporated herein by reference, e.g., a quaternary ammonium derivative of MCI-225 (MCI-225-QUAT), e.g., compounds of Formulae I, II, or III. In one embodiment, the peripherally-restricted serotonin receptor antagonist is not zatosetron.

As used herein, the language “peripherally 5-HT₃ receptor” is descriptive of the naturally occurring 5-HT₃ receptors in the peripheral nervous system (e.g., peripheral mammalian 5-HT₃ receptors (e.g., human peripheral 5-HT₃ receptors, or murine (e.g., rat, mouse) peripheral 5-HT₃ receptors)) and to proteins having an amino acid sequence which is the same as that of a corresponding naturally occurring 5-HT₃ receptor (e.g., recombinant proteins). The term includes receptor nucleic acids/polypeptides, which may comprise a native (i.e., a naturally occurring) sequence (including a naturally occurring allelic or polymorphic variant sequence), or encode or comprise a sequence with amino acid sequence alterations (such as insertions, deletions and/or other modifications, e.g., chemical or synthetic modifications). The receptor nucleic acid/polypeptide sequence may also be entirely synthetic, e.g., encode or comprise receptor activity.

As used herein, the term “peripherally-restricted 5-HT₃ receptor antagonist” refers to an agent (e.g., a molecule, or compound) that can inhibit 5-HT₃ receptor function in the periphery of the nervous system. For example, a peripherally-restricted 5-HT₃ receptor antagonist can inhibit binding of a ligand of a 5-HT₃ receptor to said receptor and/or inhibit a 5-HT₃ receptor-mediated response (e.g., reduce the ability of 5-HT₃ to evoke the von Bezold-Jarisch reflex).

In certain embodiments, the peripherally-restricted 5-HT₃ receptor antagonist can inhibit binding of a ligand (e.g., a natural ligand, such as serotonin (5-HT₃), or other ligand such as GR65630) to a 5-HT₃ receptor in the peripheral nervous system. In certain embodiments, the peripherally-restricted 5-HT₃ receptor antagonist can bind to a 5-HT₃ receptor in the peripheral nervous system. For example, in a particular embodiment, the peripherally-restricted 5-HT₃ receptor antagonist can bind to a peripheral 5-HT₃ receptor, thereby inhibiting the binding of a ligand to said receptor and a 5-HT₃ receptor-mediated response to ligand binding. In another embodiment, the 5-HT₃ receptor antagonist can bind to a 5-HT₃ receptor, and thereby inhibit a 5-HT₃ receptor-mediated response.

Peripherally-restricted 5-HT₃ receptor antagonists can be identified and activity assessed by any suitable method, for example, by a method which assesses the ability of a compound to inhibit radioligand binding to a 5-HT₃ receptor in the periphery (see, for example, Eguchi et al., Arzneim.-Forschung/Drug Res., 47(12): 1337-47 (1997) and G. Kilpatrick et al., Nature, 330: 746-748 (1987)) and/or by their effect on the 5-HT₃-induced von Bezold-Jarisch (B-J) reflex in the cat or rat (following the general methods described by Butler et al., Br. J. Pharmacol., 94: 397-412 (1988) and Ito et al., J. Pharmacol. Exp. Ther., 280(1): 67-72 (1997), respectively).

In a particular embodiment, peripherally-restricted 5-HT₃ receptor antagonist activity of a compound can be determined according to the method described in Eguchi et al., Arzneim.-Forschung/Drug Res., 47(12): 1337-47 (1997). Specifically, the binding assays for determination of binding to the peripherally-restricted 5-HT₃ receptor can be performed on N1E-115 mouse neuroblastoma cells (American Type Culture Collection (ATCC) Accession No. CRL-2263) in 20 mmol/L HEPES buffer (pH=7.4) containing 150 mmol/L NaCl, 0.35 mmol/L of radiolabeled ligand ([³H]GR65630) and the test compound at 6 or more concentrations at 25° C. for 60 minutes. The reaction is terminated by rapid vacuum filtration onto glass fiber filter. Radioactivity trapped on the filter is measured by scintillation spectrometry. Non-specific binding is determined using 1 μmol/L of MDL-7222 (endo-8-methyl-8-azabicyclo [3.2.1]oct-3-yl-3,5-dichlorobenzoate. IC₅₀ values are calculated by nonlinear regression analysis. The affinity constants, Ki values, are calculated from the IC₅₀ values using the Cheng-Prusoff equation.

In certain embodiments, compounds having peripherally-restricted 5-HT₃ receptor antagonist activity which is suitable for use in the invention have an affinity for 5-HT₃ receptor (Ki) of not more than about 250 times the Ki of ondansetron for 5-HT₃ receptor. This relative activity to ondansetron (Ki of test agent for 5-HT₃ receptor/Ki of ondansetron for 5-HT₃ receptor), can be determined by assaying the compound of interest and ondansetron using a suitable assay under controlled conditions, for example, conditions which differ primarily in the agent being tested. In a particular embodiment, the relative activity of the peripherally-restricted 5-HT₃ receptor antagonist activity is not more than about 200 times that of ondansetron, for example, not more than about 150 times that of ondansetron, such as not more than about 100 times that of ondansetron, for example, not more than about 50 times that of ondansetron. In another particular embodiment, the compound having peripherally-restricted 5-HT₃ receptor antagonist activity has a relative activity to ondansetron of not more than about 10. In addition, and as noted above, characterization of this penetrance may be made by art-recognized analysis of BBB penetrance, or as described in Example 3, wherein concentrations of the antagonist in the blood are compared with those obtained from sampling within the BBB.

In a particular embodiment, the compounds having peripherally-restricted 5-HT₃ receptor antagonist activity are quaternary ammonium derivatives of 4-(2-fluoro-phenyl)-6-methyl-2-piperazin-1-yl-thieno[2,3-d]pyrimidine (i.e., known in the art as MCI-225 or DDP-225), also referred to herein as an “MCI-225-QUAT.” The language “quaternary ammonium derivatives of 4-(2-fluoro-phenyl)-6-methyl-2-piperazin-1-yl-thieno[2,3-d]pyrimidine” or “MCI-225-QUAT” is used herein to refer to a 4-(2-fluoro-phenyl)-6-methyl-2-piperazin-1-yl-thieno[2,3-d]pyrimidine, or MCI-225, that is derivatized with a quaternary ammonium moiety, e.g., on any position on the 4-(2-fluoro-phenyl)-6-methyl-2-piperazin-1-yl-thieno[2,3-d]pyrimidine available for substitution or on any available position on a substituent, as described herein, such that the quaternary ammonium moiety does not substantially affect the ability of the compound to perform its function, e.g., anatagonize a peripherally-restricted 5-HT₃ receptor.

The language “quaternary ammonium moiety” is art-recognized and includes, for example, nitrogen atoms that are substituted by four substituents, i.e., any substituent, e.g., noted below, which may be used to satisfy the valency requirement of quaternary substitution of a nitrogen resulting in an ammonium ion. Such moieties may include nitrogen atoms substituted by a C₁-C₆ alkyl group, e.g., CH₃, or one or more substituents that, taken together with the nitrogen, comprise a ring structure, such as a cycloalkyl group, e.g., forming a heterocycle. In certain embodiments, two of the substituents of the quaternary ammonium moiety in conjunction with the nitrogen comprise three members of a piperazine ring. In a further particular embodiment, the remaining substituents may be independently substituted with C₁-C₆ alkyl group, e.g., CH₃, 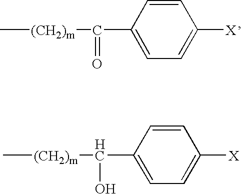
or —C(O)—NH—R₇ wherein m is an integer from about 1 to about 3, X is halogen and R₇ is a C₁-C₆ alkyl group.

In certain embodiments, the MCI-225-QUAT is represented by Formula I: 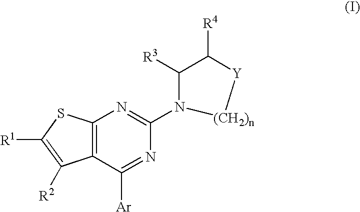
wherein R₁ and R₂ independently represent hydrogen, halogen or a C₁-C₆ alkyl group; or R₁ and R₂ together with the carbon atom to which they are attached form a cycloalkyl group having 5 to 6 carbon atoms; R₃ and R₄ independently represent hydrogen or a C₁-C₆ alkyl group; Y represents a peripherally-restricted moiety; Ar is a substituted or unsubstituted phenyl, 2-thienyl or 3-thienyl group; and n is 2 or 3; or a pharmaceutically acceptable salt thereof. In a particular embodiment, R₁ is a C₁-C₆ alkyl group and Ar is a substituted phenyl. In a specific embodiment, the substituted phenyl group is substituted with a halogen. In another particular embodiment, n is 2, R₁ is a C₁-C₆ alkyl group and Ar is phenyl substituted with fluorine. In yet another particular embodiment, n is 2, the substituted phenyl group is substituted with a halogen and R₁ is a methyl group. In yet another particular embodiment, R₂ is hydrogen. In yet another particular embodiment, Y is a quaternary ammonium salt, the salt of a carboxylic acid, the salt of a sulfonic acid, or the salt of a phosphoric acid.

In a specific embodiment of formula I, R₁ is CH₃, R₂ is H, Ar is 2-fluoro-phenylene, R₃ is H, R₄ is H, Y is N⁺(C₁₋₄)₂, and n is 2. In another preferred embodiment, Y is N⁺(CH₃)₂.

In certain embodiments, the MCI-225-QUAT is represented by formula II: 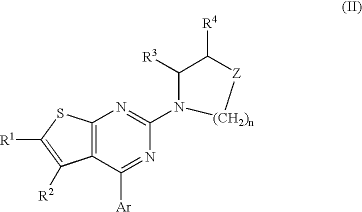
wherein R₁ and R₂ independently represent hydrogen, halogen or a C₁-C₆ alkyl group; or R₁ and R₂ together with the carbon atom to which they are attached form a cycloalkyl group having 5 to 6 carbon atoms; R₃ and R₄ independently represent hydrogen or a C₁-C₆ alkyl group; Z represents a quaternary ammonium moiety; Ar is a substituted or unsubstituted phenyl, 2-thienyl or 3-thienyl group; and n is 2 or 3; or a pharmaceutically acceptable salt thereof. In a particular embodiment, R₁ is a C₁-C₆ alkyl group and Ar is a substituted phenyl, e.g., substituted with a halogen. In another particular embodiment, n is 2, R₁ is a C₁-C₆ alkyl group and Ar is phenyl substituted with fluorine. In another particular embodiment, n is 2, the substituted phenyl group is substituted with a halogen and R₁ is a methyl group. In yet another particular embodiment, R₂ is hydrogen. In yet another particular embodiment, Z is N⁺(C₁₋₄)₂, e.g., N⁺(CH₃)₂.

In a specific embodiment of formula II, R₁ is CH₃, R₂ is H, Ar is 2-fluoro-phenylene, R₃ is H, R₄ is H, Z is N⁺(C₁₋₄)₂, and n is 2. In another preferred embodiment, Z is N⁺(CH₃)₂.

In certain embodiments, the MCI-225-QUAT is represented by formula III: 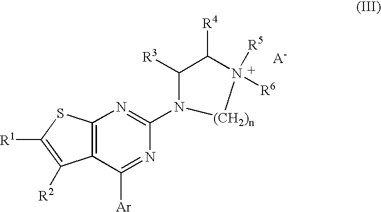
wherein R₁ and R₂ independently represent hydrogen, halogen or a C₁-C₆ alkyl group; or R₁ and R₂ together with the carbon atom to which they are attached form a cycloalkyl group having 5 to 6 carbon atoms; R₃ and R₄ independently represent hydrogen or a C₁-C₆ alkyl group; R₅ and R₆ independently represent C₁-C₆ alkyl, 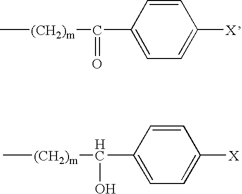
—C(O)—NH—R₇ wherein m is an integer from about 1 to about 3, X is halogen and R₇ is a C₁-C₆ alkyl group, R₅ and R₆ taken together form a cycloalkyl group, e.g., C₃-C₈, or one of R₅ and R₆ is O⁻; Ar is a substituted or unsubstituted phenyl, 2-thienyl or 3-thienyl group; A⁻ represents a pharmaceutically acceptable anion; and n is 2 or 3; or a pharmaceutically acceptable salt thereof. In a particular embodiment, R¹ is a C₁-C₆ alkyl group and Ar is a substituted phenyl, e.g., substituted with a halogen. In another particular embodiment, n is 2, R¹ is a C₁-C₆ alkyl group and Ar is phenyl substituted with fluorine. In another particular embodiment, n is 2, the substituted phenyl group is substituted with a halogen and R¹ is a methyl group. In yet another particular embodiment, R₂ is hydrogen. In another particular embodiment, R¹ is CH₃, R₂ is H, Ar is 2-fluoro-phenylene, R³ and R⁴ are H, R⁵ and R⁶ are CH₃, n is 2, and A⁻ is I⁻.

In a specific embodiment of formula III, R¹ is CH₃, R₂ is H, Ar is 2-fluoro-phenylene, R³ and R⁴ are H, R⁵ and R⁶ are CH₃, n is 2, and A⁻ is I⁻.

In certain embodiments, the MCI-225-QUAT is represented by formula IV: 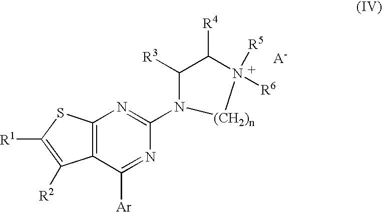
wherein R₁ and R₂ independently represent hydrogen, halogen or a C₁-C₆ alkyl group;
or R₁ and R₂ together with the carbon atom to which they are attached form a cycloalkyl group, e.g., C₃-C₈;
R₃ and R₄ independently represent hydrogen, a C₁-C₆ alkyl group, or R₃ and R₄ taken together form a cycloalkyl group, e.g., C₃-C₈, or one of R₃ and R₄ taken together with one of R₅ and R₆ form a cycloalkyl group, e.g., C₃-C₈;
R₅ and R₆ independently represent C₁-C₆ alkyl, 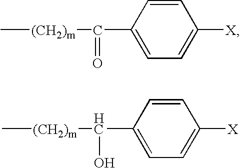
—C(O)—NH—R₇ wherein m is an integer from about 1 to about 3, X is halogen and R₇ is a C₁-C₆ alkyl group, or R₅ and R₆ taken together form a cycloalkyl group, e.g., C₃-C₈, one of R₅ and R₆ taken together with one of R₃ and R₄ form a cycloalkyl group, e.g., C₃-C₈, or one of R₅ and R₆ is O⁻; Ar is a substituted or unsubstituted phenyl, 2-thienyl or 3-thienyl group; A⁻ represents a pharmaceutically acceptable anion; and n is is 2 or 3; or a pharmaceutically acceptable salt thereof. In a particular embodiment, R₁ is a C₁-C₆ alkyl group and Ar is a substituted phenyl, e.g., substituted with a halogen. In another particular embodiment, n is 2, R₁ is a C₁-C₆ alkyl group and Ar is phenyl substituted with fluorine. In another particular embodiment, n is 2, the substituted phenyl group is substituted with a halogen and R₁ is a methyl group. In yet another particular embodiment, R₂ is hydrogen. In another particular embodiment, R¹ is CH₃, R₂ is H, Ar is 2-fluoro-phenylene, R³ and R⁴ are H, R⁵ and R⁶ are CH₃, n is 2, and A⁻ is I⁻.

In a specific embodiment of formula IV, R¹ is CH₃, R₂ is H, Ar is 2-fluoro-phenylene, R³ and R⁴ are H, R⁵ and R⁶ are CH₃, n is 2, and A⁻ is I⁻.

In another embodiment, the invention includes any novel compound or pharmaceutical compositions containing compounds of the invention described herein. For example, compounds and pharmaceutical compositions containing compounds set forth herein (e.g., Formulae I, II, and III, as well as particular embodiments thereof) are part of this invention, including salts thereof, e.g., a pharmaceutically acceptable salt.

As used herein, the language “pharmaceutically acceptable salt” refers to a salt of the administered compounds prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

Halogen refers to fluorine, chlorine, bromine or iodine.

Unless specifically indicated, the chemical groups of the present invention may be substituted or unsubstituted. Further, unless specifically indicated, the chemical substituents may in turn be substituted or unsubstituted. In addition, multiple substituents may be present on a chemical group or substituent. Examples of substituents include alkyl, alkenyl, alkynyl, aryl, halogen, hydroxyl, alkylcarbonyloxy, arylcarbonyloxy, alkoxycarbonyloxy, aryloxycarbonyloxy, carboxyl, alkylcarbonyl, arylcarbonyl, alkoxycarbonyl, aminocarbonyl, alkylaminocarbonyl, dialkylaminocarbonyl, alkylthiocarbonyl, alkoxyl, formyl, trimethylsilyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amido, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, and aromatic or heteroaromatic moieties.

In particular, “substituted” phenyl, 2-thienyl or 3-thienyl group refers to a phenyl, 2-thienyl or 3-thienyl group in which at least one of the hydrogen atoms available for substitution has been replaced with a group other than hydrogen (i.e., a substituent group). Multiple substituent groups can be present on the phenyl, 2-thienyl or 3-thienyl ring. When multiple substituents are present, the substituents can be the same or different and substitution can be at any of the substitutable sites on the ring. Substituent groups can be, for example, a halogen atom (fluorine, chlorine, bromine or iodine); an alkyl group, for example, a C₁-C₆ alkyl group such as a methyl, ethyl, propyl, butyl, pentyl or hexyl group; an alkoxy group, for example, a C₁-C₆ alkoxy group such as methoxy, ethoxy, propoxy, butoxy; a hydroxy group; a nitro group; an amino group; a cyano group; or an alkyl substituted amino group such as methylamino, ethylamino, dimethylamino or diethylamino group.

The term “alkyl” refers to saturated aliphatic groups, including straight-chain alkyl groups, branched-chain alkyl groups, cycloalkyl, heterocyclyl, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably has 20 or fewer carbon atoms in the backbone. Likewise, preferred cycloalkyls have from 3-10 carbon atoms in their ring structure, and more preferably have, 3-8 carbon atoms in the their ring structure and even more preferably have 5, 6 or 7 carbons in the ring structure.

Moreover, alkyl (e.g., methyl, ethyl, propyl, butyl, pentyl, hexyl, etc.) include both “unsubstituted alkyl” and “substituted alkyl”, the latter of which refers to alkyl moieties having substituents replacing a hydrogen on one or more carbons of the hydrocarbon backbone, which allow the molecule to perform its intended function. Examples of substituents, which are not intended to be limiting, include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₀₋₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” includes all permissible substituents of organic compounds. In a broad aspect, the permissible substituents include acyclic and cyclic, branched and unbranched, carbocyclic and heterocyclic, aromatic and nonaromatic substituents of organic compounds.

The term “aryl” includes 5- and 6-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring (e.g., phenyl, indole, thiophene) can be substituted at one or more ring positions with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. Aryl groups can also be fused or bridged with alicyclic or heterocyclic rings which are not aromatic so as to form a polycycle such as tetralin.

The terms “alkenyl” and “alkynyl” include unsaturated aliphatic groups analogous in length and possible substitution to the alkyls described above, but that contain at least one double or triple bond respectively and at least two adjacent carbon atoms.

Unless the number of carbons is otherwise specified, “lower alkyl” means an alkyl group as defined above, but having from one to ten carbons, more preferably from one to six carbon atoms in its backbone structure. Likewise, “lower alkenyl” and “lower alkynyl” have similar chain lengths (and at least two carbon atoms). Preferred alkyl groups are lower alkyls.

The terms “heterocyclyl” or “heterocyclic group” refer to 3- to 10-membered ring structures, more preferably 4- to 7-membered rings, which ring structures include one or more heteroatoms, e.g., two, three, or four. Heterocyclyl groups include pyrrolidine, oxolane, thiolane, piperidine, piperazine, morpholine, lactones, lactams such as azetidinones and pyrrolidinones, sultams, sultones, and the like. The heterocyclic ring can be substituted at one or more positions with such substituents as described above, including halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety.

The terms “polycyclyl” or “polycyclic group” refer to two or more cyclic rings (e.g., cycloalkyls, cycloalkenyls, cycloalkynyls, aryls and/or heterocyclyls) where two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety.

The term “heteroatom” as used herein means an atom of any element other than carbon or hydrogen. Preferred heteroatoms are nitrogen, oxygen, sulfur and phosphorus.

The term “aryl aldehyde,” as used herein, refers to a compound represented by the formula Ar—C(O)H, where Ar is an aryl moiety (as described above) and —C(O)H is a formyl or aldehydo group. In a preferred embodiment, the aryl aldehyde is a (substituted or unsubstituted) benzaldehyde. A variety of aryl aldehydes are commercially available, or can be prepared by routine procedures from commercially available precursors. Procedures for the preparation of aryl aldehydes include the Vilsmeier-Haack reaction (See, e.g., Jutz, Adv. Org. Chem. 9, pt. 1, 225-342 (1976)), the Gatterman reaction (Truce, Org React. 9, 37-72 (1957)), the Gatterman-Koch reaction (Crounse, Org React. 5, 290-300 (1949)), and the Reimer-Tiemann reaction (Wynberg and Meijer, Org React. 28, 1-36 (1982)).

It will be noted that the structure of some of the compounds of this invention includes asymmetric carbon atoms. It is to be understood accordingly that the isomers arising from such asymmetry (e.g., all enantiomers and diastereomers) are included within the scope of this invention unless indicated otherwise. Such isomers can be obtained in substantially pure form by classical separation techniques and by stereochemically controlled synthesis. That is, unless otherwise stipulated, any chiral carbon center may be of either (R)- or (S)-stereochemistry. Furthermore, alkenes can include either the E- or Z-geometry, where appropriate. Additionally, one skilled in the art will appreciate that the chemical structures as drawn may represent a number of possible tautomers, and the present invention also includes those tautomers.

It will further be noted that, depending upon, e.g., the methods for isolating and purifying the compounds of the present invention, there may exist a number of polymorphs of each individual compound. As used herein, the term “polymorph” refers to a solid crystalline phase of a compound represented by Formulae I, II or III, resulting from the possibility of at least two different arrangements of the molecules of the compound in the solid state. Crystalline forms of MCI-225-QUATs of the invention, e.g., Formulae I, II, or III, are of particular importance because they may be formulated in various oral unit dosage forms as for example as tablets or capsules for the treatment of 5-HT₃ mediated disorders in patients. Variations in crystal structure of a pharmaceutical drug substance may affect the dissolution, manufacturability and stability of a pharmaceutical drug product, specifically in a solid oral dosage form formulation. Therefore it may be preferred to produce MCI-225-QUATs of the invention in a pure form consisting of a single, thermodynamically stable crystal structure. It has been determined, for example, that the crystal structure of known compounds produced in accordance with commonly utilized synthesis may not be the most thermodynamically stable polymorphic form. Furthermore, it has been demonstrated that a polymorphic form may undergo conversion to a different polymorphic form when subjected to conventional manufacturing processes, such as grinding and milling. As such, certain polymorphic forms, which may not be the most thermodynamically stable form of the compound, could undergo polymorph conversion over time.

Polymorphs of a given compound will be different in crystal structure but identical in liquid or vapor states. Moreover, solubility, melting point, density, hardness, crystal shape, optical and electrical properties, vapor pressure, stability, etc., may all vary with the polymorphic form. Remington's Pharmaceutical Sciences, 18th Edition, Mack Publishing Co. (1990), Chapter 75, pages 1439-1443. Such polymorphs are also meant to be included in the scope of this invention. Varying polymorphs may be created, for example, by applying kinetic energy, e.g., by grinding, milling, or stirring, preferably at low temperature or by applying heat and subsequently cooling in a controlled manner. The compounds of the present invention may exist as a single polymorphic form or as a mixture of multiple polymorphic forms.

Furthermore, the compounds of the present invention may be suitable for silicon switching as described, e.g., in Drug Discovery Today 8(12): 551-6 (2003) “Chemistry challenges in lead optimization: silicon isoteres in drug discovery.” Briefly, it has recently been discovered that certain carbon atoms in organic compounds, such as the compounds of the present invention, may be replaced by silicon atoms without noticeable loss in activity. Accordingly, in one embodiment, the present invention is directed to an MCI-225-QUATs of the invention as described herein, e.g., defined by formulae I, II, or III, wherein one or more of the carbons in the molecule has been replaced by a silicon. The skilled artisan can readily determine which compounds are eligible for silicon switching, which carbons of such compounds may be replaced, and how to effect the switch using no more than routine experimentation found, e.g., in Drug Discovery Today 8(12): 551-6 (2003) “Chemistry challenges in lead optimization: silicon isoteres in drug discovery”, cited above.

It is understood that peripherally restricted 5-HT₃ receptor antagonists of the invention can be identified, for example, by screening libraries or collections of molecules using suitable methods. Another source for the compounds of interest is the use of combinatorial libraries that can comprise many structurally distinct molecular species. Combinatorial libraries can be used to identify lead compounds or to optimize a previously identified lead. Such libraries can be manufactured by well-known methods of combinatorial chemistry and screened by suitable methods.

Peripherally restricted 5-HT₃ receptor antagonists can also be developed (e.g., derived) from other known 5-HT₃ receptor antagonists including, but not limited to indisetron, YM-114 ((R)-2,3-dihydro-1-[(4,5,6,7-tetrahydro-1H-benzimidazol-5-yl-)carbonyl]-1H-indole), granisetron, talipexole, azasetron, bemesetron, tropisetron, ramosetron, ondansetron, palonosetron, lerisetron, alosetron, N-3389, zacopride, cilansetron, E-3620 ([3(S)-endo]-4-amino-5-chloro-N-(8-methyl-8-azabicyclo[3.2.1-]oct-3-yl-2[(1-methyl-2-butynyl)oxy]benzamide), lintopride, KAE-393, itasetron, zatosetron, dolasetron, (+/−)-zacopride, (+/−)-renzapride, (−)-YM-060, DAU-6236, BIMU-8 and GK-128 ([2-[2-methylimidazol-1-yl)methyl]-benzo[/]th-iochromen-1-one monohydrochloride hemihydrate]).

In particular embodiments, the peripherally restricted 5-HT₃ receptor antagonists is developed (e.g., derived) from a 5-HT₃ receptor antagonist selected from indisetron, granisetron, azasetron, bemesetron, tropisetron, ramosetron, ondansetron, palonosetron, lerisetron, alosetron, cilansetron, itasetron, zacopride, mirtazapine, pancopride, YM-144 (Yamanouchi) and RS17017 (Roche), and dolasetron. In one embodiment, the peripherally-restricted serotonin receptor antagonist is not zatosetron.

In another embodiment, the method comprises administering to a subject in need of treatment thereof a compound that also has NARI activity, e.g., peripherally-restricted NARI activity (i.e., described herein as a “dual serotonin-norepinephrine reuptake inhibitor (SNRI)” or “dual serotonin-noradrenaline reuptake inhibitor”).

In a preferred embodiment, compounds having either peripherally-restricted 5-HT₃ receptor activity or both peripherally-restricted 5-HT₃ receptor activity and peripherally-restricted NARI activity, such as the compounds of Formula I, II and III possess one or more characteristics selected from the group consisting of:

a) the substantial absence of anticholinergic effects;

b) the selective inhibition of noradrenaline reuptake as compared to inhibition of serotonin reuptake; and

c) the selective inhibition of noradrenaline reuptake as compared to inhibition of dopamine reuptake.

B. Coadministration of Additional Agents

In another aspect, the invention is directed to a method for treating one or more 5-HT₃ mediated disorders in a subject in need thereof comprising coadministering to said subject a peripherally-restricted 5-HT₃ receptor antagonist with an additional agent. In particular embodiments, the 5-HT₃ mediated disorder is selected from the group consisting of functional bowel disorder, for example IBS, e.g. IBS-d, symptoms of a lower urinary tract disorder, nausea, vomiting, for example CFV, retching, overactive bladder (OAB) (e.g., including urge incontinence), stress urinary incontinence, pain, fibromyalgia and depressive conditions, obesity and weight gain, pre-menstrual syndrome, eating disorders, migraine, Parkinson's disease, stroke, schizophrenia, obsessive-compulsive disorder, fatigue, and any combination thereof. The coadministration of the additional agent may be used to add functionality to peripherally restricted 5-HT₃ receptor antagonist, i.e., it may be used for treating the 5-HT₃ mediated disorder(s), an additional 5-HT₃ mediated disorder, an associated disorder, or a disorder distinct from the 5-HT₃ mediated disorder. Additionally, the peripherally restricted 5-HT₃ receptor antagonist may be used to prevent the side effects, e.g., centrally mediated side-effects, of other drugs, i.e., the coadministered additional agent (e.g., dapoxetine, which has been shown to be particularly useful for premature ejaculation (PE) in phase III clinical trials, but which patients have no tolerance to nausea side effects due to prescription in an as-needed manner.)

In certain embodiments, the additional agent is a noradrenaline reuptake inhibitor, e.g., peripherally restricted or not. In certain embodiments, the peripherally-restricted 5-HT₃ receptor antagonist is selected based on its peripheral restriction. In certain embodiments, the additional agent is selected based on its effect in combination with the peripherally-restricted 5-HT₃ receptor antagonist.

In practicing the methods of the invention, coadministration refers to administration of a first amount of a 5-HT₃ receptor antagonist compound and a second amount of an additional agent, e.g., a NARI compound (e.g., a peripherally restricted noradrenaline reuptake inhibitor or a noradrenaline reuptake inhibitor that is not restricted to the periphery), to treat one or more 5-HT3 mediated disorders. Coadministration encompasses administration of the first and second amounts of the compounds of the coadministration in an essentially simultaneous manner, such as in a single pharmaceutical composition, for example, capsule or tablet having a fixed ratio of first and second amounts, or at multiple time points, e.g., in separate capsules or tablets for each. In addition, such coadministration also encompasses use of each compound in a sequential manner, e.g., in either order (i.e., all sequences of administration are intended to be with in the scope of the present invention). When coadministration involves the separate administration of the NARI and 5-HT₃ receptor antagonist, the compounds are administered sufficiently close in time to have the desired therapeutic effect.

In another aspect, the invention is directed to a method for treating a functional bowel disorder in a subject in need thereof comprising coadministering to said subject a peripherally-restricted 5-HT₃ receptor antagonist with an additional agent.

In yet another aspect, the invention pertains to a method of treating at least one symptom of a lower urinary tract disorder in a subject in need of treatment, e.g., wherein the symptom is selected from the group consisting of urinary frequency, urinary urgency, nocturia and enuresis, comprising coadministering to said subject a peripherally-restricted 5-HT₃ receptor antagonist with an additional agent.

An additional aspect of the invention is directed to a method for treating urinary incontinence in a subject in need thereof comprising coadministering to said subject a peripherally-restricted 5-HT₃ receptor antagonist with an additional agent.

In yet another aspect, the invention pertains to a method for treating nausea, vomiting, retching or any combination thereof in a subject in need thereof comprising coadministering to said subject a peripherally-restricted 5-HT₃ receptor antagonist with an additional agent.

C. Additional Agents

Additional agents that are useful in the present invention are explicitly not intended to be limited to those additional agents recited herein. However, exemplary additional agents may provide peripheral restriction of a 5-HT₃ receptor antagonist based on an interaction between the additional agent and provide peripheral restriction of a 5-HT₃ receptor antagonist (e.g., which may or may not have been independently peripherally restricted), or may be coadministered with a peripherally restricted 5-HT₃ receptor antagonist, e.g., providing convenient or synergistic properties, e.g., an enhanced therapeutic profile or absence of a substantial reduction in the therapeutic effectiveness. Moreover, the language “an additional agent,” as used herein, is intended to be used as both singular and plural, to describe one or more additional agents, e.g., two or more additional agents coadministered with the peripherally restricted 5-HT₃ receptor antagonists may be referred to herein as “an additional agent.”

Serotonin Reuptake Inhibitor

In one embodiment, the additional agent may be a serotonin reuptake inhibitor (SRI), which may or may not be restricted to the periphery of the nervous system. SRIs can include selective serotonin reuptake inhibitors (SSRIs) such as fluoxetine, paroxetine, sertraline and the rapid onset SSRI dapoxetine. In addition, certain SSRIs are known to exhibit 5-HT_{1A} receptor activities (e.g., antagonist or partial agonist activity at the 5-HT_{1A} receptor). Compounds which have combined SSRI and 5-HT_{1A} receptor activities include those described in WO 99/02516 and WO 02/44170, the contents of which are incorporated herein by reference.

Noradrenaline/Noradrenaline Reuptake Inhibitors:

In one embodiment, the additional agent may be a NorAdrenaline Reuptake Inhibitor (NARI), which may or may not be restricted to the periphery of the nervous system (i.e., CNS-penetrant or peripherally-restricted). In exemplary embodiments, the NARI is peripherally-restricted.

As used herein, the language “NorAdrenaline Reuptake Inhibitor (NARI)” refers to an agent (e.g., a molecule, a compound) which can inhibit noradrenaline transporter function. For example, a NARI can inhibit binding of a ligand of a noradrenaline transporter to said transporter and/or inhibit transport (e.g., uptake or reuptake of noradrenaline). As such, inhibition of the noradrenaline transport function in a subject, can result in an increase in the concentration of physiologically active noradrenaline. It should be understood that NorAdrenergic Reuptake Inhibitor and NorEpinephrine Reuptake Inhibitor (NERI) are used synonymously herein with NorAdrenaline Reuptake Inhibitor (NARI).

As used herein, the language “noradrenaline transporter” refers to naturally occurring noradrenaline transporters (e.g., mammalian noradrenaline transporters (e.g., human (Homo sapiens) noradrenaline transporters, murine (e.g., rat, mouse) noradrenaline transporters)) and to proteins having an amino acid sequence which is the same as that of a corresponding naturally occurring noradrenaline transporter (e.g., recombinant proteins). The term includes transporter nucleic acids/polypeptides, which may comprise a native (i.e., a naturally occurring) sequence (including a naturally occurring allelic or polymorphic variant sequence), or encode or comprise a sequence with amino acid sequence alterations (such as insertions, deletions and/or other modifications, e.g., chemical or synthetic modifications). The transporter nucleic acid/polypeptide sequence may also be entirely synthetic, e.g., encode or comprise transporter activity.

In certain embodiments, the NARI may be defined by its ability to inhibit the binding of a ligand (e.g., a natural ligand such as noradrenaline, or other ligand such as nisoxetine) to a noradrenaline transporter. In other embodiments, the NARI may be defined by its ability to bind to a noradrenaline transporter. For example, in a particular embodiment, the NARI can bind to a noradrenaline transporter, thereby inhibiting binding of a ligand to said transporter and inhibiting transport of said ligand. In another particular embodiment, the NARI can bind to a noradrenaline transporter, and thereby inhibit transport.

The NARI activity of a compound can be determined employing suitable assays. More specifically, to determine the inhibition constant (Ki) for noradrenaline reuptake, an assay which monitors inhibition of noradrenaline (NA) uptake can be used. For example, radiolabelled noradrenaline, such as [³H]NA and the test compound of interest can be incubated under conditions suitable for uptake with brain tissue or a suitable fraction thereof, for example, a synaptosomal fraction from rat brain tissue (harvested and isolated in accordance with generally accepted techniques), and the amount of uptake of [³H]NA in the tissue or fraction can be determined (e.g., by liquid scintillation spectrometry). IC₅₀ values can be calculated by nonlinear regression analysis. The inhibition constants, Ki values, can then be calculated from the IC₅₀ values using the Cheng-Prusoff equation: 1 K i=IC₅₀ 1+([L]/K d) wherein [L]=the concentration of free radioligand used in the assay and K_{d}=the equilibrium dissociation constant of the radioligand. To determine the non-specific uptake, incubations can be performed by following the same assay, but in the absence of test compound at 4° C. (i.e., under conditions not suitable for uptake). In a particular embodiment, NARI activity is determined using the radioligand uptake assay described above, according to the procedure detailed in Eguchi et al., Arzneim.-Forschung/Drug Res., 47(12): 1337-47 (1997).

NARI compounds suitable for use in the invention have a Ki value for NARI activity of about 500 nmol/L or less, such as about 250 nmol/L or less, for example, about 100 nmol/L or less. In a particular embodiment, the Ki value for NARI activity is about 100 nmol/L or less. It is understood that the exact value of the Ki for a particular compound can vary depending on the assay conditions employed for determination (e.g., radioligand and tissue source). As such, it is convenient to employ a single assay to determine the NARI activity, e.g., according to the radioligand binding assay described in Eguchi et al., Arzneim.-Forschung/Drug Res., 47(12): 1337-47 (1997).

Selective inhibition of noradrenaline reuptake as compared to inhibition of serotonin or dopamine reuptake can be determined by comparing the Ki values for the respective reuptake inhibitions. The inhibition constants for serotonin and dopamine reuptake can be determined as described above for nordrenaline, e.g., described in Eguchi et al., however, employing the appropriate radioligand and tissue for the activity being assessed (e.g., [³H] 5-HT for serotonin, using e.g., hypothalamic or cortical tissue and [³H]DA for dopamine (DA), using e.g., striatal tissue).

Following determination of the Ki values for inhibition of noradrenaline, serotonin and/or dopamine uptake, the ratio of the activities can be determined. Selective inhibition of noradrenaline reuptake as compared to inhibition of serotonin reuptake and/or dopaminergic reuptake, refers to a compound having a Ki value for inhibition of serotonin (re)uptake and/or dopamine (re)uptake which is about 10 times or more than the Ki for inhibition of noradrenaline (re)uptake. That is, the ratio, Ki inhibition of serotonin (re)uptake/Ki inhibition of noradrenaline (re)uptake, is about 10 or more, such as about 15 or more, about 20 or more, for example, about 30, 40 or 50 or more. Likewise, the ratio, [Ki inhibition of dopamine (re)uptake]/[Ki inhibition noradrenaline (re)uptake], is about 10 or more, such as about 15 or more, e.g., about 20 or more, for example, about 30, 40, 50 or more. In a particular embodiment, the Ki values for comparison are determined according to the method of Eguchi et al., e.g., using a synaptosomal preparation from rat hypothalamic tissue, e.g., for inhibition of noradrenaline uptake and from rat striatal tissue for inhibition of dopamine uptake.

In another embodiment, the NARI is characterized by the substantial absence of anticholinergic effects. As used herein, substantial absence of anticholinergic effects, refers to a compound which has an IC₅₀ value for binding to muscarinic receptors of about 1 μmol/L or more. The IC₅₀ value for binding to muscarinic receptors can be determined using a suitable assay, such as an assay which determines the ability of a compound to inhibit the binding of suitable radioligand to muscarinic receptors, e.g., as described in Eguchi et al., Arzneim.-Forschung/Drug Res., 47(12): 1337-47 (1997).

In a particular embodiment, the NARI compound can be selected from venlafaxine, duloxetine, buproprion, milnacipran, reboxetine, lefepramine, desipramine, nortriptyline, tomoxetine, maprotiline, oxaprotiline, levoprotiline, viloxazine, nisoxetine, and atomoxetine. In a specific embodiment, the NARI compound can be selected from reboxetine, lefepramine, desipramine, nortriptyline, tomoxetine, maprotiline, oxaprotiline, levoprotiline, viloxazine and atomoxetine.

Selective inhibition of noradrenaline reuptake in the periphery of the central nervous system as compared to inhibition of serotonin or dopamine reuptake in the periphery of the central nervous system can be determined by comparing the Ki values for the respective reuptake inhibitions. The inhibition constants for serotonin and dopamine reuptake can be determined as described above for noradrenaline, but employing the appropriate radioligand and tissue for the activity being assessed (e.g., [³H] 5-HT for serotonin, using e.g., hypothalamic or cortical tissue and [³H]DA for dopamine (DA), using e.g., striatal tissue). A particular method of determining serotonin reuptake inhibition and dopaminergic reuptake inhibition is described in Eguchi et al., Arzneim-Forschung/Drug Res., 47(12): 1337-47 (1997).

Serotonin-Norepinephrine Reuptake Inhibitors (SNRI)

In one embodiment, the additional agent may be a serotonin-norepinephrine reuptake inhibitors (SNRI), which may or may not be restricted to the periphery of the nervous system.

As used herein, the term serotonin-norepinephrine reuptake inhibitors (SNRI) refers to an agent (e.g., a molecule, a compound) which can inhibit the reuptake of both serotonin and norepinephrine. As such, inhibition of the reuptake of serotonin and norepinephrine in a subject can result in an increase in the concentration of physiologically active serotonin and norepinephrine.

SNRIs, e.g., venlafaxine (Effexor®) and duloxetine, generally function to correct the imbalance of both serotonin and norepinephrine in the brain. SNRIs have been used in the treatment of Major Depression and have also been found to be effective in several other disorders, including obsessive compulsive disorder, panic disorder, social phobia and in children with Attention Deficit Hyperactivity Disorder. Duloxetine has also been shown to relieve pain associated with depression (Goldstein, DL et al., 2004 Psychosomatics 45:17-28). Additional compounds with suitable SNRI activity can be identified by the skilled artisan using no more than routine experimentation (see, e.g., Wong, D. et al 1993, Neuropsychopharmacol. 8:23-33). For example, suitable SNRIs include, but are not limited to, venlafaxine (e.g., EFFEXOR), duloxetine (e.g., CYMBALTA), amoxapine, bicifadine, maprotiline, milnacipran, and derivatives thereof.

Further Examples of Additional Agents

An additional therapeutic agent suitable for use as additional agents in the methods and pharmaceutical compositions described herein, can be, but is not limited to, for example: an anticholinergic (e.g., scopolomine); an antihistamine (e.g., dimenhydrinate and diphenhydramine); a phenothiazine (e.g., prochlorperazine and chlorpromazine); a butyrophenone (haloperidol and droperidol); a cannabinoid (e.g., tetrahydrocannabinol and nabilone); a benzamide (e.g., metoclopramide, cisapride and trimethobenzamide); a corticosteroid; a glucocorticoid (e.g., dexamethasone and methylprednisolone); a benzodiazepine (e.g., lorazepam); or any combination thereof. In a particular embodiment, the additional therapeutic agent is a glucocorticoid.

Pharmaceutical Compositions and Modes of Administration

A. Pharmaceutical Compositions

In an additional embodiment, the invention relates to a pharmaceutical composition comprising a peripherally-restricted 5-HT₃ receptor antagonist and a pharmaceutically acceptable carrier for treating one or more 5-HT₃ mediated disorders in a subject, wherein the peripherally-restricted 5-HT₃ receptor antagonist is selected based on its peripheral restriction, e.g., an MCI-225-QUAT.

Another embodiment of the invention is a pharmaceutical composition comprising a peripherally-restricted 5-HT₃ receptor antagonist, an additional agent and a pharmaceutically acceptable carrier for treating one or more 5-HT₃ mediated disorders in a subject.

In particular embodiments, the 5-HT₃ mediated disorder is selected from the group consisting of functional bowel disorder, for example, IBS, e.g., IBS-d, symptoms of a lower urinary tract disorder, nausea, vomiting, for example, CFV, retching, overactive bladder (OAB) (e.g., including urge incontinence), stress urinary incontinence, pain, fibromyalgia and depressive conditions, obesity and weight gain, pre-menstrual syndrome, eating disorders, migraine, Parkinson's disease, stroke, schizophrenia, obsessive-compulsive disorder, fatigue, and any combination thereof.

In another embodiment, the invention is directed to a pharmaceutical composition comprising a peripherally-restricted 5-HT₃ receptor antagonist and a pharmaceutically acceptable carrier for treating a functional bowel disorder, for example, IBS, e.g., IBS-d, in a subject, wherein the peripherally-restricted 5-HT₃ receptor antagonist is selected based on its peripheral restriction, e.g., an MCI-225-QUAT.

In yet another embodiment, the invention is directed to a pharmaceutical composition comprising a peripherally-restricted 5-HT₃ receptor antagonist, an additional agent and a pharmaceutically acceptable carrier for treating a functional bowel disorder, for example, IBS, e.g., IBS-d, in a subject.

Another embodiment of the invention is a pharmaceutical composition comprising a peripherally-restricted 5-HT₃ receptor antagonist and a pharmaceutically acceptable carrier for treating at least one symptom of a lower urinary tract disorder in a subject in need of treatment, wherein the symptom is selected from the group consisting of urinary frequency, urinary urgency, nocturia and enuresis, wherein the peripherally-restricted 5-HT₃ receptor antagonist is selected based on its peripheral restriction, e.g., an MCI-225-QUAT.

In an additional embodiment, the invention relates to a pharmaceutical composition comprising a peripherally-restricted 5-HT₃ receptor antagonist, an additional agent and a pharmaceutically acceptable carrier for treating at least one symptom of a lower urinary tract disorder in a subject in need of treatment, wherein the symptom is selected from the group consisting of urinary frequency, urinary urgency, nocturia and enuresis.

In another embodiment, the invention relates to a pharmaceutical composition comprising a peripherally-restricted 5-HT₃ receptor antagonist and a pharmaceutically acceptable carrier for treating urinary incontinence in a subject in need thereof, wherein the peripherally-restricted 5-HT₃ receptor antagonist is selected based on its peripheral restriction.

Another embodiment of the invention is a pharmaceutical composition comprising a peripherally-restricted 5-HT₃ receptor antagonist, an additional agent and a pharmaceutically acceptable carrier for treating urinary incontinence in a subject.

In an additional embodiment, the invention relates to a pharmaceutical composition comprising a peripherally-restricted 5-HT₃ receptor antagonist and a pharmaceutically acceptable carrier for treating nausea, vomiting, for example CFV, retching or any combination thereof in a subject, wherein the peripherally-restricted 5-HT₃ receptor antagonist is selected based on its peripheral restriction, e.g., an MCI-225-QUAT.

Another embodiment of the invention is a pharmaceutical composition comprising a peripherally-restricted 5-HT₃ receptor antagonist, an additional agent and a pharmaceutically acceptable carrier for treating nausea, vomiting, for example, CFV, retching or any combination thereof in a subject.

B. Formulations for Administration

The compounds for use in the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For example, for oral administration the compounds can be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY™ film coating systems available from Colorcon, West Point, Pa. (e.g., OPADRY™ OY Type, OY—C Type, Organic Enteric OY-P Type, Aqueous Enteric OY-A Type, OY-PM Type and OPADRY™ White, 32K18400). Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

The dosage form may also be a capsule, in which case the active agent-containing composition may be encapsulated in the form of a liquid or solid (including particulates such as granules, beads, powders or pellets). Suitable capsules can be hard or soft, and are generally made of gelatin, starch, or a cellulosic material, with gelatin capsules preferred. Two-piece hard gelatin capsules are preferably sealed, such as with gelatin bands or the like. (See, for e.g., Remington: The Science and Practice of Pharmacy, supra), which describes materials and methods for preparing encapsulated pharmaceuticals. If the active agent-containing composition is present within the capsule in liquid form, a liquid carrier can be used to dissolve the active agent(s). The carrier should be compatible with the capsule material and all components of the pharmaceutical composition, and should be suitable for ingestion.

Parenteral Administration

For parenteral administration, the compounds for use in the method of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

Transmucosal Administration

Transmucosal administration is carried out using any type of formulation or dosage unit suitable for application to mucosal tissue. For example, the selected active agent can be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

Preferred buccal dosage forms will typically comprise a therapeutically effective amount of an active agent and a bioerodible (hydrolyzable) polymeric carrier that may also serve to adhere the dosage form to the buccal mucosa. The buccal dosage unit can be fabricated so as to erode over a predetermined time period, wherein drug delivery is provided essentially throughout. The time period is typically in the range of from about 1 hour to about 72 hours. Preferred buccal delivery preferably occurs over a time period of from about 2 hours to about 24 hours. Buccal drug delivery for short term use should preferably occur over a time period of from about 2 hours to about 8 hours, more preferably over a time period of from about 3 hours to about 4 hours. As needed buccal drug delivery preferably will occur over a time period of from about 1 hour to about 12 hours, more preferably from about 2 hours to about 8 hours, most preferably from about 3 hours to about 6 hours. Sustained buccal drug delivery will preferably occur over a time period of from about 6 hours to about 72 hours, more preferably from about 12 hours to about 48 hours, most preferably from about 24 hours to about 48 hours. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver.

The amount of the active agent in the buccal dosage unit will of course depend on the potency of the agent and the intended dosage, which, in turn, is dependent on the particular individual undergoing treatment, the specific indication, and the like. The buccal dosage unit will generally contain from about 1.0 wt. % to about 60 wt. % active agent, preferably on the order of from about 1 wt. % to about 30 wt. % active agent. With regard to the bioerodible (hydrolyzable) polymeric carrier, it will be appreciated that virtually any such carrier can be used, so long as the desired drug release profile is not compromised, and the carrier is compatible with the active agents to be administered and any other components of the buccal dosage unit. Generally, the polymeric carrier comprises a hydrophilic (water-soluble and water-swellable) polymer that adheres to the wet surface of the buccal mucosa. Examples of polymeric carriers useful herein include acrylic acid polymers and co, e.g., those known as “carbomers” (Carbopol™, which may be obtained from B. F. Goodrich, is one such polymer). Other suitable polymers include, but are not limited to: hydrolyzed polyvinylalcohol; polyethylene oxides (e.g., Sentry Polyox™ water soluble resins, available from Union Carbide); polyacrylates (e.g., Gantrez™, which may be obtained from GAF); vinyl polymers and copolymers; polyvinylpyrrolidone; dextran; guar gum; pectins; starches; and cellulosic polymers such as hydroxypropyl methylcellulose, (e.g., Methocel™, which may be obtained from the Dow Chemical Company), hydroxypropyl cellulose (e.g., Klucel™, which may also be obtained from Dow), hydroxypropyl cellulose ethers (see, e.g., U.S. Pat. No. 4,704,285 to Alderman), hydroxyethyl cellulose, carboxymethyl cellulose, sodium carboxymethyl cellulose, methyl cellulose, ethyl cellulose, cellulose acetate phthalate, cellulose acetate butyrate, and the like.

Other components can also be incorporated into the buccal dosage forms described herein. The additional components include, but are not limited to, disintegrants, diluents, binders, lubricants, flavoring, colorants, preservatives, and the like. Examples of disintegrants that may be used include, but are not limited to, cross-linked polyvinylpyrrolidones, such as crospovidone (e.g., Polyplasdone™ XL, which may be obtained from GAF), cross-linked carboxylic methylcelluloses, such as croscarmelose (e.g., Ac-di-sol™, which may be obtained from FMC), alginic acid, and sodium carboxymethyl starches (e.g., Explotab™, which can be obtained from Edward Medell Co., Inc.), methylcellulose, agar bentonite and alginic acid. Suitable diluents include those which are generally useful in pharmaceutical formulations prepared using compression techniques, e.g., dicalcium phosphate dihydrate (e.g., Di-Tab™, which may be obtained from Stauffer), sugars that have been processed by cocrystallization with dextrin (e.g., co-crystallized sucrose and dextrin such as Di-Pak™, which may be obtained from Amstar), calcium phosphate, cellulose, kaolin, mannitol, sodium chloride, dry starch, powdered sugar and the like. Binders, if used, include those that enhance adhesion. Examples of such binders include, but are not limited to, starch, gelatin and sugars such as sucrose, dextrose, molasses, and lactose. Particularly preferred lubricants are stearates and stearic acid, and an optimal lubricant is magnesium stearate.

Sublingual and lingual dosage forms include tablets, creams, ointments, lozenges, pastes, and any other suitable dosage form where the active ingredient is admixed into a disintegrable matrix. The tablet, cream, ointment or paste for sublingual or lingual delivery comprises a therapeutically effective amount of the selected active agent and one or more conventional nontoxic carriers suitable for sublingual or lingual drug administration. The sublingual and lingual dosage forms of the present invention can be manufactured using conventional processes. The sublingual and lingual dosage units can be fabricated to disintegrate rapidly. The time period for complete disintegration of the dosage unit is typically in the range of from about 10 seconds to about 30 minutes, and optimally is less than 5 minutes.

Other components can also be incorporated into the sublingual and lingual dosage forms described herein. The additional components include, but are not limited to binders, disintegrants, wetting agents, lubricants, and the like. Examples of binders that can be used include water, ethanol, polyvinylpyrrolidone; starch solution gelatin solution, and the like. Suitable disintegrants include dry starch, calcium carbonate, polyoxyethylene sorbitan fatty acid esters, sodium lauryl sulfate, stearic monoglyceride, lactose, and the like. Wetting agents, if used, include glycerin, starches, and the like. Particularly preferred lubricants are stearates and polyethylene glycol. Additional components that may be incorporated into sublingual and lingual dosage forms are known, or will be apparent, to those skilled in this art (See, e.g., Remington: The Science and Practice of Pharmacy, supra).

Transurethal Administration

With regard to transurethal administration, the formulation can comprise a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials, with polyethylene glycol and derivatives thereof particularly preferred. A transurethral permeation enhancer can be included in the dosage from. Examples of suitable permeation enhancers include dimethylsulfoxide (“DMSO”), dimethyl formamide (“DMF”), N,N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone™ from Nelson Research & Development Co., Irvine, Calif.), SEPA™ (available from Macrochem Co., Lexington, Mass.), surfactants as discussed above, including, for example, Tergitol™, Nonoxynol-9™ and TWEEN-80™, and lower alkanols such as ethanol.

Transurethral drug administration, as explained in U.S. Pat. Nos. 5,242,391, 5,474,535, 5,686,093 and 5,773,020, can be carried out in a number of different ways using a variety of urethral dosage forms. For example, the drug can be introduced into the urethra from a flexible tube, squeeze bottle, pump or aerosol spray. The drug can also be contained in coatings, pellets or suppositories that are absorbed, melted or bioeroded in the urethra. In certain embodiments, the drug is included in a coating on the exterior surface of a penile insert. It is preferred, although not essential, that the drug be delivered from at least about 3 cm into the urethra, and preferably from at least about 7 cm into the urethra. Generally, delivery from at least about 3 cm to about 8 cm into the urethra will provide effective results in conjunction with the present method.

Urethral suppository formulations containing PEG or a PEG derivative can be conveniently formulated using conventional techniques, e.g., compression molding, heat molding or the like, as will be appreciated by those skilled in the art and as described in the pertinent literature and pharmaceutical texts. (See, e.g., Remington: The Science and Practice of Pharmacy, supra), which discloses typical methods of preparing pharmaceutical compositions in the form of urethral suppositories. The PEG or PEG derivative preferably has a molecular weight in the range of from about 200 to about 2,500 g/mol, more preferably in the range of from about 1,000 to about 2,000 g/mol. Suitable polyethylene glycol derivatives include polyethylene glycol fatty acid esters, for example, polyethylene glycol monostearate, polyethylene glycol sorbitan esters, e.g., polysorbates, and the like. Depending on the particular active agent, urethral suppositories may contain one or more solubilizing agents effective to increase the solubility of the active agent in the PEG or other transurethral vehicle.

It may be desirable to deliver the active agent in a urethral dosage form that provides for controlled or sustained release of the agent. In such a case, the dosage form can comprise a biocompatible, biodegradable material, typically a biodegradable polymer. Examples of such polymers include polyesters, polyalkylcyanoacrylates, polyorthoesters, polyanhydrides, albumin, gelatin and starch. As explained, for example, in PCT Publication No. WO 96/40054, these and other polymers can be used to provide biodegradable microparticles that enable controlled and sustained drug release, in turn minimizing the required dosing frequency.

The urethral dosage form will preferably comprise a suppository that is from about 2 to about 20 mm in length, preferably from about 5 to about 10 mm in length, and less than about 5 mm in width, preferably less than about 2 mm in width. The weight of the suppository will typically be in the range of from about 1 mg to about 100 mg, preferably in the range of from about 1 mg to about 50 mg. However, it will be appreciated by those skilled in the art that the size of the suppository can and will vary, depending on the potency of the drug, the nature of the formulation, and other factors.

Transurethral drug delivery may involve an “active” delivery mechanism such as iontophoresis, electroporation or phonophoresis. Devices and methods for delivering drugs in this way are well known in the art. Iontophoretically assisted drug delivery is, for example, described in PCT Publication No. WO 96/40054, cited above. Briefly, the active agent is driven through the urethral wall by means of an electric current passed from an external electrode to a second electrode contained within or affixed to a urethral probe.

Transrectal Administration

Preferred transrectal dosage forms can include rectal suppositories, creams, ointments, and liquid formulations (enemas). The suppository, cream, ointment or liquid formulation for transrectal delivery comprises a therapeutically effective amount of the selected active agent and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours. The time period for complete disintegration is preferably in the range of from about 10 minutes to about 6 hours, and optimally is less than about 3 hours.

Other components can also be incorporated into the transrectal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite.

Vaginal or Perivaginal Administration

Preferred vaginal or perivaginal dosage forms include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention can be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit can be fabricated to disintegrate rapidly or over a period of several hours. The time period for complete disintegration is preferably in the range of from about 10 minutes to about 6 hours, and optimally is less than about 3 hours.

Other components can also be incorporated into the vaginal or perivaginal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite.

Intranasal or Inhalation Administration

The active agents can also be administered intranasally or by inhalation. Compositions for intranasal administration are generally liquid formulations for administration as a spray or in the form of drops, although powder formulations for intranasal administration, e.g., insufflations, nasal gels, creams, pastes or ointments or other suitable formulators can be used. For liquid formulations, the active agent can be formulated into a solution, e.g., water or isotonic saline, buffered or unbuffered, or as a suspension. Preferably, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers can also include nasal gels, creams, pastes or ointments with a viscosity of, e.g., from about 10 to about 6500 cps, or greater, depending on the desired sustained contact with the nasal mucosal surfaces. Such carrier viscous formulations can be based upon, for example, alkylcelluloses and/or other biocompatible carriers of high viscosity well known to the art (see e.g., Remington: The Science and Practice of Pharmacy, supra). Other ingredients, such as preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation can take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations can contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation can also comprise dry powder formulations, particularly insufflations in which the powder has an average particle size of from about 0.1 μm to about 50 μm, preferably from about 1 μm to about 25 μm.

Topical Formulations

Topical formulations can be in any form suitable for application to the body surface, and may comprise, for example, an ointment, cream, gel, lotion, solution, paste or the like, and/or may be prepared so as to contain liposomes, micelles, and/or microspheres. Preferred topical formulations herein are ointments, creams and gels.

Ointments, as is well known in the art of pharmaceutical formulation, are semisolid preparations that are typically based on petrolatum or other petroleum derivatives. The specific ointment base to be used, preferably provides for optimum drug delivery, and, preferably, will provides for other desired characteristics as well, e.g., emolliency or the like. The ointment base is preferably inert, stable, nonirritating and nonsensitizing. As explained in Remington: The Science and Practice of Pharmacy, supra, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight (See, e.g., Remington: The Science and Practice of Pharmacy, supra).

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels-are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol™ trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Various additives, known to those skilled in the art, may be included in the topical formulations. For example, solubilizers may be used to solubilize certain active agents. For those drugs having an unusually low rate of permeation through the skin or mucosal tissue, it may be desirable to include a permeation enhancer in the formulation; suitable enhancers are as described elsewhere herein.

Transdermal Administration

The compounds of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active agent and any other materials that are present, the backing is preferably made of a sheet or film of a flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well known in the art and include, for example, those enhancers listed above in transmucosal compositions.

Intrathecal Administration

One common system utilized for intrathecal administration is the APT Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication can then be administered directly to cells in the spinal cord involved in conveying sensory and motor signals associated with lower urinary tract disorders.

Another system available from Medtronic that is commonly utilized for intrathecal administration is the fully implantable, programmable SynchroMed™ Infusion System. The SynchroMed™ Infusion System has two parts that are both placed in the body during a surgical procedure: the catheter and the pump. The catheter is a small, soft tube. One end is connected to the catheter port of the pump, and the other end is placed in the intrathecal space. The pump is a round metal device about one inch (2.5 cm) thick, three inches (8.5 cm) in diameter, and weighs about six ounces (205 g) that stores and releases prescribed amounts of medication directly into the intrathecal space. It can be made of titanium, a lightweight, medical-grade metal. The reservoir is the space inside the pump that holds the medication. The fill port is a raised center portion of the pump through which the pump is refilled. The doctor or a nurse inserts a needle through the patient's skin and through the fill port to fill the pump. Some pumps have a side catheter access port that allows the doctor to inject other medications or sterile solutions directly into the catheter, bypassing the pump.

The SynchroMed™ pump automatically delivers a controlled amount of medication through the catheter to the intrathecal space around the spinal cord, where it is most effective. The exact dosage, rate and timing prescribed by the doctor are entered in the pump using a programmer, an external computer-like device that controls the pump's memory. Information about the patient's prescription can be stored in the pump's memory. The doctor can easily review this information by using the programmer. The programmer communicates with the pump by radio signals that allow the doctor to tell how the pump is operating at any given time. The doctor also can use the programmer to change your medication dosage.

Methods of intrathecal administration can include those described above available from Medtronic, as well as other methods that are known to one of skill in the art.

Intravesical Administration

The term intravesical administration is used herein in its conventional sense to mean delivery of a drug directly into the bladder. Suitable methods for intravesical administration can be found in U.S. Pat. Nos. 6,207,180 and 6,039,967, for example.

Additional Administration Forms

Additional dosage forms of this invention include dosage forms as described in U.S. Pat. No. 6,340,475, U.S. Pat. No. 6,488,962, U.S. Pat. No. 6,451,808, U.S. Pat. No. 5,972,389, U.S. Pat. No. 5,582,837, and U.S. Pat. No. 5,007,790. Additional dosage forms of this invention also include dosage forms as described in U.S. patent application Ser. No. 20030147952, U.S. patent application Ser. No. 20030104062, U.S. patent application Ser. No. 20030104053, U.S. patent application Ser. No. 20030044466, U.S. patent Application Ser. No. 20030039688, and U.S. patent application Ser. No. 20020051820. Additional dosage forms of this invention also include dosage forms as described in PCT Patent Application WO 03/35041, PCT Patent Application WO 03/35040, PCT Patent Application WO 03/35029, PCT Patent Application WO 03/35177, PCT Patent Application WO 03/35039, PCT Patent Application WO 02/96404, PCT Patent Application WO 02/32416, PCT Patent Application WO 01/97783, PCT Patent Application WO 01/56544, PCT Patent Application WO 01/32217, PCT Patent Application WO 98/55107, PCT Patent Application WO 98/11879, PCT Patent Application WO 97/47285, PCT Patent Application WO 93/18755, and PCT Patent Application WO 90/11757.

For intrabronchial or intrapulmonary administration, conventional formulations can be employed.

Further, the compounds for use in the method of the invention can be formulated in a sustained release preparation, further described herein. For example, the compounds can be formulated with a suitable polymer or hydrophobic material which provides sustained and/or controlled release properties to the active agent compound. As such, the compounds for use the method of the invention can be administered in the form of microparticles for example, by injection or in the form of wafers or discs by implantation.

In one embodiment, the dosage forms of the present invention include pharmaceutical tablets for oral administration as described in U.S. patent application Ser. No. 20030104053. For example, suitable dosage forms of the present invention can combine both immediate-release and prolonged-release modes of drug delivery. The dosage forms of this invention include dosage forms in which the same drug is used in both the immediate-release and the prolonged-release portions as well as those in which one drug is formulated for immediate release and another drug, different from the first, is formulated for prolonged release. This invention encompasses dosage forms in which the immediate-release drug is at most sparingly soluble in water, i.e., either sparingly soluble or insoluble in water, while the prolonged-release drug can be of any level of solubility.

C. Controlled Release Formulations and Drug Delivery Systems

The formulations of the present invention can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that preferably, although not necessarily, results in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material which provides sustained release properties to the compounds. As such, the compounds for use the method of the invention can be administered in the form of microparticles for example, by injection or in the form of wafers or discs by implantation.

The term delayed release is used herein in its conventional sense to refer to a drug formulation that provides for an initial release of the drug after some delay following drug administration and that preferably, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

As compared with traditional drug delivery approaches, some controlled release technologies rely upon the modification of both macromolecules and synthetic small molecules to allow them to be actively instead of passively absorbed into the body. For example, XenoPort Inc. utilizes technology that takes existing molecules and re-engineers them to create new chemical entities (unique molecules) that have improved pharmacologic properties to either: 1) lengthen the short half-life of a drug; 2) overcome poor absorption; and/or 3) deal with poor drug distribution to target tissues. Techniques to lengthen the short half-life of a drug include the use of prodrugs with slow cleavage rates to release drugs over time or that engage transporters in small and large intestines to allow the use of oral sustained delivery systems, as well as drugs that engage active transport systems. Examples of such controlled release formulations, tablets, dosage forms, and drug delivery systems, and that are suitable for use with the present invention, are described in the following published US and PCT patent applications assigned to Xenoport Inc.: US20030158254; US20030158089; US20030017964; US2003130246; WO02100172; WO02100392; WO02100347; WO02100344; WO0242414; WO0228881; WO0228882; WO0244324; WO0232376; WO0228883; and WO0228411. In particular, Xenoport's XP13512 is a transported Prodrug of gabapentin that has been engineered to utilize high capacity transport mechanisms located in both the small and large intestine and to rapidly convert to gabapentin once in the body. In contrast to gabapentin itself, XP13512 was shown in preclinical and clinical studies to produce dose proportional blood levels of gabapentin across a broad range of oral doses, and to be absorbed efficiently from the large intestine.

Some other controlled release technologies rely upon methods that promote or enhance gastric retention, such as those developed by Depomed Inc. Because many drugs are best absorbed in the stomach and upper portions of the small intestine, Depomed has developed tablets that swell in the stomach during the postprandial or fed mode so that they are treated like undigested food. These tablets therefore sit safely and neutrally in the stomach for 6, 8, or more hours and deliver drug at a desired rate and time to upper gastrointestinal sites. Specific technologies in this area include: 1) tablets that slowly erode in gastric fluids to deliver drugs at almost a constant rate (particularly useful for highly insoluble drugs); 2) bi-layer tablets that combine drugs with different characteristics into a single table (such as a highly insoluble drug in an erosion layer and a soluble drug in a diffusion layer for sustained release of both); and 3) combination tablets that can either deliver drugs simultaneously or in sequence over a desired period of time (including an initial burst of a fast acting drug followed by slow and sustained delivery of another drug). Examples of such controlled release formulations that are suitable for use with the present invention and that rely upon gastric retention during the postprandial or fed mode, include tablets, dosage forms, and drug delivery systems in the following US patents assigned to Depomed Inc.: U.S. Pat. No. 6,488,962; U.S. Pat. No. 6,451,808; U.S. Pat. No. 6,340,475; U.S. Pat. No. 5,972,389; U.S. Pat. No. 5,582,837; and U.S. Pat. No. 5,007,790. Examples of such controlled release formulations that are suitable for use with the present invention and that rely upon gastric retention during the postprandial or fed mode, include tablets, dosage forms, and drug delivery systems in the following published US and PCT patent applications assigned to Depomed Inc.: US20030147952; US20030104062; US20030104053; US20030104052; US20030091630; US20030044466; US20030039688; US20020051820; WO0335040; WO0335039; WO0156544; WO0132217; WO9855107; WO9747285; and WO9318755.

Other controlled release systems include those developed by ALZA Corporation based upon: 1) osmotic technology for oral delivery; 2) transdermal delivery via patches; 3) liposomal delivery via intravenous injection; 4) osmotic technology for long-term delivery via implants; and 5) depot technology designed to deliver agents for periods of days to a month. ALZA oral delivery systems include those that employ osmosis to provide precise, controlled drug delivery for up to 24 hours for both poorly soluble and highly soluble drugs, as well as those that deliver high drug doses meeting high drug loading requirements. ALZA controlled transdermal delivery systems provide drug delivery through intact skin for as long as one week with a single application to improve drug absorption and deliver constant amounts of drug into the bloodstream over time. ALZA liposomal delivery systems involve lipid nanoparticles that evade recognition by the immune system because of their unique polyethylene glycol (PEG) coating, allowing the precise delivery of drugs to disease-specific areas of the body. ALZA also has developed osmotically driven systems to enable the continuous delivery of small drugs, peptides, proteins, DNA and other bioactive macromolecules for up to one year for systemic or tissue-specific therapy. Finally, ALZA depot injection therapy is designed to deliver biopharmaceutical agents and small molecules for periods of days to a month using a nonaqueous polymer solution for the stabilization of macromolecules and a unique delivery profile.

Examples of controlled release formulations, tablets, dosage forms, and drug delivery systems that are suitable for use with the present invention are described in the following US patents assigned to ALZA Corporation: U.S. Pat. No. 4,367,741; U.S. Pat. No. 4,402,695; U.S. Pat. No. 4,418,038; U.S. Pat. No. 4,434,153; U.S. Pat. No. 4,439,199; U.S. Pat. No. 4,450,198; U.S. Pat. No. 4,455,142; U.S. Pat. No. 4,455,144; U.S. Pat. No. 4,484,923; U.S. Pat. No. 4,486,193; U.S. Pat. No. 4,489,197; U.S. Pat. No. 4,511,353; U.S. Pat. No. 4,519,801; U.S. Pat. No. 4,526,578; U.S. Pat. No. 4,526,933; U.S. Pat. No. 4,534,757; U.S. Pat. No. 4,553,973; U.S. Pat. No. 4,559,222; U.S. Pat. No. 4,564,364; U.S. Pat. No. 4,578,075; U.S. Pat. No. 4,588,580; U.S. Pat. No. 4,610,686; U.S. Pat. No. 4,612,008; U.S. Pat. No. 4,618,487; U.S. Pat. No. 4,627,851; U.S. Pat. No. 4,629,449; U.S. Pat. No. 4,642,233; U.S. Pat. No. 4,649,043; U.S. Pat. No. 4,650,484; U.S. Pat. No. 4,659,558; U.S. Pat. No. 4,661,105; U.S. Pat. No. 4,662,880; U.S. Pat. No. 4,675,174; U.S. Pat. No. 4,681,583; U.S. Pat. No. 4,684,524; U.S. Pat. No. 4,692,336; U.S. Pat. No. 4,693,895; U.S. Pat. No. 4,704,119; U.S. Pat. No. 4,705,515; U.S. Pat. No. 4,717,566; U.S. Pat. No. 4,721,613; U.S. Pat. No. 4,723,957; U.S. Pat. No. 4,725,272; U.S. Pat. No. 4,728,498; U.S. Pat. No. 4,743,248; U.S. Pat. No. 4,747,847; U.S. Pat. No. 4,751,071; U.S. Pat. No. 4,753,802; U.S. Pat. No. 4,755,180; U.S. Pat. No. 4,756,314; U.S. Pat. No. 4,764,380; U.S. Pat. No. 4,773,907; U.S. Pat. No. 4,777,049; U.S. Pat. No. 4,781,924; U.S. Pat. No. 4,783,337; U.S. Pat. No. 4,786,503; U.S. Pat. No. 4,788,062; U.S. Pat. No. 4,810,502; U.S. Pat. No. 4,812,313; U.S. Pat. No. 4,816,258; U.S. Pat. No. 4,824,675; U.S. Pat. No. 4,834,979; U.S. Pat. No. 4,837,027; U.S. Pat. No. 4,842,867; U.S. Pat. No. 4,846,826; U.S. Pat. No. 4,847,093; U.S. Pat. No. 4,849,226; U.S. Pat. No. 4,851,229; U.S. Pat. No. 4,851,231; U.S. Pat. No. 4,851,232; U.S. Pat. No. 4,853,229; U.S. Pat. No. 4,857,330; U.S. Pat. No. 4,859,470; U.S. Pat. No. 4,863,456; U.S. Pat. No. 4,863,744; U.S. Pat. No. 4,865,598; U.S. Pat. No. 4,867,969; U.S. Pat. No. 4,871,548; U.S. Pat. No. 4,872,873; U.S. Pat. No. 4,874,388; U.S. Pat. No. 4,876,093; U.S. Pat. No. 4,892,778; U.S. Pat. No. 4,902,514; U.S. Pat. No. 4,904,474; U.S. Pat. No. 4,913,903; U.S. Pat. No. 4,915,949; U.S. Pat. No. 4,915,952; U.S. Pat. No. 4,917,895; U.S. Pat. No. 4,931,285; U.S. Pat. No. 4,946,685; U.S. Pat. No. 4,948,592; U.S. Pat. No. 4,954,344; U.S. Pat. No. 4,957,494; U.S. Pat. No. 4,960,416; U.S. Pat. No. 4,961,931; U.S. Pat. No. 4,961,932; U.S. Pat. No. 4,963,141; U.S. Pat. No. 4,966,769; U.S. Pat. No. 4,971,790; U.S. Pat. No. 4,976,966; U.S. Pat. No. 4,986,987; U.S. Pat. No. 5,006,346; U.S. Pat. No. 5,017,381; U.S. Pat. No. 5,019,397; U.S. Pat. No. 5,023,076; U.S. Pat. No. 5,023,088; U.S. Pat. No. 5,024,842; U.S. Pat. No. 5,028,434; U.S. Pat. No. 5,030,454; U.S. Pat. No. 5,071,656; U.S. Pat. No. 5,077,054; U.S. Pat. No. 5,082,668; U.S. Pat. No. 5,104,390; U.S. Pat. No. 5,110,597; U.S. Pat. No. 5,122,128; U.S. Pat. No. 5,125,894; U.S. Pat. No. 5,141,750; U.S. Pat. No. 5,141,752; U.S. Pat. No. 5,156,850; U.S. Pat. No. 5,160,743; U.S. Pat. No. 5,160,744; U.S. Pat. No. 5,169,382; U.S. Pat. No. 5,171,576; U.S. Pat. No. 5,176,665; U.S. Pat. No. 5,185,158; U.S. Pat. No. 5,190,765; U.S. Pat. No. 5,198,223; U.S. Pat. No. 5,198,229; U.S. Pat. No. 5,200,195; U.S. Pat. No. 5,200,196; U.S. Pat. No. 5,204,116; U.S. Pat. No. 5,208,037; U.S. Pat. No. 5,209,746; U.S. Pat. No. 5,221,254; U.S. Pat. No. 5,221,278; U.S. Pat. No. 5,229,133; U.S. Pat. No. 5,232,438; U.S. Pat. No. 5,232,705; U.S. Pat. No. 5,236,689; U.S. Pat. No. 5,236,714; U.S. Pat. No. 5,240,713; U.S. Pat. No. 5,246,710; U.S. Pat. No. 5,246,711; U.S. Pat. No. 5,252,338; U.S. Pat. No. 5,254,349; U.S. Pat. No. 5,266,332; U.S. Pat. No. 5,273,752; U.S. Pat. No. 5,284,660; U.S. Pat. No. 5,286,491; U.S. Pat. No. 5,308,348; U.S. Pat. No. 5,318,558; U.S. Pat. No. 5,320,850; U.S. Pat. No. 5,322,502; U.S. Pat. No. 5,326,571; U.S. Pat. No. 5,330,762; U.S. Pat. No. 5,338,550; U.S. Pat. No. 5,340,590; U.S. Pat. No. 5,342,623; U.S. Pat. No. 5,344,656; U.S. Pat. No. 5,348,746; U.S. Pat. No. 5,358,721; U.S. Pat. No. 5,364,630; U.S. Pat. No. 5,376,377; U.S. Pat. No. 5,391,381; U.S. Pat. No. 5,402,777; U.S. Pat. No. 5,403,275; U.S. Pat. No. 5,411,740; U.S. Pat. No. 5,417,675; U.S. Pat. No. 5,417,676; U.S. Pat. No. 5,417,682; U.S. Pat. No. 5,423,739; U.S. Pat. No. 5,424,289; U.S. Pat. No. 5,431,919; U.S. Pat. No. 5,443,442; U.S. Pat. No. 5,443,459; U.S. Pat. No. 5,443,461; U.S. Pat. No. 5,456,679; U.S. Pat. No. 5,460,826; U.S. Pat. No. 5,462,741; U.S. Pat. No. 5,462,745; U.S. Pat. No. 5,489,281; U.S. Pat. No. 5,499,979; U.S. Pat. No. 5,500,222; U.S. Pat. No. 5,512,293; U.S. Pat. No. 5,512,299; U.S. Pat. No. 5,529,787; U.S. Pat. No. 5,531,736; U.S. Pat. No. 5,532,003; U.S. Pat. No. 5,533,971; U.S. Pat. No. 5,534,263; U.S. Pat. No. 5,540,912; U.S. Pat. No. 5,543,156; U.S. Pat. No. 5,571,525; U.S. Pat. No. 5,573,503; U.S. Pat. No. 5,591,124; U.S. Pat. No. 5,593,695; U.S. Pat. No. 5,595,759; U.S. Pat. No. 5,603,954; U.S. Pat. No. 5,607,696; U.S. Pat. No. 5,609,885; U.S. Pat. No. 5,614,211; U.S. Pat. No. 5,614,578; U.S. Pat. No. 5,620,705; U.S. Pat. No. 5,620,708; U.S. Pat. No. 5,622,530; U.S. Pat. No. 5,622,944; U.S. Pat. No. 5,633,011; U.S. Pat. No. 5,639,477; U.S. Pat. No. 5,660,861; U.S. Pat. No. 5,667,804; U.S. Pat. No. 5,667,805; U.S. Pat. No. 5,674,895; U.S. Pat. No. 5,688,518; U.S. Pat. No. 5,698,224; U.S. Pat. No. 5,702,725; U.S. Pat. No. 5,702,727; U.S. Pat. No. 5,707,663; U.S. Pat. No. 5,713,852; U.S. Pat. No. 5,718,700; U.S. Pat. No. 5,736,580; U.S. Pat. No. 5,770,227; U.S. Pat. No. 5,780,058; U.S. Pat. No. 5,783,213; U.S. Pat. No. 5,785,994; U.S. Pat. No. 5,795,591; U.S. Pat. No. 5,811,465; U.S. Pat. No. 5,817,624; U.S. Pat. No. 5,824,340; U.S. Pat. No. 5,830,501; U.S. Pat. No. 5,830,502; U.S. Pat. No. 5,840,754; U.S. Pat. No. 5,858,407; U.S. Pat. No. 5,861,439; U.S. Pat. No. 5,863,558; U.S. Pat. No. 5,876,750; U.S. Pat. No. 5,883,135; U.S. Pat. No. 5,840,754; U.S. Pat. No. 5,897,878; U.S. Pat. No. 5,904,934; U.S. Pat. No. 5,904,935; U.S. Pat. No. 5,906,832; U.S. Pat. No. 5,912,268; U.S. Pat. No. 5,914,131; U.S. Pat. No. 5,916,582; U.S. Pat. No. 5,932,547; U.S. Pat. No. 5,938,654; U.S. Pat. No. 5,941,844; U.S. Pat. No. 5,955,103; U.S. Pat. No. 5,972,369; U.S. Pat. No. 5,972,370; U.S. Pat. No. 5,972,379; U.S. Pat. No. 5,980,943; U.S. Pat. No. 5,981,489; U.S. Pat. No. 5,983,130; U.S. Pat. No. 5,989,590; U.S. Pat. No. 5,995,869; U.S. Pat. No. 5,997,902; U.S. Pat. No. 6,001,390; U.S. Pat. No. 6,004,309; U.S. Pat. No. 6,004,578; U.S. Pat. No. 6,008,187; U.S. Pat. No. 6,020,000; U.S. Pat. No. 6,034,101; U.S. Pat. No. 6,036,973; U.S. Pat. No. 6,039,977; U.S. Pat. No. 6,057,374; U.S. Pat. No. 6,066,619; U.S. Pat. No. 6,068,850; U.S. Pat. No. 6,077,538; U.S. Pat. No. 6,083,190; U.S. Pat. No. 6,096,339; U.S. Pat. No. 6,106,845; U.S. Pat. No. 6,110,499; U.S. Pat. No. 6,120,798; U.S. Pat. No. 6,120,803; U.S. Pat. No. 6,124,261; U.S. Pat. No. 6,124,355; U.S. Pat. No. 6,130,200; U.S. Pat. No. 6,146,662; U.S. Pat. No. 6,153,678; U.S. Pat. No. 6,174,547; U.S. Pat. No. 6,183,466; U.S. Pat. No. 6,203,817; U.S. Pat. No. 6,210,712; U.S. Pat. No. 6,210,713; U.S. Pat. No. 6,224,907; U.S. Pat. No. 6,235,712; U.S. Pat. No. 6,245,357; U.S. Pat. No. 6,262,115; U.S. Pat. No. 6,264,990; U.S. Pat. No. 6,267,984; U.S. Pat. No. 6,287,598; U.S. Pat. No. 6,289,241; U.S. Pat. No. 6,331,311; U.S. Pat. No. 6,333,050; U.S. Pat. No. 6,342,249; U.S. Pat. No. 6,346,270; U.S. Pat. No. 6,365,183; U.S. Pat. No. 6,368,626; U.S. Pat. No. 6,387,403; U.S. Pat. No. 6,419,952; U.S. Pat. No. 6,440,457; U.S. Pat. No. 6,468,961; U.S. Pat. No. 6,491,683; U.S. Pat. No. 6,512,010; U.S. Pat. No. 6,514,530; U.S. Pat. No. 6,534,089; U.S. Pat. No. 6,544,252; U.S. Pat. No. 6,548,083; U.S. Pat. No. 6,551,613; U.S. Pat. No. 6,572,879; and U.S. Pat. No. 6,596,314.

Other examples of controlled release formulations, tablets, dosage forms, and drug delivery systems that are suitable for use with the present invention are described in the following published US patent application and PCT applications assigned to ALZA Corporation: US20010051183; WO0004886; WO0013663; WO0013674; WO0025753; WO0025790; WO0035419; WO0038650; WO0040218; WO0045790; WO0066126; WO0074650; WO019337; WO0119352; WO0121211; WO0137815; WO0141742; WO0143721; WO0156543; WO3041684; WO03041685; WO03041757; WO03045352; WO03051341; WO03053400; WO03053401; WO9000416; WO9004965; WO9113613; WO9116884; WO9204011; WO9211843; WO9212692; WO9213521; WO9217239; WO9218102; WO9300071; WO9305843; WO9306819; WO9314813; WO9319739; WO9320127; WO9320134; WO9407562; WO9408572; WO9416699; WO9421262; WO9427587; WO9427589; WO9503823; WO9519174; WO9529665; WO9600065; WO9613248; WO9625922; WO9637202; WO9640049; WO9640050; WO9640139; WO9640364; WO9640365; WO9703634; WO9800158; WO9802169; WO9814168; WO9816250; WO9817315; WO9827962; WO9827963; WO9843611; WO9907342; WO9912526; WO9912527; WO9918159; WO9929297; WO9929348; WO9932096; WO9932153; WO9948494; WO9956730; WO9958115; and WO9962496.

Another drug delivery technology suitable for use in the present invention is that disclosed by DepoMed, Inc. in U.S. Pat. No. 6,682,759, which discloses a method for manufacturing a pharmaceutical tablet for oral administration combining both immediate-release and prolonged-release modes of drug delivery. The tablet according to the method comprises a prolonged-release drug core and an immediate-release drug coating or layer, which can be insoluble or sparingly soluble in water. The method limits the drug particle diameter in the immediate-release coating or layer to 10 microns or less. The coating or layer is either the particles themselves, applied as an aqueous suspension, or a solid composition that contains the drug particles incorporated in a solid material that disintegrates rapidly in gastric fluid.

Andrx Corporation has also developed drug delivery technology suitable for use in the present invention that includes: 1) a pelletized pulsatile delivery system (“PPDS”); 2) a single composition osmotic tablet system (“SCOT”); 3) a solubility modulating hydrogel system (“SMHS”); 4) a delayed pulsatile hydrogel system (“DPHS”); 5) a stabilized pellet delivery system (“SPDS”); 6) a granulated modulating hydrogel system (“GMHS”); 7) a pelletized tablet system (“PELTAB”); 8) a porous tablet system (“PORTAB”); and 9) a stabilized tablet delivery system (“STDS”). PPDS uses pellets that are coated with specific polymers and agents to control the release rate of the microencapsulated drug and is designed for use with drugs that require a pulsed release. SCOT utilizes various osmotic modulating agents as well as polymer coatings to provide a zero-order drug release. SMHS utilizes a hydrogel-based dosage system that avoids the “initial burst effect” commonly observed with other sustained-release hydrogel formulations and that provides for sustained release without the need to use special coatings or structures that add to the cost of manufacturing. DPHS is designed for use with hydrogel matrix products characterized by an initial zero-order drug release followed by a rapid release that is achieved by the blending of selected hydrogel polymers to achieve a delayed pulse. SPDS incorporates a pellet core of drug and protective polymer outer layer, and is designed specifically for unstable drugs, while GMHS incorporates hydrogel and binding polymers with the drug and forms granules that are pressed into tablet form. PELTAB provides controlled release by using a water insoluble polymer to coat discrete drug crystals or pellets to enable them to resist the action of fluids in the gastrointestinal tract, and these coated pellets are then compressed into tablets. PORTAB provides controlled release by incorporating an osmotic core with a continuous polymer coating and a water soluble component that expands the core and creates microporous channels through which drug is released. Finally, STDS includes a dual layer coating technique that avoids the need to use a coating layer to separate the enteric coating layer from the omeprazole core.

Examples of controlled release formulations, tablets, dosage forms, and drug delivery systems that are suitable for use with the present invention are described in the following US patents assigned to Andrx Corporation: U.S. Pat. No. 5,397,574; U.S. Pat. No. 5,419,917; U.S. Pat. No. 5,458,887; U.S. Pat. No. 5,458,888; U.S. Pat. No. 5,472,708; U.S. Pat. No. 5,508,040; U.S. Pat. No. 5,558,879; U.S. Pat. No. 5,567,441; U.S. Pat. No. 5,654,005; U.S. Pat. No. 5,728,402; U.S. Pat. No. 5,736,159; U.S. Pat. No. 5,830,503; U.S. Pat. No. 5,834,023; U.S. Pat. No. 5,837,379; U.S. Pat. No. 5,916,595; U.S. Pat. No. 5,922,352; U.S. Pat. No. 6,099,859; U.S. Pat. No. 6,099,862; U.S. Pat. No. 6,103,263; U.S. Pat. No. 6,106,862; U.S. Pat. No. 6,156,342; U.S. Pat. No. 6,177,102; U.S. Pat. No. 6,197,347; U.S. Pat. No. 6,210,716; U.S. Pat. No. 6,238,703; U.S. Pat. No. 6,270,805; U.S. Pat. No. 6,284,275; U.S. Pat. No. 6,485,748; U.S. Pat. No. 6,495,162; U.S. Pat. No. 6,524,620; U.S. Pat. No. 6,544,556; U.S. Pat. No. 6,589,553; U.S. Pat. No. 6,602,522; and U.S. Pat. No. 6,610,326.

Examples of controlled release formulations, tablets, dosage forms, and drug delivery systems that are suitable for use with the present invention are described in the following published US and PCT patent applications assigned to Andrx Corporation: US20010024659; US20020115718; US20020156066; WO0004883; WO0009091; WO0012097; WO0027370; WO0050010; WO0132161; WO0134123; WO0236077; WO0236100; WO02062299; WO02062824; WO02065991; WO02069888; WO02074285; WO03000177; WO9521607; WO9629992; WO9633700; WO9640080; WO9748386; WO9833488; WO9833489; WO9930692; WO9947125; and WO9961005.

Some other examples of drug delivery approaches focus on non-oral drug delivery, providing parenteral, transmucosal, and topical delivery of proteins, peptides, and small molecules. For example, the Atrigel™ drug delivery system marketed by Atrix Laboratories Inc. comprises biodegradable polymers, similar to those used in biodegradable sutures, dissolved in biocompatible carriers. These pharmaceuticals may be blended into a liquid delivery system at the time of manufacturing or, depending upon the product, may be added later by a physician at the time of use. Injection of the liquid product subcutaneously or intramuscularly through a small gauge needle, or placement into accessible tissue sites through a cannula, causes displacement of the carrier with water in the tissue fluids, and a subsequent precipitate to form from the polymer into a solid film or implant. The drug encapsulated within the implant is then released in a controlled manner as the polymer matrix biodegrades over a period ranging from days to months. Examples of such drug delivery systems include Atrix's Eligard™, Atridox™/Doxirobe™, Atrisorb™ FreeFlow™/Atrisorb™-D FreeFlow, bone growth products, and others as described in the following published US and PCT patent applications assigned to Atrix Laboratories Inc.: U.S. RE37950; U.S. Pat. No. 6,630,155; U.S. Pat. No. 6,566,144; U.S. Pat. No. 6,610,252; U.S. Pat. No. 6,565,874; U.S. Pat. No. 6,528,080; U.S. Pat. No. 6,461,631; U.S. Pat. No. 6,395,293; U.S. Pat. No. 6,261,583; U.S. Pat. No. 6,143,314; U.S. Pat. No. 6,120,789; U.S. Pat. No. 6,071,530; U.S. Pat. No. 5,990,194; U.S. Pat. No. 5,945,115; U.S. Pat. No. 5,888,533; U.S. Pat. No. 5,792,469; U.S. Pat. No. 5,780,044; U.S. Pat. No. 5,759,563; U.S. Pat. No. 5,744,153; U.S. Pat. No. 5,739,176; U.S. Pat. No. 5,736,152; U.S. Pat. No. 5,733,950; U.S. Pat. No. 5,702,716; U.S. Pat. No. 5,681,873; U.S. Pat. No. 5,660,849; U.S. Pat. No. 5,599,552; U.S. Pat. No. 5,487,897; U.S. Pat. No. 5,368,859; U.S. Pat. No. 5,340,849; U.S. Pat. No. 5,324,519; U.S. Pat. No. 5,278,202; U.S. Pat. No. 5,278,201; US20020114737, US20030195489; US20030133964; US20010042317; US20020090398; US20020001608; and US2001042317.

Atrix Laboratories Inc. also markets technology for the non-oral transmucosal delivery of drugs over a time period from minutes to hours. For example, Atrix's BEMA™ (Bioerodible Muco-Adhesive Disc) drug delivery system comprises pre-formed bioerodible discs for local or systemic delivery. Examples of such drug delivery systems include those as described in U.S. Pat. No. 6,245,345.

Other drug delivery systems marketed by Atrix Laboratories Inc. focus on topical drug delivery. For example, SMP™ (Solvent Particle System) allows the topical delivery of highly water-insoluble drugs. This product allows for a controlled amount of a dissolved drug to permeate the epidermal layer of the skin by combining the dissolved drug with a microparticle suspension of the drug. The SMP™ system works in stages whereby: 1) the product is applied to the skin surface; 2) the product near follicles concentrates at the skin pore; 3) the drug readily partitions into skin oils; and 4) the drug diffuses throughout the area. By contrast, MCA™ (Mucocutaneous Absorption System) is a water-resistant topical gel providing sustained drug delivery. MCA™ forms a tenacious film for either wet or dry surfaces where: 1) the product is applied to the skin or mucosal surface; 2) the product forms a tenacious moisture-resistant film; and 3) the adhered film provides sustained release of drug for a period from hours to days. Yet another product, BCP™ (Biocompatible Polymer System) provides a non-cytotoxic gel or liquid that is applied as a protective film for wound healing. Examples of these systems include Orajel™-Ultra Mouth Sore Medicine as well as those as described in the following published US patents and applications assigned to Atrix Laboratories Inc.: U.S. Pat. No. 6,537,565; U.S. Pat. No. 6,432,415; U.S. Pat. No. 6,355,657; U.S. Pat. No. 5,962,006; U.S. Pat. No. 5,725,491; U.S. Pat. No. 5,722,950; U.S. Pat. No. 5,717,030; U.S. Pat. No. 5,707,647; U.S. Pat. No. 5,632,727; and US20010033853.

Additional formulations and compositions available from Teva Pharmaceutical Industries Ltd., Warner Lambert & Co., and Godecke Aktiengesellshaft that include gabapentin and are useful in the present invention include those as described in the following US patents and published US and PCT patent applications: U.S. Pat. No. 6,531,509; U.S. Pat. No. 6,255,526; U.S. Pat. No. 6,054,482; US2003055109; US2002045662; US2002009115; WO 01/97782; WO 01/97612; EP 2001946364; WO 99/59573; and WO 99/59572.

Additional formulations and compositions that include oxybutynin and are useful in the present invention include those as described in the following US patents and published US and PCT patent applications: U.S. Pat. No. 5,834,010; U.S. Pat. No. 5,601,839; and U.S. Pat. No. 5,164,190.

More particularly, in a further embodiment, the prolonged-release portion of the dosage form can be a dosage form that delivers its drug to the digestive system continuously over a period of time of at least an hour and preferably several hours and the drug is formulated as described in U.S. patent application Ser. No. 20030104053. In said embodiment, the immediate-release portion of the dosage form can be a coating applied or deposited over the entire surface of a unitary prolonged-release core, or can be a single layer of a tablet constructed in two or more layers, one of the other layers of which is the prolonged-released portion and is formulated as described in U.S. patent application Ser. No. 20030104053.

In another embodiment of the invention, the supporting matrix in controlled-release tablets or controlled release portions of tablets is a material that swells upon contact with gastric fluid to a size that is large enough to promote retention in the stomach while the subject is in the digestive state, which is also referred to as the postprandial or “fed” mode. This is one of two modes of activity of the stomach that differ by their distinctive patterns of gastroduodenal motor activity. The “fed” mode is induced by food ingestion and begins with a rapid and profound change in the motor pattern of the upper gastrointestinal (GI) tract. The change consists of a reduction in the amplitude of the contractions that the stomach undergoes and a reduction in the pyloric opening to a partially closed state. The result is a sieving process that allows liquids and small particles to pass through the partially open pylorus while indigestible particles that are larger than the pylorus are retropelled and retained in the stomach. This process causes the stomach to retain particles that are greater than about 1 cm in size for about 4 to 6 hours. The controlled-release matrix in these embodiments of the invention is therefore selected as one that swells to a size large enough to be retropelled and thereby retained in the stomach, causing the prolonged release of the drug to occur in the stomach rather than in the intestines. Disclosures of oral dosage forms that swell to sizes that will prolong the residence time in the stomach are found in U.S. Pat. No. 6,448,962, U.S. Pat. No. 6,340,475, U.S. Pat. No. 5,007,790, U.S. Pat. No. 5,582,837, U.S. Pat. No. 5,972,389, PCT Patent Application WO 98/55107, U.S. patent application Ser. No. 20010018707, U.S. patent application Ser. No. 20020051820, U.S. patent application Ser. No. 20030029688, U.S. patent application Ser. No. 20030044466, U.S. patent application Ser. No. 20030104062, U.S. patent application Ser. No. 20030147952, U.S. patent application Ser. No. 20030104053, and PCT Patent Application WO 96/26718. In particular, gastric retained dosage formulations for specific drugs have also been described, for example, a gastric retained dosage formulation for gabapentin is disclosed in PCT Patent Application WO 03/035040.

D. Dosing

The therapeutically effective amount or dose of a compound of the present invention, including the peripherally restricted compounds and additional agents described herein, will depend on the age, sex and weight of the patient, the current medical condition of the patient and the nature of the 5-HT₃ mediated disorder being treated. The skilled artisan will be able to determine appropriate dosages depending on these and other factors.

In a particular embodiment, drug administration or dosing is on an as-needed basis, and does not involve chronic drug administration. With an immediate release dosage form, as-needed administration can involve drug administration immediately prior to commencement of an activity wherein suppression of the symptoms of overactive bladder would be desirable, but will generally be in the range of from about 0 minutes to about 10 hours prior to such an activity, preferably in the range of from about 0 minutes to about 5 hours prior to such an activity, most preferably in the range of from about 0 minutes to about 3 hours prior to such an activity.

As used herein, the language “as-needed dosing,” also known as “pro re nata,” “pm dosing,” and “on demand dosing or administration” includes the administration of a therapeutically effective dose of the compound(s) at some time prior to commencement of an activity wherein suppression of a 5-HT₃ mediated disorder, e.g., lower urinary tract disorder would be desirable. Administration can be immediately prior to such an activity, including about 0 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, or about 10 hours prior to such an activity, depending on the formulation.

As used herein, the language “continuous dosing” refers to the chronic administration of a selected active agent.

A suitable dose of the 5-HT₃ receptor antagonist can be in the range of from about 0.001 mg to about 500 mg per day, such as from about 0.01 mg to about 100 mg, for example, from about 0.05 mg to about 50 mg, such as about 0.5 mg to about 25 mg per day. The dose can be administered in a single dosage or in multiple dosages, for example from 1 to 4 or more times per day. When multiple dosages are used, the amount of each dosage can be the same or different.

A suitable dose of the additional agent, e.g., a NARI compound, can be in the range of from about 0.001 mg to about 1000 mg per day, such as from about 0.05 mg to about 500 mg, for example, from about 0.03 mg to about 300 mg, such as about 0.02 mg to about 200 mg per day. The dose can be administered in a single dosage or in multiple dosages, for example from 1 to 4 or more times per day. When multiple dosages are used, the amount of each dosage can be the same or different.

A suitable dose of the compound having both 5-HT₃ receptor antagonist and NARI activity can be in the range of from about 0.001 mg to about 1000 mg per day, such as from about 0.05 mg to about 500 mg, for example, from about 0.03 mg to about 300 mg, such as from about 0.02 mg to about 200 mg per day. In a particular embodiment, a suitable dose of the compound having both 5-HT₃ receptor antagonist and NARI activity can be in the range of from about 0.1 mg to about 50 mg per day, such as from about 0.5 mg to about 10 mg per, day such as about 0.5, 1, 2, 3, 4, 5, 6, 7, 8, 9, or 10 mg per day. The dose per day can be administered in a single dosage or in multiple dosages, for example from 1 to 4 or more times per day. When multiple dosages are used, the amount of each dosage can be the same or different. For example a dose of 1 mg per day can be administered as two 0.5 mg doses, with about a 12 hour interval between doses.

When the method of treatment comprises coadministration of an additional agent and a peripherally restricted 5-HT₃ receptor antagonist each dose can typically contain from about 0.001 mg to about 1000 mg, such as from about 0.05 mg to about 500 mg, for example, from about 0.03 mg to about 300 mg, such as about 0.02 mg to about to about 200 mg of the NARI and typically can contain from about 0.001 mg to about 500 mg, such as from about 0.01 mg to about 100 mg, for example, from about 0.05 mg to about 50 mg, such as about 0.5 mg to about 25 mg of the peripherally restricted 5-HT₃ receptor antagonist.

It is understood that the amount of compound dosed per day can be administered every day, every other day, every 2 days, every 3 days, every 4 days, every 5 days, etc. For example, with every other day administration, a 5 mg per day dose can be initiated on Monday with a first subsequent 5 mg per day dose administered on Wednesday, a second subsequent 5 mg per day dose administered on Friday, etc.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

E. Kits of the Invention

In another embodiment, the invention is directed to a packaged pharmaceutical composition for treating one or more 5-HT₃ mediated disorders in a subject, comprising a container holding a therapeutically effective amount of a peripherally-restricted 5-HT₃ receptor antagonist; and instructions for using the antagonist for treating one or more 5-HT₃ mediated disorders in a subject.

Another embodiment of the invention pertains to a packaged pharmaceutical composition for treating one or more 5-HT₃ mediated disorders in a subject, comprising a container holding a therapeutically effective amount of a peripherally-restricted 5-HT₃ receptor antagonist; and instructions for using the antagonist and an additional agent for treating one or more 5-HT₃ mediated disorders thereof in a subject.

In another embodiment, the invention is directed to a packaged pharmaceutical composition for treating a functional bowel disorder, e.g., IBS, e.g., IBS-d, in a subject, comprising a container holding a therapeutically effective amount of a peripherally-restricted 5-HT₃ receptor antagonist; and instructions for using the antagonist for treating a functional bowel disorder in a subject.

Another embodiment of the invention pertains to a packaged pharmaceutical composition for treating a functional bowel disorder, e.g., IBS, e.g., IBS-d, in a subject, comprising a container holding a therapeutically effective amount of a peripherally-restricted 5-HT₃ receptor antagonist; and instructions for using the antagonist and an additional agent for treating a functional bowel disorder in a subject.

In another embodiment, the invention is directed to a packaged pharmaceutical composition for treating at least one symptom of a lower urinary tract disorder in a subject, comprising a container holding a therapeutically effective amount of a peripherally-restricted 5-HT₃ receptor antagonist; and instructions for using the antagonist for treating at least one symptom of a lower urinary tract disorder in a subject.

Another embodiment of the invention pertains to a packaged pharmaceutical composition for treating at least one symptom of a lower urinary tract disorder in a subject, comprising a container holding a therapeutically effective amount of a peripherally-restricted 5-HT₃ receptor antagonist; and instructions for using the antagonist and an additional agent for treating at least one symptom of a lower urinary tract disorder in a subject.

In another embodiment, the invention is directed to a packaged pharmaceutical composition for treating urinary incontinence in a subject, comprising a container holding a therapeutically effective amount of a peripherally-restricted 5-HT₃ receptor antagonist; and instructions for using the antagonist for treating urinary incontinence in a subject.

Another embodiment of the invention pertains to a packaged pharmaceutical composition for treating urinary incontinence in a subject, comprising a container holding a therapeutically effective amount of a peripherally-restricted 5-HT₃ receptor antagonist; and instructions for using the antagonist and an additional agent for treating urinary incontinence in a subject.

In another embodiment, the invention is directed to a packaged pharmaceutical composition for treating nausea, vomiting, e.g., CFV, retching or any combination thereof in a subject, comprising a container holding a therapeutically effective amount of a peripherally-restricted 5-HT₃ receptor antagonist; and instructions for using the antagonist for treating nausea, vomiting, retching or any combination thereof in a subject.

Another embodiment of the invention pertains to a packaged pharmaceutical composition for treating nausea, vomiting, e.g., CFV, retching or any combination thereof in a subject, comprising a container holding a therapeutically effective amount of a peripherally-restricted 5-HT₃ receptor antagonist; and instructions for using the antagonist and an additional agent for treating nausea, vomiting, retching or any combination thereof in a subject.

Compounds can be in separate dosage forms or combined in a single dosage form. In other embodiments of the kits, the instructional insert further includes instructions for administration with an additional therapeutic agent as described herein.

It is understood that in practicing the method or using a kit of the present invention that administration encompasses administration by different individuals (e.g., the subject, physicians or other medical professionals) administering the same or different compounds.

The invention also relates to a method of processing a claim under a health insurance policy submitted by a claimant seeking reimbursement for costs associated with the treatment of one or more 5-HT₃ mediated disorders, as described herein.

In one embodiment, the method of processing a claim under a health insurance policy submitted by a claimant seeking reimbursement for costs associated with treatment of one or more 5-HT₃ mediated disorders, wherein said treatment comprises coadministering to a subject a first amount of a 5-HT₃ receptor antagonist and a second amount of a noradrenaline reuptake inhibitor, wherein the first and second amounts together comprise a therapeutically effective amount comprising: reviewing said claim; determining whether said treatment is reimbursable under said insurance policy; and processing said claim to provide partial or complete reimbursement of said costs.

The invention is further illustrated by the following examples, which should not be construed as further limiting the subject invention.

### EXAMPLES

### Example 1

### Synthetic Preparation of an MCI-225-OUAT

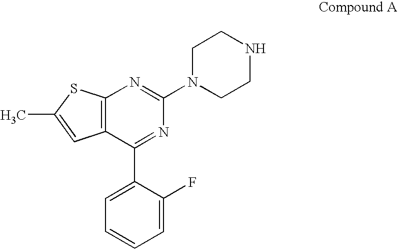

Compound A, 1.0 g, was combined with K₂CO₃, water (25 mL), and EtOAc (25 mL). The mixture was shaken in separatory funnel until all solids were dissolved. The organic phase was then removed. The aqueous phase was combined with an additional volume of EtOAc (25 mL) in the separatory funnel and shaken again. The organic phase was subsequently removed and combined with the first organic phase extraction.

The combined organics from both extractions were dried over MgSO4 with stirring. The combined organics were then filtered through CGF and concentrated to a solid, weighing 191 mg. The sold was then combined with K₂CO₃ (189 mg), MeI (86 uL) in DMF (4 mL). The reaction mixture is stirred at room temperature for 4 hrs, and monitored by TLC. The reaction mixture is then diluted with water (10 mL) and extracted with EtOAc (2×25 mL). The combined organics of this extraction were washed with water (50 mL), dried over MgSO4, and concentrated to an oily residue which solidified on standing at room temperature (183 mg)

This solid was then combined with MeI (330 uL), and acetone (4 mL), stirred at room temperature for 12 hr, and concentrated to a solid. The solid was then recrystallized in EtOAc/MeOH after filtration, forming a light yellow solid weighing 212 mg

Examples 2-6 teach assays for determining various properties of compounds of the invention. Preferable test compounds are those according to Formulae I-III, as described herein, in particular, the peripherally-restricted 5-HT3 antagonists described herein (e.g., MCI 225-QUAT).

### Example 2

### Assessment of Bioavailability of Test Compounds

Assessment of bioavailability from plasma concentration-time data involves determining the area under the plasma concentration-time curve (AUC). The AUC is directly proportional to the total amount of unchanged drug that reaches the systemic circulation. For an accurate measurement, blood is sampled frequently over a long enough time to observe virtually complete drug elimination.

Bioavailability is assessed after single and/or repetitive (multiple) dosing. More information about rate of absorption is available after a single dose than after multiple dosing. However, multiple dosing more closely represents the usual clinical situation, and plasma concentrations are usually higher than those after a single dose, facilitating data analysis. After multiple dosing at a fixed-dosing interval for four or five elimination half-lives, the blood drug concentration should be at steady state (the amount absorbed equals the amount eliminated within each dosing interval). The extent of absorption is then analyzed by measuring the AUC during a dosing interval. Measuring the AUC over 24 h is preferable because of circadian variations in physiologic functions and because of possible variations in dosing intervals and absorption rates during a day.

For drugs excreted primarily unchanged in urine, bioavailability is estimated by measuring the total amount of drug excreted after a single dose. Urine is collected over a period of, for example, 7 to 10 elimination half-lives for complete urinary recovery of the absorbed drug. Alternatively, bioavailability is assessed after multiple dosing by measuring unchanged drug recovered from urine over 24 h under steady-state conditions.

The area under the plot of plasma concentration of drug against time after drug administration. The ratio of the AUC after oral administration of a drug formulation to that after the intravenous injection of the same dose to the same subject is used during drug development to assess a drug's oral bioavailability.

To assess the bioavailability of a test compound, subjects (for example, 15-20 age and weight match subjects) are selected. Blood samples are drawn after fasting for >/=12 hours and then at specified time periods after ingesting an oral formulation of the test compound. HPLC is used to determine concentration of test compound in serum isolated from the blood samples. A similar test group of subject are administered an equivalent dosage of test compound formulated for intervenous delivery. Areas under the curve (AUC) are calculated to assess bioavailability. Bioavailability is determined as the ratio of the amount of compound absorbed from the oral formulation to the amount absorbed after intravenous administration.

Bioavalability can also be studied in appropriate animal models as follows. Animals (e.g., rats) are administered test compound orally (e.g., at dosages of 1, 3, 10 and 30 mg/kg) and the ED50 for a pharmacodynamic endpoint calculated. The ED50 is likewise determined for the response following i.v. administration of the compound (at the same or slightly higher doses). An exemplary pharmacodynamic endpoint for compounds having 5-HT3 receptor antagonist activity is the Bezold-Jarisch reflex described above. An ED50 following oral administration of, for example, 5 mg/kg can be compared to an ED50 following i.v. administration of, for example, 20 mg/kg. The data indicate that 25% of the test compound is becoming bioavailable, e.g., systemically available. Thus, oral administration would require 4× the dose needed i.v.

### Example 3

### Blood Brain Barrier Penetrance of a Test Compound

The blood brain barrier penetrance of a test compound can be determined using an analysis based on the analysis described in Drug Metab Dispos. 2000 February;28(2):205-8 by Le Doze F et al.

Glacial acetic acid, acetonitrile and ammonium acetate (Merck, Darmstadt, Germany), ascorbic acid (Fluka Chimie A G, Bucks, Switzerland), dimethyl sulfoxide (DMSO), and trisodium edetate (Prolabo, Fontenay sous Bois, France) are all of analytical grade. The HPLC system used is an isocratic pump (model L6000; Merck, Darmstadt, Germany) coupled to a photodiode array detector (model 996; Waters, Saint-Quentin en Yvelines, France) monitored by Millenium software (Waters).

Experiments are performed on 95 male Sprague-Dawley rats (CERJ, Le Genest Saint Isle, France) weighing 200 to 300 g. The rats are housed in groups of five and maintained under standard laboratory conditions (22±1° C., 12-h light/dark cycle, food and water ad libitum) before study.

Rats are give an i.p. injection (10 mg/kg b.wt.) of solution of the compound under investigation (2 mg/mL), in DMSO and are sacrificed by inhalation of carbon dioxide at 1, 2, 3, 5, 8, 12, 18, 24, 48, and 72 h after injection (five animals at each time).

Blood samples (2-3 mL) are taken by cardiac puncture and the blood vessels are rinsed with 0.9% saline. The brains are removed and kept at 4° C., and WM samples from the corpus callosum and GM samples from the frontal cortex are dissected out. The brain samples are weighed [weight (mean±S.D.) of WM samples], homogenized in 500 μl of an aqueous solution containing 0.5 mg/ml each of trisodium edetate and ascorbic acid using light-protected tubes. Blood samples are then centrifuged at 4000 rpm, and the tubes are stored at 20° C.

The concentrations of the compound under investigation are measured by HPLC (Wyss, 1990). Spiked standards and deproteinated rat samples are prepared in foil-lined tubes, working in a darkened room. Serum and tissue homogenate (200 μl) are mixed with 200 μl of acetonitrile, shaken, and centrifuged (4000 rpm for 10 min). An aliquot (50 μl) of the upper phase is then injected directly into the HPLC system, where the chromatographic conditions are: column, LC ABZ [15 cm×4.6 mm, i.d.; particle size, 5 μm; reversed phase C₁₈; (Supelco, St. Quentin Fallavier, France)]; the mobile phase is selected based on the particular compound analyzed; UV detection wavelength, 354 nM].

Standard curves are prepared by adding appropriate amounts (60 ng to 1.8 μg) of the compound under investigation in DMSO to serum blank samples (200 μl); and 30 to 900 ng of the compound under investigation to tissue homogenate blank samples (200 μl). An amount of internal standard is selected for the serum standard curves and for the tissue homogenate standard curves. Standard curves are run every day of determination. Two quality controls (low and high) are tested to estimate the reproducibility, precision, and reliability of the method.

The serum concentrations of drug are expressed in micrograms of drug per milliliter of serum. The tissue concentrations of drug are expressed in micrograms of drug per gram of wet tissue weight. Results are expressed as means±S.D. The mean coefficient of variation from five measures at each time gives the interindividual variance. Terminal half-lives (t_{1/2}) of the compound under investigation in serum and brain tissues are estimated by least-squares regression analysis of the terminal phase of the concentration-time curves. The area under the serum or brain concentration versus time curves (AUC)₀ values are determined by the trapezoid rule during the period of experiment and, when necessary, the infinite part of the curve is calculated as the estimated terminal serum concentration divided by the slope ke (ke=0.693/t_{1/2}). The mean concentrations on the WM and GM are compared using ANOVA with a balanced nested design. Data obtained at a given time are compared using a Mann-Whitney rank sum test. A value of P>0.02 is considered to be statistically insignificant.

As an alternative to serum concentration, the brain concentration can be compared to an aggregate of serum concentration and peripheral tissue concentration. Serum concentrations are determined, for example, as described above and select preipheral tissues are processes according to art recognized methodologies to determine tissue concentration of test compound.

### Example

### Receptor Binding Profiles of MCI 225 OUAT

The binding of MCI 225 QUAT to various receptors/transporters was compared to that of the non-quaternized parent compound, MCI-225. Binding assays were performed essentially according to procedures as follows.

The 5-HT₃ human recombinant binding assay was performed essentially as described in Lummis et al. (1990) Eur. J. Pharmacol. 189:223-27; Hoyer et al. (1988) Mol. Pharmacol. 33:303; and Tyers et al. (1991) Therapie 46:431-435. Briefly, human recombinant receptors were from HEK-293 cells. Tritiated radioligand (GR 65630) was used. % specific binding was determined as a function of log [compound]. MDL 72222 (1 alpha H, 3 alpha, 5 alpha H-tropan-3-yl-3,5-dichlorobenzoate) was used as nonspecific determinant, reference compound and positive control.

The norepinephrine transporter (NET) assay was performed essentially as described in Raisman et al. (1982) Eur. J. Pharmacol. 78:345-351 (minor modifications); and Raisman and Briley (1981) Eur. J. Pharmacol. 72:423. Briefly, human recombinant receptors were from CHO cells. Tritiated radioligand (nisoxetine) was used. % specific binding was determined as a function of log [compound]. Desipramine (DMI) was used as nonspecific determinant, reference compound and positive control.

The serotonin transporter (SERT) assay was performed essentially as described in D'Armato et al. (1987) J. Pharmacol. & Exp. Ther. 242:364-371 (with modification); and Brown et al. (1986) Eur. J. Pharmacol. 123:161-165. Briefly, human recombinant receptors were from human platelet membranes. Tritiated radioligand (N-methyl citalopram) was used. % specific binding was determined as a function of log [compound]. Imipramine was used as nonspecific determinant, reference compound and positive control.

The data are presented in Table I. The binding profile of MCI 225 QUAT differed from that of the parent compound with increased binding to 5-HT3 receptors and diminished binding to norepinephrine transporters.

TABLE I

| | Conc. | | | | | |
|---|---|---|---|---|---|---|
| | 1.00E−10 | 1.00E−10 | 1.00E−08 | 1.00E−08 | 1.00E−06 | 1.00E−06 |
| Cmpd. | 225 | 225q | 225 | 225q | 225 | 225q |
| 5-HT₃ (hr) | 0% | 15% | 22% | 33% | 100% | 101% |
| NET (hr) | 17% | −13% | 72% | −10% | 104% | 42% |
| SERT (h) | 1% | 7% | 10% | 11% | 94% | 32% |

### Example 5

### Treatment of Functional Bowel Disease

Rodent Model of Visceromotor Response to Colorectal Distension (CRD)

The ability of a test compound to reverse acetic acid-induced colonic hypersensitivity in a rodent model of irritable bowel syndrome is assessed. Specifically, the experiments described herein investigate the effect of a test compound on visceromotor responses in a rat model of acetic acid-induced colonic hypersensitivity in the distal colon of non-stressed rats.

Adult male Fisher rats are housed (2 per cage) in the animal facility at standard conditions. Following one week of acclimatization to the animal facility, the rats are brought to the laboratory and are handled daily for another week to get used to the environment and the research associate performing the experiments.

The visceromotor behavioral response to colorectal distension is measured by counting the number of abdominal contractions recorded by a strain gauge sutured onto the abdominal musculature as described in Gunter et al., Physiol. Behav., 69(3): 379-82 (2000) in awake unrestrained animals. A 5 cm latex balloon catheter inserted via the anal canal into the colon is used for colorectal distensions. Constant pressure tonic distensions are performed in a graded manner (15, 30 or 60 mmHg) and are maintained for a period of 10 min and the numbers of abdominal muscle contractions are recorded to measure the level of colonic sensation. A 10 min recovery is allowed between distensions.

Acetic acid-induced colonic hypersensitivity in rats has been described by Langlois et al., Eur. J. Pharmacol., 318: 141-144 (1996) and Plourde et al., Am. J. Physiol. 273: G191-G196 (1997). In this study, a low concentration of acetic acid (1.5 ml, 0.6%) is administered intracolonically to sensitize the colon without causing histological damage to the colonic mucosa as described in previous studies (Gunter et al., supra).

A test compound (30 mg/kg; n=6) or vehicle alone (n=4) is administered to the rats intraperitoneally (i.p.) 30 min prior to initiation of the protocol for colorectal distension. Injection volume is 0.2 mL using 100% propylene glycol as the vehicle. Three consecutive colorectal distensions at 15, 30 or 60 mmHg applied at 10-min intervals are recorded. Visceromotor responses are evaluated as the number of abdominal muscle contractions recorded during the 10-min periods of colorectal distension. Non-sensitized and sensitized uninjected control animals served to demonstrate the lower and upper levels of response, respectively (n=2/group).

Acetic acid is known to reliably sensitize rat visceromotor responses to CRD. Vehicle alone has no effect on the response to CRD in acetic acid sensitized animals. Effective compounds eliminate the visceromotor response to CRD in, e.g., 50% of the animals tested. The model is predictive of drug efficacy in treating IBS in humans.

In other examples, the ability of a test compound to effect increased colonic tansit is assessed. The model used provides a method of determining the ability of the test compound to normalize accelerated colonic transit induced by water avoidance stress (WAS). The test compound is assayed alone or in comparison with known 5-HT3 receptor antagonists. The model provides a method of evaluating the effectiveness of the compound in a specific patient group of IBS sufferers where stress induced colonic motility is considered a significant contributing factor.

Preliminary testing in the water avoidance stress model confirms that there exists an association between stress and altered colonic motility. Fecal pellet output is measured by counting the total number of fecal pellets produced during 1 hour of WAS. Adult male F-344 rats, supplied by Charles River Laboratories and weighing 270-350 g, are used in this study. The rats are housed 2 per cage under standard conditions. Following one to two weeks of acclimatization to the animal facility, the rats are brought to the laboratory and handled daily for another week to acclimatize them to laboratory conditions and to the research associate who performs the studies. All procedures used in this study are approved in accordance with facility standards.

To acclimatize the animals prior to experimental testing, all rats undergo sham stress (1-hour in stress chamber without water) for 2-4 consecutive days before undergoing WAS (sham is performed until rats produce 0-1 pellet per hour for 2 consecutive days). At the end of the 1-hour stress period, the fecal pellets are counted and recorded.

WAS causes an acceleration of colonic transit, which can be quantified by counting the number of fecal pellets, produced during the stress procedure. Rats are placed for 1-hour into a stress chamber onto a raised platform 7.5 cm×.7.5 cm×9 cm (L×W×H) in the center of a stress chamber filled with room temperature water 8 cm in depth. The stress chamber is constructed from a rectangular plastic tub (40.2×60.2×31.2 cm). (100% propylene glycol serves as a vehicle control). Compounds are tested at, for example, doses of 1, 3, 10 and 30 mg/kg. All drugs and the vehicle are administered as an i.p. injection.

In properly controlled experiments, there is no significant difference in the number of fecal pellets produced in 1 hour between the animals in their home cage or the sham stress control group. Upon exposure to a WAS (WAS basal) for 1 hour, there is a highly significant (p<0.001) increase in fecal pellet output compared to fecal pellet output from rats in their home cage or the sham stress control group. After acclimation to the stress chamber for 2-4 days the fecal pellet output of the WAS vehicle treatment group is not statistically different from the fecal pellet output of the non-treated WAS group

In rats pretreated with test compound (or known 5-HT3 receptor antagonist and then placed on the WAS, the number of fecal pellets produced during 1 hour is significantly less than the number produced during WAS in the vehicle treated group. Statistical significance is assessed using one-way ANOVA followed by Tukey post-test. Statistical differences are compared between the WAS groups and the sham stress group and are considered significant if p<0.05, e.g., p<0.05, p<0.01, p<0.001.

These experiments demonstrate that stress, in this case a water avoidance stressor, causes a significant increase in colonic transit as demonstrated by an increase in fecal pellet output. Compounds that significantly inhibit the stress-induced increase in fecal pellet production (e.g., to an extent that resembles that observed with known antagonists) can be used as a suitable therapy for the treatment of non-constipated IBS.

### Example 6

### Treatment of Overactive Bladder

The acute models described below provide methods for evaluating compounds of the invention for the treatment of overactive bladder. Briefly, the models provide a method for reducing the bladder capacity of test animals by infusing either protamine sulfate and potassium chloride (See, Chuang, Y. C. et al., Urology 61(3): 664-670 (2003)) or dilute acetic acid (See, Sasaki, K. et al., J. Urol. 168(3): 1259-1264 (2002)) into the bladder. The infusates cause irritation of the bladder and a reduction in bladder capacity by selectively activating bladder afferent fibers, such as C-fiber afferents. Following irritation of the bladder, a test compound can be administered and the ability of the compound to reverse (partially or totally) the reduction in bladder capacity resulting from the irritation, can be determined. Compounds which reverse the reduction in bladder capacity can be used in the treatment of overactive bladder.

The chronic model of neurogenic bladder described below, in which C-fiber afferents are chronically activated as a result of spinal cord injury provide further methods for evaluating compounds of the invention for the treatment of overactive bladder. (See, Yoshiyama, M. et al., Urology 54(5): 929-933 (1999)). Following spinal cord injury, a test compound can be administered and the ability of the compound to reverse (partially or totally) the reduction in bladder capacity resulting from spinal cord injury can be determined. Compounds which reverse the reduction in bladder capacity can be used in the treatment of overactive bladder, for example, neurogenic bladder.

A. Dilute Acetic Acid Model-Rats

Female rats (250-275 g BW, n=8) are anesthetized with urethane (1.2 g/kg) and a saline-filled catheter (PE-50) is inserted into the proximal duodenum for intraduodenal drug administration. A flared-tipped PE-50 catheter is inserted into the bladder dome, via a midline lower abdominal incision, for bladder filling and pressure recording and is secured by ligation. The abdominal cavity is moistened with saline and is closed by covering with a thin plastic sheet in order to maintain access to the bladder for emptying purposes. Fine silver or stainless steel wire electrodes are inserted into the external urethral sphincter (EUS) percutaneously for electromyography (EMG). Animals are positioned on a heating pad which is maintained body temperature at 37° C.

Saline and all subsequent infusates are continuously infused at a rate of about 0.055 ml/min via the bladder filling catheter for about 60 minutes to obtain a baseline of lower urinary tract activity (continuous cystometry; CMG). At the end of the control saline cystometry period, the infusion pump is stopped, the bladder is emptied by fluid withdrawal via the infusion catheter, and a single filling cystometrogram is performed using saline at the same flow rate as the continuous infusion, in order to measure bladder capacity. Bladder capacity (ml) is calculated as the flow rate of the bladder filling solution (ml/min) multiplied by the elapsed time between commencement of bladder filling and occurrence of bladder contraction (min).

Following the control period, a 0.25% acetic acid solution in saline (AA) is infused into the bladder to induce bladder irritation. Following 30 minutes of AA infusion, 3 vehicle injections (10% TWEEN™ 80 in saline, 1 ml/kg dose) are administered intraduodenally at 20 minute intervals to determine vehicle effects on the intercontraction interval and to achieve a stable level of irritation with the dilute acetic acid solution. Following injection of the third vehicle control, bladder capacity is again measured, as described above but using AA to fill the bladder. Increasing doses of the compound of the invention (e.g., 3, 10 or 30 mg/kg, as a 1 ml/kg dose) are then administered intraduodenally at 60 minute intervals in order to construct a cumulative dose-response relationship. Bladder capacity is measured as described above using AA to fill the bladder, at 20 and 50 minutes following each subsequent drug treatment.

Bladder capacity is determined for each treatment regimen as described above (flow rate of the bladder filling solution (ml/min) multiplied by the elapsed time between commencement of bladder filling and occurrence of bladder contraction (min)) and is converted to % Bladder Capacity normalized to the last vehicle measurement of the AA/Veh 3 treatment group. Data are then analyzed by non-parametric ANOVA for repeated measures (Friedman Test) with Dunn's Multiple Comparison test. All comparisons are made from the last vehicle measurement (AA/Veh 3), e.g., P,0.050=significant).

Compounds resulting in a dose-dependent increase in bladder capacity can be used in the treatment of lower urinary tract disorders in a subject. In particular, the effectiveness of compounds in this model is predictive of efficacy in the treatment of lower urinary tract disorders in humans.

B. Dilute Acetic Acid Model-Cats

The ability of a test compound to reverse the reduction in bladder capacity is tested in the following continuous infusion of dilute acetic acid in a cat model, a commonly used model of overactive bladder (Thor and Katofiasc, 1995, J. Pharmacol. Exptl. Ther. 274: 1014-24).

Six alpha-chloralose anesthetized (50-100 mg/kg) normal female cats (2.5-3.5 kg; Harlan) are utilized in this study. A test compound is dissolved in 5% methylcellulose in water (or other suitable diluent) at increasing concentrations (e.g., 3.0, 10.0 or 30 mg/ml). Animals are dosed by volume of injection=body weight in kg.

The following morning after which female cats are to have their food removed, the cats are anesthetized with isoflurane and prepped for surgery using aseptic technique. Polyethylene catheters are surgically placed to permit the measurement of bladder pressure, urethral pressure, arterial pressure, respiratory rate as well as for the delivery of drugs. Fine wire electrodes are implanted alongside the external urethral anal sphincter. Following surgery, the cats are slowly switched from the gas anesthetic isoflurane (2-3.5%) to alpha-chloralose (50-100 mg/kg). During control cystometry, saline is slowly infused into the bladder (0.5-1.0 ml/min) for 1 hour. The control cystometry is followed by 0.5% acetic acid in saline for the duration of the experiment. After assessing the cystometric variables under these baseline conditions, the effects of the test compound on bladder capacity are determined (e.g., via a 3 point dose response protocol).

Data is analyzed using a non-parametric One-Way ANOVA (Friedman Test) with the post-hoc Dunn's multiple comparison t test. P<0.05 is considered significant.

The ability of the test compound to cause a significant dose-dependent increase in bladder capacity following acetic acid irritation is determined. The data that is obtained may be used to support findings in the rat, e.g., demonstrating that a compound is effective in increasing bladder capacity in commonly utilized models of OAB in two species. These results are also predictive of the efficacy of the comopund in the treatment of BPH, for example, the irritative symptoms of BPH.

C. Protamine Sulfate/Physiological Urinary Potassium Model

Female rats are prepared as described for the dilute acetic acid model (rats). Following the control period, a 10 mg/mL protamine sulfate (PS) in saline solution is infused for about 30 minutes in order to permeabilize the urothelial diffusion barrier. After PS treatment, the infusate is switched to 300 mM KCl in saline to induce bladder irritation. Once a stable level of lower urinary tract hyperactivity is established (20-30 minutes), vehicle injections are made and the effects of the vehicle are assessed as decribed for the acetic acid model. The cumulative dose-response relationship with test compound is determined as described. The reamining data are collected (e.g., cystometrogram) in order to determine changes in bladder capacity caused by the irritation protocol and subsequent drug administration. This model acutely activates bladder afferent fibers, including, C-fiber afferents. The effectiveness of compounds in this model is predictive of efficacy in the treatment of lower urinary tract disorders in humans.

D. Chronic Modelfor Overactive Bladder: Chronic Spinal Cord Injury Model

Female Sprague-Dawley rats (Charles River, 250-300 g) are anesthetized with isofluorane (4%) and a laminectomy is performed at the T9-10 spinal level. The spinal cord is transected and the intervening space filled with Gelfoam. The overlying muscle layers and skin are sequentially closed with suture, and the animals are treated with antibiotic (100 mg/kg ampicillin s.c.). Residual urine is expressed prior to returning the animals to their home cages, and thereafter 3 times daily until terminal experimentation four weeks later. On the day of the experiment, the animals are anesthetized with isofluorane (4%) and a jugular catheter (PE10) is inserted for access to the systemic circulation and tunneled subcutaneously to exit through the midscapular region. Via a midline abdominal incision, a PE50 catheter with a fire-flared tip is inserted into the dome of the bladder through a small cystotomy and secured by ligation for bladder filling and pressure recording. Small diameter (75 μm) stainless steel wires are inserted percutaneously into the external urethral sphincter (EUS) for electromyography (EMG). The abdominal wall and the overlying skin of the neck and abdomen are closed with suture and the animal is mounted in a Ballman-type restraint cage. A water bottle is positioned within easy reach of the animal's mouth for ad libitum access to water. The bladder catheter is hooked up to the perfusion pump and pressure transducer, and the EUS-EMG electrodes to their amplifier. Following a 30 minute recovery from anesthesia and acclimatization, normal saline is infused at a constant rate (0.100-0.150 ml/min) for control cystometric recording.

Following a 60-90 minute control period of normal saline infusion (0.100-0.150 ml/min) to collect baseline continuous open cystometric data, the pump is turned off, the bladder is emptied, the pump turned back on, and bladder capacity is estimated by a filling cystometrogram. At 3×20-30 minute intervals, vehicle is administered intravenously in order to ascertain vehicle effects on bladder activity. Following the third vehicle control, bladder capacity is again estimated as described above. Subsequently, a cumulative dose-response is performed with the agent of choice. Bladder capacity is measured 20 minutes following each dose. This is a model of neurogenic bladder, in which C-fiber afferents are chronically activated.

### Example 7

### Treatment of Vomiting and Retching

The suitability of a compound of the invention to reduce retching and vomiting therapeutically is assessed in an accepted animal model of cytotoxin-induced emesis. Specifically, the experiments described herein are used to investigate the effect of a compound to reduce cisplatin-induced retching and/or vomiting in the ferret. Ondansetron is used as a positive control in the model, in view of its known antiemetic activity (Rudd and Naylor, Eur. J. Pharmacol., 322: 79-82 (1997)).

Adult male ferrets (Mustela putario furo) weighing 1200-1880g are purchased from Triple F Farns (Sayre, Pa.) and housed in individual cages at standardized conditions (12:12 h light/dark cycle and 21-23° C.). Prior to the experiments, the ferrets are allowed a 7-10 day acclimatization period to the animal facility. The ferrets are fed a carnivore diet with free access to food and water throughout the course of the study. The use of the ferret model of emesis and the drug treatment are preapproved in accordance with facility standards.

Experiments are performed essentially as follows. A cisplatin solution is prepared by adding preheated (70° C.) saline to cisplatin powder (Sigma-Aldrich Co.) and stirring or sonicating at 40° C. until dissolved. Test compounds are likewise solubilized under suitable conditions. Following administration of the cisplatin and either the test compound, ondansetron (control) or vehicle alone, the occurrence of retching and vomiting is monitored for a period of 6 hours. Retching is defined as the number of forceful rhythmic contractions of the abdomen occurring with the animal in characteristic posture, but not resulting in the expulsion of upper gastrointestinal tract contents (Watson et al., British Journal of Pharmacology, 115(1): 84-94 (1994)). Vomiting is defined as the forceful oral expulsion of upper gastrointestinal contents. The latency of the retching or vomiting response and the number of episodes are recorded for each animal and are summarized for each experimental group (Wright et al., Infect. Immun., 68(4): 2386-9 (2000)).

In an exemplary experiment, ferrets are given one hour of acclimation to the observation cage. Following acclimation, ferrets are given an intraperitoneal (i.p.) injection of cisplatin (5 mg/kg in 5 mL) that is followed, after about 2 minutes, by i.p. injection of a single dose of test compound or ondansetron. Dose-response effects of the test compound dosed at 1, 10 and 30 mg/kg i.p. in a 0.5 mL/kg solution or ondansetron dosed at 5 and 10 mg/kg i.p. in a 0.5 mL/kg solution are studied. Each animal is given a single-dose drug treatment. In addition, three animals are given an initial dose (30 mg/kg i.p.) and a second injection (30 mg/kg i.p.) of test compound at 180 minutes following the initial dose. Control animals are treated with cisplatin followed by vehicle alone (propanediol dosed in a 0.5 mL/kg solution). All groups are randomized.

Cisplatin is expected to induce an emetic response in 100% of the animals receiving vehicle alone. The mean responseis characterized by the total number of events (both retches and vomits) which occur during the observation period. The mean latency of the first response post-cicplatin administration is also determined. Ondansetron that is applied at the 5 mg/kg and 10 mg/kg dose is expected to reduce the number of emetic events and increase the latency of the first emetic response induced by cisplatin.

The dose-dependent reduction in the retches and vomits induced by cisplatin is determined at concentrations of test compound of 1, 10 or 30 mg/kg. Compounds effective at reducing retching and vomiting using a similar dose range as the positive control can be used in the treatment of nausea, vomiting, retching or any combination thereof in a subject.

Equivalents

The skilled artisan will appreciate that the methodologies featured in the above examples are also suitable for testing and/or characterizing other peripherally restricted 5-HT₃ antagonists featured in the present invention and/or combinations of agents as described herein.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including reaction times, reaction size/volume, and experimental reagents, such as solvents, catalysts, pressures, atmospheric conditions, e.g., nitrogen atmosphere, and reducing/oxidizing agents, etc., with art-recognized alternatives and using no more than routine experimentation, are within the scope of the present application. It should also be understood that assays used to determine the effectiveness of 5-HT₃ peripherally restricted compounds on the 5-HT₃ mediated disorders described herein are intended to be predictive of efficacy in humans.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values that fall within these ranges, as well as the upper or lower limits of a range of values, are also contemplated by the present application.

### INCORPORATION BY REFERENCE

The contents of all references, issued patents, and published patent applications cited throughout this application are hereby expressly incorporated herein in their entireties by reference.

1. A method for treating, preventing or reducing one or more 5-HT₃ mediated disorders in a subject in need thereof comprising administering to said subject a therapeutically effective amount of a compound selected from the group consisting of:
(a) a quaternary ammonium derivative of MCI-225 (MCI-225-QUAT);
(b) a quaternary ammonium derivative of MCI-225 (MCI-225-QUAT) possessing an enhanced therapeutic profile;
(c) a peripherally-restricted 5-HT₃ receptor antagonist together with an additional agent for treating, preventing or reducing the one or more 5-HT₃ mediated disorders in the subject;
(d) a peripherally-restricted 5-HT₃ receptor antagonist possessing an enhanced therapeutic profile together with an additional agent for treating, preventing or reducing the one or more 5-HT₃ mediated disorders in the subject; and
(e) a peripherally-restricted 5-HT₃ receptor antagonist together with an additional agent for treating, preventing or reducing the one or more 5-HT₃ mediated disorders in the subject, wherein the peripherally-restricted 5-HT₃ receptor antagonist possesses an enhanced therapeutic profile resulting from the combination of the peripherally-restricted 5-HT₃ receptor antagonist with the additional agent.

2. The method of claim 1, wherein the 5-HT3 mediated disorder is selected from the group consisting of functional bowel disorder, symptoms of a lower urinary tract disorder, nausea, vomiting, retching, overactive bladder (OAB), stress urinary incontinence, pain, fibromyalgia and depressive conditions, obesity and weight gain, pre-menstrual syndrome, eating disorders, migraine, Parkinson's disease, stroke, schizophrenia, obsessive-compulsive disorder, fatigue, and any combination thereof.

3-18. (canceled)

19. A method for treating one or more 5-HT3 mediated disorders in a subject in need thereof comprising administering to said subject a therapeutically effective amount of a compound selected from the group consisting of
(a) a compound of Formula I: or a pharmaceutically acceptable salt thereof;
(b) a compound of Formula II: or a pharmaceutically acceptable salt thereof; and
(c) a compound of Formula III: or a pharmaceutically acceptable salt thereof.

20. The method of claim 19, wherein R₁ is a C₁-C₆ alkyl group and Ar is a substituted phenyl.

21. The method of claim 19, wherein the substituted phenyl group is substituted with a halogen.

22. The method of claim 19, wherein n is 2, R₁ is a C₁-C₆ alkyl group and Ar is phenyl substituted with fluorine.

23. The method of claim 19, wherein n is 2, the substituted phenyl group is substituted with a halogen and R₁ is a methyl group.

24. The method of claim 19, wherein R₂ is hydrogen.

25. The method of claim 19, wherein, if present, R¹ is CH₃, R₂ is H, Ar is 2-fluoro-phenylene, R³ and R⁴ are H, R⁵ and R⁶ are CH₃, n is 2, A⁻ is I⁻, Z is N⁺(C₁₋₄)₂, and Y is a quaternary ammonium salt, the salt of a carboxylic acid, the salt of a sulfonic acid, or the salt of a phosphoric acid.

26. The method of claim 19, wherein the 5-HT₃ mediated disorder is selected from the group consisting of functional bowel disorder, symptoms of a lower urinary tract disorder, nausea, vomiting, retching, overactive bladder (OAB), stress urinary incontinence, pain, fibromyalgia and depressive conditions, obesity and weight gain, pre-menstrual syndrome, eating disorders, migraine, Parkinson's disease, stroke, schizophrenia, obsessive-compulsive disorder, fatigue, and any combination thereof.

27-30. (canceled)

31. A packaged pharmaceutical composition for treating one or more 5-HT₃ mediated disorders in a subject, comprising a container holding a therapeutically effective amount of a peripherally-restricted 5-HT₃ receptor antagonist; and instructions for using the composition for treating the one or more 5-HT₃ mediated disorders in the subject.

32. The packaged pharmaceutical composition of claim 31, further comprising an additional agent for treating the one or more 5-HT₃ mediated disorders thereof in the subject.

33. The packaged pharmaceutical of claim 31, wherein the 5-HT₃ mediated disorder is selected from the group consisting of functional bowel disorder, symptoms of a lower urinary tract disorder, nausea, vomiting, retching, overactive bladder (OAB), stress urinary incontinence, pain, fibromyalgia and depressive conditions, obesity and weight gain, pre-menstrual syndrome, eating disorders, migraine, Parkinson's disease, stroke, schizophrenia, obsessive-compulsive disorder, fatigue, and any combination thereof.

34. A pharmaceutical composition for treating one or more 5-HT₃ mediated disorders in a subject, comprising a compound selected from the group consisting of:
(a) a peripherally-restricted 5-HT₃ receptor antagonist selected based on its peripheral restriction,
(b) an MCI-225-QUAT;
(c) an MCI-225-QUAT possessing an enhanced therapeutic profile; and
(d) a peripherally-restricted 5-HT₃ receptor antagonist together with an additional agent for treating the one or more 5-HT₃ mediated disorders in the subject;
and a pharmaceutically acceptable carrier.

35-37. (canceled)

38. The pharmaceutical composition of claim 34, wherein the 5-HT₃ mediated disorder is selected from the group consisting of functional bowel disorder, symptoms of a lower urinary tract disorder, nausea, vomiting, retching, overactive bladder (OAB), stress urinary incontinence, pain, fibromyalgia and depressive conditions, obesity and weight gain, pre-menstrual syndrome, eating disorders, migraine, Parkinson's disease, stroke, schizophrenia, obsessive-compulsive disorder, fatigue, and any combination thereof.

39. A method for treating a functional bowel disorder in a subject in need thereof comprising administering to said subject a therapeutically effective amount of a compound selected from the group consisting of:
(a) a quaternary ammonium derivative of MCI-225 (MCI-225-QUAT);
(b) a peripherally-restricted 5-HT₃ receptor antagonist together with an additional agent for treating the functional bowel disorder in the subject; and
(c) a peripherally-restricted 5-HT₃ receptor antagonist together with a noradrenaline reuptake inhibitor.

40. The method of claim 39, wherein the functional bowel disorder is selected from the group consisting of (a) irritable bowel syndrome, (b) diarrhea-predominant irritable bowel syndrome, (c) alternating constipation/diarrhea irritable bowel syndrome, and (d) nonconstipated irritable bowel syndrome.

41-59. (canceled)

60. A method for treating a functional bowel disorder in a subject in need thereof comprising administering to said subject a therapeutically effective amount of a compound selected from the group consisting of
(a) a compound of Formula I: or a pharmaceutically acceptable salt thereof;
(b) a compound of Formula II: or a pharmaceutically acceptable salt thereof; and
(c) a compound of Formula III: or a pharmaceutically acceptable salt thereof.

61. The method of claim 60, wherein R₁ is a C₁-C₆ alkyl group and Ar is a substituted phenyl.

62. The method of claim 60, wherein the substituted phenyl group is substituted with a halogen.

63. The method of claim 60, wherein n is 2, R₁ is a C₁-C₆ alkyl group and Ar is phenyl substituted with fluorine.

64. The method of claim 60, wherein n is 2, the substituted phenyl group is substituted with a halogen and R₁ is a methyl group.

65. The method of claim 60, wherein R₂ is hydrogen.

66. The method of claim 60, wherein, if present, R¹ is CH₃, R₂ is H, Ar is 2-fluoro-phenylene, R³ and R⁴ are H, R⁵ and R⁶ are CH₃, n is 2, A⁻ is I⁻, Z is N⁺(C₁₋₄)₂, and Y is a quaternary ammonium salt, the salt of a carboxylic acid, the salt of a sulfonic acid, or the salt of a phosphoric acid.

67. A method of treating at least one symptom of a lower urinary tract disorder in a subject in need thereof comprising administering to said subject a therapeutically effective amount of a quaternary ammonium derivative of MCI-225 (MCI-225-QUAT).

68-82. (canceled)

83. A method of treating at least one symptom of a lower urinary tract disorder in a subject in need thereof comprising administering to said subject a therapeutically effective amount of a compound selected from the group consisting of
(a) a compound of Formula I: or a pharmaceutically acceptable salt thereof,
(b) a compound of Formula II: or a pharmaceutically acceptable salt thereof, and
(c) a compound of Formula III: or a pharmaceutically acceptable salt thereof.

84. The method of claim 83, wherein R₁ is a C₁-C₆ alkyl group and Ar is a substituted phenyl.

85. The method of claim 83, wherein the substituted phenyl group is substituted with a halogen.

86. The method of claim 83, wherein n is 2, R₁ is a C₁-C₆ alkyl group and Ar is phenyl substituted with fluorine.

87. The method of claim 83, wherein n is 2, the substituted phenyl group is substituted with a halogen and R₁ is a methyl group.

88. The method of claim 83, wherein R₂ is hydrogen.

89. The method of claim 83, wherein, if present, R¹ is CH₃, R₂ is H, Ar is 2-fluoro-phenylene, R³ and R⁴ are H, R⁵ and R⁶ are CH₃, n is 2, A⁻ is I⁻, Z is N⁺(C₁₋₄)₂, and Y is a quaternary ammonium salt, the salt of a carboxylic acid, the salt of a sulfonic acid, or the salt of a phosphoric acid.

90. A method for treating urinary incontinence in a subject in need thereof comprising administering to said subject a therapeutically effective amount of a compound selected from the group consisting of:
(a) a quaternary ammonium derivative of MCI-225 (MCI-225-QUAT);
(b) a quaternary ammonium derivative of MCI-225 (MCI-225-QUAT) possessing an enhanced therapeutic profile;
(c) a peripherally-restricted 5-HT₃ receptor antagonist together with an additional agent for treating the urinary incontinence in the subject; and
(d) a peripherally-restricted 5-HT₃ receptor antagonist together with a noradrenaline reuptake inhibitor.

91. (canceled)

92. A method for treating nausea, vomiting, retching or any combination thereof in a subject in need thereof comprising administering to said subject a therapeutically effective amount of (a) a quaternary ammonium derivative of MCI-225 (MCI-225-QUAT); or (b) a quaternary ammonium derivative of MCI-225 (MCI-225-QUAT) possessing an enhanced therapeutic profile.

93. (canceled)

94. The method of claims 92, wherein the nausea, vomiting, retching or any combination thereof is caused by an anesthetic, radiation, a cancer chemotherapeutic agent, a toxic agent, an odor, a medicine, pregnancy or motion.

95. The method of claim 92, wherein the medicine is selected from the group consisting of an analgesic, an antibiotic, an antifungal, a serotonin reuptake inhibitor, or a dual serotonin-norepinephrine reuptake inhibitor (SNRI).

96. The method of claim 94, wherein the nausea, vomiting, retching or any combination thereof is caused by (a) a condition which is associated with vertigo, (b) headache, or (c) a malady of the gastrointestinal (GI) tract.

97-99. (canceled)

100. The method of claim 92, wherein the nausea, vomiting, retching or any combination thereof is chronic functional vomiting.

101-115. (canceled)

116. A method for treating nausea, vomiting, retching or any combination thereof in a subject in need thereof comprising administering to said subject a t therapeutically effective amount of a compound selected from the group consisting of
(a) a compound of Formula I: or a pharmaceutically acceptable salt thereof;
(b) a compound of Formula II: or a pharmaceutically acceptable salt thereof; and
(c) a compound of Formula III: or a pharmaceutically acceptable salt thereof.

117. The method of claim 116, wherein R₁ is a C₁-C₆ alkyl group and Ar is a substituted phenyl.

118. The method of claim 116, wherein the substituted phenyl group is substituted with a halogen.

119. The method of claim 116, wherein n is 2, R₁ is a C₁-C₆ alkyl group and Ar is phenyl substituted with fluorine.

120. The method of claim 116, wherein n is 2, the substituted phenyl group is substituted with a halogen and R₁ is a methyl group.

121. The method of claim 116, wherein R₂ is hydrogen.

122. The method of claim 116, wherein, if present, R¹ is CH₃, R₂ is H, Ar is 2-fluoro-phenylene, R³ and R⁴ are H, R⁵ and R⁶ are CH₃, n is 2, A⁻ is I⁻, Z is N⁺(C₁₋₄)₂, and Y is a quaternary ammonium salt, the salt of a carboxylic acid, the salt of a sulfonic acid, or the salt of a phosphoric acid.

123-129. (canceled)

130. A method of treating at least one symptom of a lower urinary tract disorder in a subject in need of treatment, wherein the symptom is selected from the group consisting of urinary frequency, urinary urgency, nocturia and enuresis, comprising coadministering to said subject a peripherally-restricted 5-HT₃ receptor antagonist with an additional agent.

131. The method of claim 130, wherein the additional agent is a noradrenaline reuptake inhibitor.

132. The method of claim 130, wherein the lower urinary tract disorder is selected from the group consisting of overactive bladder, interstitial cystitis, prostatitis, prostadynia and benign prostatic hyperplasia.

133-137. (canceled)

138. The method of claim 90, wherein the urinary incontinence is stress urinary incontinence.

139. (canceled)

140. A method for treating nausea, vomiting, retching or any combination thereof in a subject in need thereof comprising administering to said subject a compound selected from the group consisting of:
(a) a peripherally-restricted 5-HT₃ receptor antagonist together with an additional agent for treating the nausea, vomiting, retching or any combination thereof in the subject;
(b) a peripherally-restricted 5-HT₃ receptor antagonist together with a noradrenaline reuptake inhibitor;
(c) a peripherally-restricted 5-HT₃ receptor antagonist selected based on its peripheral restriction; and
(d) a peripherally-restricted 5-HT₃ receptor antagonist together with an additional agent, wherein the additional agent is selected based on its effect in combination with the peripherally-restricted 5-HT₃ receptor antagonist for treating the nausea, vomiting, retching or any combination thereof in the subject.

141-143. (canceled)

144. The method of claims 140, wherein the nausea, vomiting, retching or any combination thereof is chronic functional vomiting.

145-159. (canceled)

160. A compound selected from the group consisting of
(a) Formula I: or a pharmaceutically acceptable salt thereof;
(b) Formula II: or a pharmaceutically acceptable salt thereof; and
(c) Formula III: or a pharmaceutically acceptable salt thereof.

161. The compound of claim 160, wherein R₁ is a C₁-C₆ alkyl group and Ar is a substituted phenyl.

162. The compound of claim 160, wherein the substituted phenyl group is substituted with a halogen.

163. The compound of claim 160, wherein n is 2, R₁ is a C₁-C₆ alkyl group and Ar is phenyl substituted with fluorine.

164. The compound of claim 160, wherein n is 2, the substituted phenyl group is substituted with a halogen and R₁ is a methyl group.

165. The compound of claim 160, wherein R₂ is hydrogen.

166. The compound of claim 160, wherein, if present, R₁ is CH₃, R₂ is H, Ar is 2-fluoro-phenylene, R³ and R⁴ are H, R⁵ and R⁶ are CH₃, n is 2, A⁻ is I⁻, Z is N⁺(C₁₋₄)₂, and Y is a quaternary ammonium salt, the salt of a carboxylic acid, the salt of a sulfonic acid, or the salt of a phosphoric acid.

167. A packaged pharmaceutical composition for treating a functional bowel disorder in a subject, comprising a container holding a therapeutically effective amount of a peripherally-restricted 5-HT₃ receptor antagonist; and instructions for using the composition for treating the functional bowel disorder in the subject.

168. The packaged pharmaceutical composition of claim 167 further comprising an additional agent for treating the functional bowel disorder in the subject.

169. A pharmaceutical composition for treating a functional bowel disorder in a subject, comprising a compound selected from the group consisting of:
(a) a peripherally-restricted 5-HT₃ receptor antagonist selected based on its peripheral restriction;
(b) an MCI-225-QUAT;
(c) an MCI-225-QUAT possessing an enhanced therapeutic profile; and
(d) a peripherally-restricted 5-HT₃ receptor antagonist together with an additional agent for treating the functional bowel disorder in the subject;
and a pharmaceutically acceptable carrier.

170-172. (canceled)

173. The pharmaceutical composition of claim 169, wherein the functional bowel disorder is selected from the group consisting of (a) irritable bowel syndrome, (b) diarrhea-predominant irritable bowel syndrome, (c) alternating constipation/diarrhea irritable bowel syndrome, and (d) nonconstipated irritable bowel syndrome.

174-176. (canceled)

177. (canceled)

178. A packaged pharmaceutical composition for treating at least one symptom of a lower urinary tract disorder in a subject, comprising a container holding a therapeutically effective amount of a peripherally-restricted 5-HT₃ receptor antagonist; and instructions for using the composition for treating the at least one symptom of a lower urinary tract disorder in the subject.

179. The packaged pharmaceutical composition of claim 178, further comprising an additional agent for treating the at least one symptom of a lower urinary tract disorder in the subject.

180. A pharmaceutical composition for treating at least one symptom of a lower urinary tract disorder in a subject in need of treatment, comprising a compound selected from the group consisting of:
(a) a peripherally-restricted 5-HT₃ receptor antagonist selected based on its peripheral restriction;
(b) an MCI-225-QUAT;
(c) an MCI-225-QUAT possessing an enhanced therapeutic profile; and
(d) a peripherally-restricted 5-HT₃ receptor antagonist together with an additional agent for treating the at least one symptom of a lower urinary tract disorder in the subject;
and a pharmacuetically acceptable carrier,
wherein the symptom is selected from the group consisting of urinary frequency, urinary urgency, nocturia and enuresis.

181-183. (canceled)

184. The pharmaceutical composition of claim 180, wherein the lower urinary tract disorder is selected from the group consisting of overactive bladder, interstitial cystitis, prostatitis, prostadynia and benign prostatic hyperplasia.

185-187. (canceled)

188. A packaged pharmaceutical composition for treating urinary incontinence in a subject, comprising a container holding a therapeutically effective amount of a peripherally-restricted 5-HT₃ receptor antagonist; and instructions for using the composition for treating urinary incontinence in a subject.

189. The packaged pharmaceutical composition of claim 188-together with an additional agent for treating urinary incontinence in a subject.

190. A pharmaceutical composition for treating urinary incontinence in a subject in need thereof, comprising a compound selected from the group consisting of:
(a) a peripherally-restricted 5-HT₃ receptor antagonist selected based on its peripheral restriction;
(b) an MCI-225-QUAT;
(c) an MCI-225-QUAT possessing an enhanced therapeutic profile;
(d) a peripherally-restricted 5-HT₃ receptor antagonist together with an additional agent for treating the urinary incontinence in the subject;
and a pharmaceutically acceptable carrier.

191-193. (canceled)

194. The pharmaceutical composition of claim 190, wherein the urinary incontinence is stress urinary incontinence.

195. (canceled)

196. A packaged pharmaceutical composition for treating nausea, vomiting, retching or any combination thereof in a subject, comprising a container holding a therapeutically effective amount of a peripherally-restricted 5-HT₃ receptor antagonist; and instructions for using the composition for treating the nausea, vomiting, retching or any combination thereof in the subject.

197. The packaged pharmaceutical composition of claim 196, together with an additional agent for treating the nausea, vomiting, retching or any combination thereof in the subject.

198. A pharmaceutical composition for treating nausea, vomiting, retching or any combination thereof in a subject, comprising a compound selected from the group consisting of:
(a) a peripherally-restricted 5-HT₃ receptor antagonist selected based on its peripheral restriction;
(b) an MCI-225-QUAT;
(c) an MCI-225-QUAT possessing an enhanced therapeutic profile;
(d) a peripherally-restricted 5-HT₃ receptor antagonist together with an additional agent for treating the nausea, vomiting, retching or any combination thereof in the subject;
(e) a peripherally-restricted 5-HT₃ receptor antagonist selected based on its peripheral restriction together with an additional agent for treating the nausea, vomiting, retching or any combination thereof in the subject; and
(f) a peripherally-restricted 5-HT₃ receptor antagonist selected based on its peripheral restriction together with an additional agent for treating the nausea, vomiting, retching or any combination thereof in the subject, wherein the additional agent is selected based on its effect in combination with the peripherally-restricted 5-HT₃ receptor antagonist;
and a pharmaceutically acceptable carrier.

199-203. (canceled)

204. The pharmaceutical composition of claim 198, wherein the nausea, vomiting, retching or any combination thereof is caused by an anesthetic, radiation, a cancer chemotherapeutic agent, a toxic agent, an odor, a medicine, pregnancy or motion.

205. The pharmaceutical composition of claim 204, wherein the medicine is selected from the group consisting of an analgesic, an antibiotic, an antifungal, a serotonin reuptake inhibitor, or a dual serotonin-norepinephrine reuptake inhibitor (SNRI).

206. The pharmaceutical composition of claim 198, wherein the nausea, vomiting, retching or any combination thereof is caused by (a) a condition which is associated with vertigo, (b) headache, or (c) a malady of the gastrointestinal (GI) tract.

207-208. (canceled)

209. The pharmaceutical composition of claim 198, wherein the nausea, vomiting, retching or any combination thereof is chronic functional vomiting.

210. (canceled)
